SEQUENCE LISTING (69 sequences)

SEQ ID NO: 1 - DNA, 3082 bp, synthetic construct
  misc_feature 1..3082: note = Description of Artificial Sequence: Synthetic polynucleotide
  source 1..3082: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagtggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 2 - DNA, 3290 bp, synthetic construct
  misc_feature 1..3290: note = Description of Artificial Sequence: Synthetic polynucleotide
  source 1..3290: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 3 - DNA, 3166 bp, synthetic construct
  misc_feature 1..3166: note = Description of Artificial Sequence: Synthetic polynucleotide
  source 1..3166: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 4 - DNA, 3374 bp, synthetic construct
  misc_feature 1..3374: note = Description of Artificial Sequence: Synthetic polynucleotide
  source 1..3374: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 5 - DNA, 3265 bp, synthetic construct
  misc_feature 1..3265: note = Description of Artificial Sequence: Synthetic polynucleotide
  source 1..3265: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 6 - DNA, 3473 bp, synthetic construct
  misc_feature 1..3473: note = Description of Artificial Sequence: Synthetic polynucleotide
  source 1..3473: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 7 - DNA, 3187 bp, synthetic construct
  misc_feature 1..3187: note = Description of Artificial Sequence: Synthetic polynucleotide
  source 1..3187: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 8 - DNA, 3395 bp, synthetic construct
  misc_feature 1..3395: note = Description of Artificial Sequence: Synthetic polynucleotide
  source 1..3395: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 9 - DNA, 3286 bp, synthetic construct
  misc_feature 1..3286: note = Description of Artificial Sequence: Synthetic polynucleotide
  source 1..3286: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 10 - DNA, 3494 bp, synthetic construct
  misc_feature 1..3494: note = Description of Artificial Sequence: Synthetic polynucleotide
  source 1..3494: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 11 - DNA, 2967 bp, synthetic construct
  misc_feature 1..2967: note = Description of Artificial Sequence: Synthetic polynucleotide
  source 1..2967: mol_type = other DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcgaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 12 - DNA, 3051 bp, synthetic construct
  misc_feature 1..3051: note = Description of Artificial Sequence: Synthetic polynucleotide
  source 1..3051: mol_type = other DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcgaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 13 - DNA, 3150 bp, synthetic construct
  misc_feature 1..3150: note = Description of Artificial Sequence: Synthetic polynucleotide
  source 1..3150: mol_type = other DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcgaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 14 - DNA, 3072 bp, synthetic construct
  misc_feature 1..3072: note = Description of Artificial Sequence: Synthetic polynucleotide
  source 1..3072: mol_type = other DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcgaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 15 - DNA, 3171 bp, synthetic construct
  misc_feature 1..3171: note = Description of Artificial Sequence: Synthetic polynucleotide
  source 1..3171: mol_type = other DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcgaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 16 - DNA, 2667 bp, synthetic construct
  misc_feature 1..2667: note = Description of Artificial Sequence: Synthetic polynucleotide
  source 1..2667: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagtggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 17 - DNA, 2875 bp, synthetic construct
  misc_feature 1..2875: note = Description of Artificial Sequence: Synthetic polynucleotide
  source 1..2875: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 18 - DNA, 2751 bp, synthetic construct
  misc_feature 1..2751: note = Description of Artificial Sequence: Synthetic polynucleotide
  source 1..2751: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 19 - DNA, 2959 bp, synthetic construct
  misc_feature 1..2959: note = Description of Artificial Sequence: Synthetic polynucleotide
  source 1..2959: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 20 - DNA, 2850 bp, synthetic construct
  misc_feature 1..2850: note = Description of Artificial Sequence: Synthetic polynucleotide
  source 1..2850: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 21 - DNA, 3058 bp, synthetic construct
  misc_feature 1..3058: note = Description of Artificial Sequence: Synthetic polynucleotide
  source 1..3058: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 22 - DNA, 2772 bp, synthetic construct
  misc_feature 1..2772: note = Description of Artificial Sequence: Synthetic polynucleotide
  source 1..2772: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 23 - DNA, 2980 bp, synthetic construct
  misc_feature 1..2980: note = Description of Artificial Sequence: Synthetic polynucleotide
  source 1..2980: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 24 - DNA, 2871 bp, synthetic construct
  misc_feature 1..2871: note = Description of Artificial Sequence: Synthetic polynucleotide
  source 1..2871: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 25 - DNA, 3079 bp, synthetic construct
  misc_feature 1..3079: note = Description of Artificial Sequence: Synthetic polynucleotide
  source 1..3079: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 26 - DNA, 2688 bp, synthetic construct
  misc_feature 1..2688: note = Description of Artificial Sequence: Synthetic polynucleotide
  source 1..2688: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagtggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 27 - DNA, 2896 bp, synthetic construct
  misc_feature 1..2896: note = Description of Artificial Sequence: Synthetic polynucleotide
  source 1..2896: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 28 - DNA, 2772 bp, synthetic construct
  misc_feature 1..2772: note = Description of Artificial Sequence: Synthetic polynucleotide
  source 1..2772: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 29 - DNA, 2980 bp, synthetic construct
  misc_feature 1..2980: note = Description of Artificial Sequence: Synthetic polynucleotide
  source 1..2980: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 30 - DNA, 2871 bp, synthetic construct
  misc_feature 1..2871: note = Description of Artificial Sequence: Synthetic polynucleotide
  source 1..2871: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 31 - DNA, 3079 bp, synthetic construct
  misc_feature 1..3079: note = Description of Artificial Sequence: Synthetic polynucleotide
  source 1..3079: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 32 - DNA, 2793 bp, synthetic construct
  misc_feature 1..2793: note = Description of Artificial Sequence: Synthetic polynucleotide
  source 1..2793: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 33 - DNA, 3001 bp, synthetic construct
  misc_feature 1..3001: note = Description of Artificial Sequence: Synthetic polynucleotide
  source 1..3001: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 34 - DNA, 2892 bp, synthetic construct
  misc_feature 1..2892: note = Description of Artificial Sequence: Synthetic polynucleotide
  source 1..2892: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 35 - DNA, 3100 bp, synthetic construct
  misc_feature 1..3100: note = Description of Artificial Sequence: Synthetic polynucleotide
  source 1..3100: mol_type = other DNA
gttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgtcgggctaagagaggcagcggcgagggcaggggaagtcttctaacatgcggggacgtggaggaaaatcccggccccatgtacgggaagattattttcgtgttgttgctcagtgagatcgtttctatctccgctgaagaccctcaaggcgacgccgctcaaaagacggacacgagccatcacgaccaagaccatcccacgtttaataaaataacacctaatctcgccgaatttgcattttcactgtataggcaactcgcccatcaaagcaattctacaaacattttctttagcccggtcagtatagcgactgctttcgccatgctgtctctcggtacaaaagccgatacccatgacgagattttggaaggactcaactttaatctgaccgaaatacccgaagcacaaattcacgaggggtttcaagagctgctgcgaactttgaatcaacccgattcccaactgcaactcacgacaggtaacgggttgtttctgagtgaagggcttaaactggttgacaaattcctggaagacgtgaagaaactctatcatagtgaggcatttacagttaattttggagataccgaggaagctaagaagcaaattaatgactatgttgagaaaggcacgcagggaaagatcgttgacctcgtgaaagaactggatcgagatacggtgttcgccctcgtcaactatatattcttcaaggggaagtgggaacgcccattcgaggtgaaagatacagaagaggaagattttcatgtagatcaagttacaacagtaaaagtacccatgatgaaaagactcgggatgttcaatatccaacattgcaagaagttgtcatcttgggtccttttgatgaagtatcttgggaacgcaacggctatattctttctcccggacgaaggcaagctgcaacatctcgagaacgagctgactcatgacattattacgaaatttcttgagaacgaggatcgccggagcgcgtccctgcacctgcctaagctcagcataacaggtacgtacgacctcaaatccgtgttgggacagttggggattacgaaagtgttttccaacggagcggatctgagcggtgtgaccgaagaagctccacttaaactgtcaaaagcggtccacaaagccgttttgactatagatgaaaagggtacagaggccgccggcgcgatgttcctggaagctatcccgatgtccataccaccagaagtgaaatttaataagcctttcgtgtttctgatgatagagcagaacacaaaatccccactgtttatgggcaaggtcgtcaacccaacacagaagggttcaggcgggtccggcggaagtgggccacttggtatgtggtctaggctgataatattcggcgttatggccggcgtgatcggtacaattctgctcatcagttatgggatacggagattgatcaagaagagtccttccgacgtcaagccactgccaagcccagataccgatgttccgctttcttcagtggagattgagaaccccgaaacttctgaccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 36 - protein, 418 aa, synthetic construct
  REGION 1..418: note = Description of Artificial Sequence: Synthetic polypeptide
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 37 - protein, 446 aa, synthetic construct
  REGION 1..446: note = Description of Artificial Sequence: Synthetic polypeptide
MYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRL

SEQ ID NO: 38 - protein, 479 aa, synthetic construct
  REGION 1..479: note = Description of Artificial Sequence: Synthetic polypeptide
MYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRLIKKSPSDVKPLPSPDTDVPLSSVEIENPETSDQ

SEQ ID NO: 39 - protein, 453 aa, synthetic construct
  REGION 1..453: note = Description of Artificial Sequence: Synthetic polypeptide
MYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRL

SEQ ID NO: 40 - protein, 486 aa, synthetic construct
  REGION 1..486: note = Description of Artificial Sequence: Synthetic polypeptide
MYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRLIKKSPSDVKPLPSPDTDVPLSSVEIENPETSDQ

SEQ ID NO: 41 - protein, 436 aa, synthetic construct
  REGION 1..436: note = Description of Artificial Sequence: Synthetic polypeptide
EGRGSLLTCGDVEENPGPMPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 42 - protein, 464 aa, synthetic construct
  REGION 1..464: note = Description of Artificial Sequence: Synthetic polypeptide
EGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRL

SEQ ID NO: 43 - protein, 497 aa, synthetic construct
  REGION 1..497: note = Description of Artificial Sequence: Synthetic polypeptide
EGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRLIKKSPSDVKPLPSPDTDVPLSSVEIENPETSDQ

SEQ ID NO: 44 - protein, 471 aa, synthetic construct
  REGION 1..471: note = Description of Artificial Sequence: Synthetic polypeptide
EGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRL

SEQ ID NO: 45 - protein, 504 aa, synthetic construct
  REGION 1..504: note = Description of Artificial Sequence: Synthetic polypeptide
EGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRLIKKSPSDVKPLPSPDTDVPLSSVEIENPETSDQ

SEQ ID NO: 46 - protein, 443 aa, synthetic construct
  REGION 1..443: note = Description of Artificial Sequence: Synthetic polypeptide
RAKRGSGEGRGSLLTCGDVEENPGPMPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 47 - protein, 471 aa, synthetic construct
  REGION 1..471: note = Description of Artificial Sequence: Synthetic polypeptide
RAKRGSGEGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRL

SEQ ID NO: 48 - protein, 504 aa, synthetic construct
  REGION 1..504: note = Description of Artificial Sequence: Synthetic polypeptide
RAKRGSGEGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGLIIFGVMAGVIGTILLISYGIRRLIKKSPSDVKPLPSPDTDVPLSSVEIENPETSDQ

SEQ ID NO: 49 - protein, 478 aa, synthetic construct
  REGION 1..478: note = Description of Artificial Sequence: Synthetic polypeptide
RAKRGSGEGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRL

SEQ ID NO: 50 - DNA, 20 bp, Homo sapiens
  source 1..20: mol_type = unassigned DNA
ggcaagaagcatggccaccg

SEQ ID NO: 51 - DNA, 20 bp, Homo sapiens
  source 1..20: mol_type = unassigned DNA
gcagcatagtgagcccagaa

SEQ ID NO: 52 - DNA, 945 bp, Homo sapiens
  source 1..945: mol_type = unassigned DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccac

SEQ ID NO: 53 - DNA, 879 bp, Homo sapiens
  source 1..879: mol_type = unassigned DNA
tggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 54 - DNA, 502 bp, Homo sapiens
  source 1..502: mol_type = unassigned DNA
gctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttc

SEQ ID NO: 55 - DNA, 500 bp, Homo sapiens
  source 1..500: mol_type = unassigned DNA
tgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 56 - DNA, 72 bp, Homo sapiens
  source 1..72: mol_type = unassigned DNA
ctgataatattcggcgttatggccggcgtgatcggtacaattctgctcatcagttatgggatacggagattg

SEQ ID NO: 57 - DNA, 102 bp, Homo sapiens
  source 1..102: mol_type = unassigned DNA
atcaagaagagtccttccgacgtcaagccactgccaagcccagataccgatgttccgctttcttcagtggagattgagaaccccgaaacttctgaccagtga

SEQ ID NO: 58 - DNA, 57 bp, Homo sapiens
  source 1..57: mol_type = unassigned DNA
atgtacgggaaaatcattttcgtcctgctgctttctgagatcgtttctatcagcgct

SEQ ID NO: 59 - DNA, 27 bp, synthetic construct
  misc_feature 1..27: note = Description of Artificial Sequence: Synthetic oligonucleotide
  source 1..27: mol_type = other DNA
ggatcaggcggatccggcggtagcggt

SEQ ID NO: 60 - DNA, 21 bp, synthetic construct
  misc_feature 1..21: note = Description of Artificial Sequence: Synthetic oligonucleotide
  source 1..21: mol_type = other DNA
ccacttggtatgtggtctagg

SEQ ID NO: 61 - DNA, 500 bp, Homo sapiens
  source 1..500: mol_type = unassigned DNA
gaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcagg

SEQ ID NO: 62 - DNA, 1257 bp, Homo sapiens
  source 1..1257: mol_type = unassigned DNA
atgccttcatcagtatcttggggaatactgctccttgctgggttgtgttgtctcgtacccgtgagtctcgccgaagaccctcaaggcgacgccgcacaaaagactgacacttctcatcacgaccaagaccatcctacatttaataaaattactccaaatctcgccgaatttgcgttttctctgtataggcaactcgctcaccaatctaattcaacgaacatattcttttcacctgtttccatagccaccgctttcgccatgctgagtttgggaacaaaagcagatacccatgacgagatactcgaaggactcaactttaatctgacagaaatccctgaagcacaaattcacgagggttttcaagagctgctgagaactttgaatcaacccgattcccaattgcaactcacaacaggaaacggtttgtttctttcagaagggctcaaactggtcgacaaattcctcgaagacgtgaagaaactttatcatagcgaggcttttaccgtgaattttggagatacggaagaagctaagaagcaaataaatgactatgtcgaaaaggggacacagggaaagatagttgacctggtgaaagaactggatagggatactgtgttcgcgctcgtcaactatatcttcttcaaggggaagtgggaacggccattcgaggttaaagatacagaagaggaagattttcatgtagatcaagtcacaacagtcaaagttccaatgatgaaacgcctcgggatgttcaatatacaacattgcaagaaacttagctcatgggtccttttgatgaagtatctcgggaacgctacagcgatattctttctcccagacgaaggtaagctgcaacatcttgagaacgagctgacacatgacataataacaaaatttcttgagaacgaggatcgccggtccgcatccctgcacctgccgaagcttagcataaccggcacatacgacttgaaatctgttcttgggcagcttggtattacaaaagtgttttccaacggcgcggatctgtcaggcgtgacggaagaagctcctcttaaactgagtaaagcagtccacaaagcagtactcactattgatgaaaagggtaccgaggcggccggagctatgttcctcgaagctattcctatgagtattccccctgaagttaaatttaataagcctttcgtgtttctcatgatagagcagaacacgaaaagccctctgtttatgggcaaggtcgtcaacccaacacagaagtaa

SEQ ID NO: 63 - protein, 24 aa, Homo sapiens
LIIFGVMAGVIGTILLISYGIRRL

SEQ ID NO: 64 - protein, 33 aa, Homo sapiens
IKKSPSDVKPLPSPDTDVPLSSVEIENPETSDQ

SEQ ID NO: 65 - protein, 19 aa, Homo sapiens
MYGKIIFVLLLSEIVSISA

SEQ ID NO: 66 - protein, 9 aa, synthetic construct
  REGION 1..9: note = Description of Artificial Sequence: Synthetic peptide
GSGGSGGSG

SEQ ID NO: 67 - protein, 7 aa, synthetic construct
  REGION 1..7: note = Description of Artificial Sequence: Synthetic peptide
PLGMWSR

SEQ ID NO: 68 - protein, 418 aa, Homo sapiens; identical to SEQ ID NO: 36

SEQ ID NO: 69 - protein, 511 aa, synthetic construct
  REGION 1..511: note = Description of Artificial Sequence: Synthetic polypeptide
RAKRGSGEGRGSLLTCGDVEENPGPMYGKIIFVLLLSEIVSISAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSGGSGGSGPLGMWSRLIIFGVMAGVIGTILLISYGIRRLIKKSPSDVKPLPSPDTDVPLSSVEIENPETSDQ

(19) United States
(12) Patent Application Publication
(10) Pub. No.: US 2024/0156873 A1
(43) Pub. Date: May 16, 2024

(54) METHODS TO GENETICALLY ENGINEER HEMATOPOIETIC STEM AND PROGENITOR CELLS FOR RED CELL SPECIFIC EXPRESSION OF THERAPEUTIC PROTEINS

(71) Applicant: Graphite Bio, Inc., South San Francisco, CA (US)

(72) Inventors: Beeke WIENERT, South San Francisco, CA (US); Daniel DEVER, South San Francisco, CA (US); Aishwarya CHURI, South San Francisco, CA (US)

(21) Appl. No.: 18/532,004

(22) Filed: Dec. 7, 2023

Related U.S. Application Data
(63) Continuation of application No. PCT/US2022/033487, filed on Jun. 14, 2022.
(60) Provisional application No. 63/210,298, filed on Jun. 14, 2021.

(51) Int. Cl. A61K 35/28; C07K 14/47; C07K 14/81; C12N 5/0789; C12N 9/22; C12N 15/11; C12N 15/86

(52) U.S. Cl. CPC A61K 35/28; C07K 14/47; C07K 14/8125; C12N 5/0647; C12N 9/22; C12N 15/11; C12N 15/86; C07K 2319/03; C12N 2310/20; C12N 2750/14143

(57) ABSTRACT

Provided herein are compositions, methods, and systems, comprising a programmable nucleic acid-guided nuclease and donor polynucleotides sequences to introduce an exogenous polynucleotide sequence, which encodes a therapeutic protein linked to a transmembrane domain. The composition of the disclosure is introduced into a cell, such as an HSPC, wherein the HSPC can be further differentiated.

### CROSS REFERENCE TO RELATED APPLICATIONS

This application is a continuation of International Application No. PCT/US2022/033487 filed on Jun. 14, 2022 which claims the benefit of, and priority to, U.S. provisional patent application Ser. No. 63/210,298, filed on Jun. 14, 2021, each of which is hereby incorporated by reference herein in its entirety.

### SEQUENCE LISTING

The instant application contains a Sequence Listing which has been submitted electronically in XML format and is hereby incorporated by reference in its entirety. Said XML copy, created on Dec. 6, 2023 is named 66874_702_301_SL.xml and is 202,198 bytes in size.

### SUMMARY OF THE INVENTION

The present disclosure provides a method of expressing an exogenous protein of interest in a cell, the method comprising introducing into the cell: i) a programmable nucleic acid-guided nuclease and an engineered guide polynucleotide, wherein the guide polynucleotide hybridizes to a target sequence in an endogenous gene; and ii) a donor polynucleotide sequence comprising: a) an exogenous polynucleotide sequence encoding at least one therapeutic protein and a transmembrane domain, wherein the at least one therapeutic protein and the transmembrane domain are operably linked by a linker; and b) 5′ homology and 3′ homology arms flanking the exogenous polynucleotide sequence, wherein the homology arms are homologous to portions of the endogenous gene. Generation of a double-strand break within the target sequence by the programmable nucleic acid-guided nuclease results in integration of the donor polynucleotide sequence into the endogenous gene locus by homology directed repair (HDR).

The present disclosure provides one embodiment comprising a method of expressing an exogenous protein of interest in a cell, the method comprising introducing into the cell: i) a programmable nucleic acid-guided nuclease and an engineered guide polynucleotide, wherein the guide polynucleotide hybridizes to a target sequence in an endogenous gene; ii) a donor polynucleotide sequence comprising: a) the exogenous polynucleotide sequence encoding at least one therapeutic protein and a transmembrane domain, and wherein the at least one therapeutic protein and the transmembrane domain are operably linked by a cleavable linker; and b) a 5′ homology and 3′ homology arm flanking the exogenous polynucleotide sequence, wherein the homology arms are homologous to a portion of the endogenous gene.

The present disclosure also provides a method of expressing an exogenous protein of interest in a cell, the method comprising introducing into the cell: i) a programmable nucleic acid-guided nuclease and an engineered guide polynucleotide, wherein the guide polynucleotide hybridizes to a target sequence in the HBA1 gene; ii) a donor polynucleotide sequence comprising: a) the exogenous polynucleotide sequence encoding at least one therapeutic protein and a transmembrane domain, wherein the therapeutic protein and the transmembrane domain are operably linked by a linker; and b) a 5′ homology and 3′ homology arm flanking the exogenous polynucleotide sequence, wherein the homology arms are homologous to at least a portion of the HBA1/2 gene.

The present disclosure also provides a method of expressing an exogenous protein of interest in a cell, the method comprising introducing into the cell: i) a programmable nucleic acid-guided nuclease and an engineered guide polynucleotide, wherein the guide polynucleotide hybridizes to a target sequence in the CCR5 locus; ii) a donor polynucleotide sequence comprising: a) the exogenous polynucleotide sequence encoding at least one therapeutic protein and a transmembrane domain; and b) a 5′ homology and 3′ homology arm flanking the exogenous polynucleotide sequence; and wherein the homology arms are homologous to at least a portion of the CCR5 locus.

In some embodiments, the endogenous gene is the HBA1 gene. In some embodiments, the endogenous gene is the CCR5 gene.

In some embodiments, the programmable nuclease is a CRISPR-associated Cas protein. In some embodiments, the programmable nuclease is selected from the group consisting of Cas9, Cpf1, or any functional variant thereof. In some embodiments, the Cas9 is a high-fidelity Cas9. In some embodiments, the Cas9 comprises a mutation at position R691. In some embodiments, the mutation at position R691 is an alanine.

In some embodiments, the target gene comprises a safe harbor site. In some embodiments, the safe harbor site is selected from the group consisting of: HBA1, HBA2, CCR5 locus, AAVS1, the human ortholog of the murine Rosa26 locus.

In some embodiments, the engineered guide polynucleotide sequence comprises at hybridizes to a sequence having at least 75% sequence identity to SEQ ID NO: 1 or SEQ ID NO: 2. In some embodiments, the engineered guide polynucleotide sequence comprises at hybridizes to a sequence having at least 75% sequence identity to SEQ ID NO: 1. In some embodiments, the engineered guide polynucleotide sequence comprises at hybridizes to a sequence having at least 75% sequence identity to SEQ ID NO: 2.

In some embodiments, the linker is a cleavable linker or a non-cleavable linker.

In some embodiments, the cleavable linker comprises at least one recognition motif for a protease. In some embodiments, the protease is selected from the group consisting of: metalloproteases, Serine proteases, Cysteine proteases, threonine proteases, Aspartic proteases, Glutamic proteases and Asparagine proteases.

In some embodiments, the linker is a matrix metalloproteinase (MMP) linker. In some embodiments, the therapeutic protein comprises alpha-antitrypsin (AAT) or an active variant or portion thereof.

In some embodiments, the non-cleavable linker comprises SEQ ID NO: 60 encoding SEQ ID NO: 67.

In some embodiments, the exogenous polynucleotide sequence encoding the therapeutic protein comprises polynucleotide sequence having at least a portion of alpha-antitrypsin. In some embodiments, the therapeutic protein comprises a polynucleotide sequence having at least 75% sequence homology to SEQ ID NO: 62.

In some embodiments, the exogenous polynucleotide further comprises an exogenous promoter sequence. In some embodiments, the promoter sequence comprises a polynucleotide sequence having at least 75% sequence identity to SEQ ID NO: 61.

In some embodiments, the exogenous polynucleotide sequence encoding the transmembrane domain comprises a glycophorin A (GPA) transmembrane domain. In some embodiments, the exogenous polynucleotide sequence encoding the transmembrane domain has at least 75% sequence identity to SEQ ID NO: 56. In some embodiments, the transmembrane domain comprises a polypeptide sequence having at least 75% sequence homology to SEQ ID NO::63. In some embodiments, the exogenous polynucleotide sequence further comprises a C-terminal tail.

In some embodiments, the C-terminal tail comprises a polynucleotide sequence having at least 75% sequence identity SEQ ID NO: 57. In some embodiments, the C-terminal tail comprises a polypeptide sequence having at least 75% sequence homology to SEQ ID NO: 64. In some embodiments, the 5′ and 3′ homology arms comprise at least a portion of HBA1.

In some embodiments, the 5′ homology and 3′ homology arms comprise a polynucleotide sequence having at least 75% sequence identity to SEQ ID NO: 52 and SEQ ID NO: 53, respectively. In some embodiments, the 5′ and 3′ homology arms comprise at least a portion of CCR5.

In some embodiments, the 5′ homology and 3′ homology arms comprise a polynucleotide sequence having at least 75% sequence identity to SEQ ID NO: 54 and SEQ ID NO: 55, respectively. In some embodiments, the donor polynucleotide is arranged from 5′ to 3′ in any one of the following ways: a) 5′ homology arm-promoter-therapeutic protein-cleavable linker-GPA-GPA(C-term)-3′ homology arm; b) 5′ homology arm-promoter-therapeutic protein-non cleavable linker-GPA-GPA(C-term)-3′ homology arm; c) 5′ homology arm-promoter-therapeutic protein-cleavable linker-GPA-3′ homology arm; d) 5′ homology arm-promoter-therapeutic protein-non cleavable linker-GPA-3′ homology arm; e) 5′ homology arm-therapeutic protein-cleavable linker-GPA-GPA(C-term)-3′ homology arm; f) 5′ homology arm-therapeutic protein-non cleavable linker-GPA-GPA(C-term)-3′ homology arm; g) 5′ homology arm-therapeutic protein-cleavable linker-GPA-3′ homology arm; or h) 5′ homology arm-therapeutic protein-non cleavable linker-GPA-3′ homology arm. In some embodiments, the donor polynucleotide sequence comprises a polynucleotide sequence having at least 75% sequence identity to a sequence selected from the group consisting of SEQ ID NO: 1-SEQ ID NO: 35. In some embodiments, the donor polynucleotide sequence comprises a polynucleotide sequence which encodes a polypeptide sequence having at least 75% sequence homology to SEQ ID NO: 36-SEQ ID NO: 49, and SEQ ID NO: 69.

In some embodiments, the cell is an HSPC. In some embodiments, the HSPC is further differentiated into an erythrocyte.

The present disclosure provides a genetically modified HSPC, prepared according to the method of the present disclosure. In some embodiments, the HSPC expresses a polypeptide comprising a transmembrane domain and a therapeutic protein, wherein the transmembrane domain and therapeutic protein are operably linked by a linker. In some embodiments, the genetically modified HSPC can be further differentiated into an erythrocyte.

The present disclosure provides an exogenous protein expression system comprising the cell of the present disclosure.

The present disclosure provides an exogenous protein cell expression kit, comprising the method of the present disclosure.

The present disclosure provides an AAV vector comprising: a donor polynucleotide sequences comprising: an exogenous polynucleotide sequence encoding a transmembrane domain and a therapeutic protein; and 5′ and 3′ homology arms flanking the exogenous polynucleotide sequence wherein the homology arms are homologous to a portion of an endogenous gene.

In some embodiments, the therapeutic protein and the transmembrane domain are operably linked. In some embodiments, the linker is a cleavable linker or a non-cleavable linker. In some embodiments, the cleavable linker comprises at least one recognition motif for a protease. In some embodiments, the protease is selected from the group consisting of: metalloproteases, Serine proteases, Cysteine proteases, Threonine proteases, Aspartic proteases, Glutamic proteases and Asparagine proteases.

In some embodiments, the non-cleavable linker comprises SEQ ID NO: 59, which encodes SEQ ID NO: 66.

In some embodiments, the exogenous polynucleotide sequence encoding the therapeutic protein comprises polynucleotide sequence having at least a portion of alpha-antitrypsin.

In some embodiments, the therapeutic protein comprises a polynucleotide sequence having at least 75% sequence identity to SEQ ID NO: 62. In some embodiments, the exogenous polynucleotide further comprises an exogenous promoter sequence. In some embodiments, the promoter sequence comprises a polynucleotide sequence having at least 75% sequence identity to SEQ ID NO: 61. In some embodiments, the exogenous polynucleotide sequence encoding the transmembrane domain comprises a glycophorin A (GPA) transmembrane domain. The method of claim 53, wherein the exogenous polynucleotide sequence encoding the transmembrane domain has at least 75% sequence identity to SEQ ID NO: 56.

In some embodiments, the transmembrane domain comprises a polypeptide sequence having at least 75% sequence homology to SEQ ID NO: 63.

In some embodiments, the exogenous polynucleotide sequence further comprises a C-terminal tail.

In some embodiments, the C-terminal tail comprises a polynucleotide sequence having at least 75% sequence identity SEQ ID NO: 57.

In some embodiments, the C-terminal tail comprises a polypeptide sequence having at least 75% sequence homology to SEQ ID NO: 64.

In some embodiments, the 5′ and 3′ homology arms comprise at least a portion of HBA1.

In some embodiments, the 5′ homology and 3′ homology arms comprise a polynucleotide sequence having at least 75% sequence identity to SEQ ID NO: 52 and SEQ ID NO: 53, respectively.

In some embodiments, the 5′ and 3′ homology arms comprise at least a portion of CCR5.

In some embodiments, the 5′ homology and 3′ homology arms comprise a polynucleotide sequence having at least 75% sequence identity to SEQ ID NO: 54 and SEQ ID NO: 55, respectively.

In some embodiments, the donor polynucleotide can be arranged from 5′ to 3′ in any one of the following ways: a) 5′ homology arm-promoter-therapeutic protein-cleavable linker-GPA-GPA(C-term)-3′ homology arm; b) 5′ homology arm-promoter-therapeutic protein-non cleavable linker-GPA-GPA(C-term)-3′ homology arm; c) 5′ homology arm-promoter-therapeutic protein-cleavable linker-GPA-3′ homology arm; d) 5′ homology arm-promoter-therapeutic protein-non cleavable linker-GPA-3′ homology arm; e) 5′ homology arm-therapeutic protein-cleavable linker-GPA-GPA(C-term)-3′ homology arm; f) 5′ homology arm-therapeutic protein-non cleavable linker-GPA-GPA(C-term)-3′ homology arm; g) 5′ homology arm-therapeutic protein-cleavable linker-GPA-3′ homology arm; or h) 5′ homology arm-therapeutic protein-non cleavable linker-GPA-3′ homology arm.

In some embodiments, the donor polynucleotide sequence comprises a polynucleotide sequence having at least 75% sequence identity to a sequence selected from the group consisting of SEQ ID NO: 1-SEQ ID NO: 35.

In some embodiments, the donor polynucleotide sequence comprises a polynucleotide sequence which encodes a polypeptide sequence having at least 75% sequence homology to SEQ ID NO: 36-SEQ ID NO: 49, and SEQ ID NO: 69.

Provided herein, the present disclosure provides a method of treating alpha-antitrypsin deficiency in a subject in need thereof, the method comprising: i) introducing into an HSPC a nucleic acid-guide programmable nuclease and an engineered guide polynucleotide comprising a sequence selected from the group consisting of SEQ ID NO: 51 or SEQ ID NO: 52, wherein the engineered guide polynucleotide hybridizes to a target gene; ii) introducing a recombinant AAV6 vector comprising a donor polynucleotide sequence into the HSPC, wherein the donor polynucleotide comprises an exogenous polynucleotide sequence comprising a sequence selected from the group consisting of: NO 1 to SEQ ID NO: 35, wherein the exogenous polynucleotide sequence inserts itself within the target gene of the HSPC through a single recombination event; thereby generating a genetically modified HSPC; and iii) introducing the genetically modified HSPC into the subject, thereby treating the AAT deficiency in the subject.

Provided herein, the present disclosure provides a donor polynucleotide comprising a sequence having at least 70% sequence identity to SEQ ID NO: 1 to SEQ ID NO: 35.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-FIG. 1D show exemplary strategies for targeted gene insertion into safe harbor loci, and a schematic of protein expression on the surface of cells engineered with said strategy. FIG. 1A shows insertion into a safe harbor locus, alpha-hemoglobin (HBA1). The dotted lines denote sites of homology, thereby facilitating homologous recombination and inserting the gene construct into the target locus. FIG. 1B shows a similar targeting strategy using another safe harbor locus, C—C chemokine receptor Type 5 (CCR5). Sign—signaling peptide of either GOI or GPA; GOI—gene of interest; TM GPA—transmembrane domain of GPA; C-term GPA—full C-terminus of GPA protein; prom.—red blood cell specific promoter. FIG. 1C shows a targeting strategy that is specific for exon 3 of HBA1. Sign—signaling peptide of either GOI or GPA; GOI—gene of interest; TM GPA—transmembrane domain of GPA; C-term GPA—full C-terminus of GPA protein. FIG. 1D shows a targeting strategy that is specific for exon 3 of HBA1. Sign—signaling peptide of either GOI or GPA; GOI—gene of interest; TM GPA—transmembrane domain of GPA; C-term GPA—full C-terminus of GPA protein; furin—furin cleavage site.

FIG. 2 shows a schematic of a red blood cell expressing a protein of interest (POI) on the cell surface. The POI is anchored to the cell membrane by a linker fused to the transmembrane domain of glycophorin A (GPA) and the C-terminus of GPA (GPA C-term). When the cell is in the presence of a protease specific for the linker, the POI is cleaved off the cell and released into the extracellular space where it may become active.

FIGS. 3A-FIG. 3D demonstrates that proteins fused to the cell surface of cells can be cleaved by proteases present in the cell culture media. FIG. 3A shows a schematic of constructs used in HEK293 cells. Ef1a—Ef1a promoter; AAT CDS—coding sequence of alpha-anti trypsin (AAT); GS—glycine/serine linker; pA—polyA tail; myc—3×myc peptide tag; MMP—consensus cleavage site for matrix metalloproteinase 9. FIG. 3B shows Western Blots demonstrating expression of constructs in HEK293 cells. Blots were probed with either an anti-myc antibody (left) or an anti-AAT antibody (right). FIG. 3C shows flow cytometry data of HEK293 cells expressing the constructs outlined in FIG. 3A. Cells were stained on the cell surface with either myc (top panel) or AAT antibody (bottom panel). FIG. 3D shows a Western Blot demonstrating the presence of AAT protein in cell extracts (left) or in the growth media of HEK293 cells expressing constructs ii-iv of FIG. 3A. Blots were probed with an anti-myc antibody.

### DETAILED DESCRIPTION OF THE INVENTION

Provided herein are methods and compositions to introduce a donor polynucleotide comprising coding sequences for a therapeutic protein into a cell, for example, a hematopoietic stem and progenitor cell (HSPC), which, in some embodiments, can be differentiated into an erythrocyte. The donor polynucleotide can further include coding sequences which, when expressed as part of the therapeutic protein, can direct expression of the therapeutic protein to the surface of the cell, for example, the surface of a differentiated erythrocyte derived from an HSPC genetically modified to comprise the donor polynucleotide. In further embodiments, the therapeutic protein can be operably linked to a transmembrane domain, which localizes the therapeutic protein to the surface of the cell, through a linker, which may be non-cleavable or cleavable to release the therapeutic protein from the surface of the cell.

Methods of treatments and compositions are described herein and are directed to the treatment of anti-alpha trypsin but can be broadly expanded to other diseases or disorder where treatment is amenable to the composition described herein. The present disclosure describes the use of CRISPR-Cas9 to introduce a double stranded break into a safe harbor locus, for example, the HBA1 or CCR5 gene, to facilitate integration of a donor polynucleotide sequence comprising the gene of interest. The gene of interest encodes a therapeutic protein that is linked to a transmembrane domain using a cleavable or non-cleavable linker. The gene of interest can be flanked by regions of homology, or homology arms, allowing for targeted integration of the donor polynucleotide directed by homology directed recombination (HDR).

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “subject” as used herein, refers to a mammal (e.g., a human).

The term “administering” as used herein refers to a method of giving a dosage of an antibody or fragment thereof, or a composition (e.g., a pharmaceutical composition) to a subject. The method of administration can vary depending on various factors (e.g., the binding protein or the pharmaceutical composition being administered, and the severity of the condition, disease, or disorder being treated).

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

By “donor polynucleotide,” the present disclosure refers to a polynucleotide sequence comprising a gene sequence (including, for example, coding and non-coding regulatory sequences) that is flanked by a 5′ and 3′ homology arm that is complementary to the gene that is to be replaced. The donor polynucleotide can be a circular plasmid, linear, or made to be linear through a cleavage process.

A “Cas9 molecule,” as used herein, refers to a Cas9 polypeptide or a nucleic acid encoding a Cas9 polypeptide. A “Cas9 polypeptide” is a polypeptide that can interact with a gRNA molecule and, in concert with the gRNA molecule, localize to a site comprising a target domain and, in certain embodiments, a PAM sequence. Cas9 molecules include both naturally occurring Cas9 molecules and Cas9 molecules and engineered, altered, or modified Cas9 molecules or Cas9 polypeptides that differ, e.g., by at least one amino acid residue, from a reference sequence, e.g., the most similar naturally occurring Cas9 molecule. (The terms altered, engineered or modified, as used in this context, refer merely to a difference from a reference or naturally occurring sequence, and impose no specific process or origin limitations.) A Cas9 molecule may be a Cas9 polypeptide or a nucleic acid encoding a Cas9 polypeptide. A Cas9 molecule may be a nuclease (an enzyme that cleaves both strands of a double-stranded nucleic acid), a nickase (an enzyme that cleaves one strand of a double-stranded nucleic acid), or an enzymatically inactive (or dead) Cas9 molecule. A Cas9 molecule having nuclease or nickase activity is referred to as an “enzymatically active Cas9 molecule” (an “eaCas9” molecule). A Cas9 molecule lacking the ability to cleave target nucleic acid is referred to as an “enzymatically inactive Cas9 molecule” (an “eiCas9” molecule). Cas molecule. Exemplary Cas molecules include high-fidelity Cas variants having improved on-target specificity and reduced off-target activity. Examples of high-fidelity Cas9 variants include but are not limited to those described in PCT Publication Nos. WO/2018/068053 and WO/2019/074542, each of which is herein incorporated by reference in its entirety.

As used herein, the term “gRNA molecule” or “gRNA” refers to a guide RNA which is capable of targeting a Cas molecule to a target nucleic acid. In one embodiment, the term “gRNA molecule” refers to a guide ribonucleic acid. In another embodiment, the term “gRNA molecule” refers to a nucleic acid encoding a gRNA. In one embodiment, a gRNA molecule is non-naturally occurring. In one embodiment, a gRNA molecule is a synthetic gRNA molecule.

“HDR”, or “homology-directed repair,” as used herein, refers to the process of repairing DNA damage using a homologous nucleic acid (e.g., an endogenous homologous sequence, e.g., a sister chromatid, or an exogenous nucleic acid, e.g., a template nucleic acid such as a donor polynucleotide described herein). Canonical HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded. This process is used by a number of site-specific nuclease systems that create a double-strand break, such as meganucleases, zinc finger nucleases (ZFNs), transcription activator-like effector nucleases (TALENs), and the CRISPR-Cas gene editing systems. In particular embodiments, HDR involves double-stranded breaks induced by CRISPR-Cas nuclease, e.g. Cas9.

As used herein, “operably linked” refers to a functional linkage between nucleic acid sequences such that the sequences encode a desired function. For example, a coding sequence for a gene of interest, e.g., a therapeutic protein, is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in host cell.

### Targeted Gene Insertion

The present disclosure provides compositions and methods for introducing a portion of an exogenous polynucleotide sequence into a target site of an endogenous polynucleotide sequence.

CRISPR-Cas9 systems are quickly emerging as an attractive tool to introduce double stranded breaks. Briefly, CRISPR-Cas9 systems utilize a guide RNA or guide polynucleotide to guide the Cas9 nuclease to a target site to introduce a double stranded break into the sequence.

A donor template or donor polynucleotide sequence can be used simultaneously to utilize HDR machinery that can resect the donor polynucleotide sequence into the endogenous sequence through the regions of the donor polynucleotide having high homology or sequence identity. In this manner, targeted gene insertion can be performed by administering a nucleic acid guided programmable nuclease in combination with a donor polynucleotide.

In embodiments, the donor polynucleotide comprises an exogenous sequence that is flanked by regions containing high homology with the endogenous target locus or gene.

In some embodiments, the targeted gene insertion can replace at least a portion of the endogenous polynucleotide sequence.

Endogenous polynucleotides may contain polymorphisms or mutations that cause expression of an aberrant protein that results in the manifestation of a disease, such as alpha-antitrypsin deficiency. In some embodiments, the endogenous polynucleotide sequence comprises mutations, including but are not limited to missense and non-sense mutations. In some embodiments, the endogenous polynucleotide sequence can comprise insertions, deletions, or truncations.

### Donor Polynucleotide

The donor polynucleotide can comprise an exogenous polynucleotide sequence that replaces an endogenous sequence in a cell. The exogenous polynucleotide can comprise homology arms flanking the 5′ and 3′ ends of the exogenous polynucleotide sequence. The homology arms can be homologous to at least a portion of a safe harbor site. The homology arms can be homologous to at least a portion of a safe harbor site, such as the CCR5 or HBA1 locus.

The homology arms can be of variable lengths. In some embodiments, the 5′ and 3′ homology arms can be identical in length. In some embodiments the 5′ and 3′ homology arms can be different lengths.

In some embodiments, the 5′ homology arm comprises about 50 base pairs to about 1,000 base pairs. In some embodiments, the 5′ homology arm comprises at least about 50 base pairs. In some embodiments, the 5′ homology arm comprises at most about 1,000 base pairs. In some embodiments, the 5′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

### 5′ Homology Arm

In some embodiments, the 5′ homology arm comprises at least 60% sequence identity to a sequence selected from the group consisting of SEQ ID NO: 52 and SEQ ID NO: 54. In some embodiments, the 5′ homology arm comprises about 60% to about 99% to a sequence selected from the group consisting of SEQ ID NO: 52 and SEQ ID NO: 54. In some embodiments, the 5′ homology arm comprises at least 60% sequence identity to a sequence selected from the group consisting of SEQ ID NO: 52 and SEQ ID NO: 54. In some embodiments, the 5′ homology arm comprises at least 99% sequence identity to a sequence selected from the group consisting of SEQ ID NO: 52 and SEQ ID NO: 54. In some embodiments, the 5′ homology arm comprises 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 52 and SEQ ID NO: 54.

### 3′ Homology Arm

In some embodiments, the 3′ homology arm comprises about 50 base pairs to about 1,000 base pairs. In some embodiments, the 3′ homology arm comprises at least about 50 base pairs. In some embodiments, the 3′ homology arm comprises at most about 1,000 base pairs. In some embodiments, the 3′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

In some embodiments, the 3′ homology arm comprises at least 60% sequence identity to a sequence selected from the group consisting of SEQ ID NO: 53 and SEQ ID NO: 55. In some embodiments, the 3′ homology arm comprises about 60% to about 99% to a sequence selected from the group consisting of SEQ ID NO: 53 and SEQ ID NO: 55. In some embodiments, the 3′ homology arm comprises at least 60% sequence identity to a sequence selected from the group consisting of SEQ ID NO: 53 and SEQ ID NO: 55. In some embodiments, the 3′ homology arm comprises at least 99% sequence identity to a sequence selected from the group consisting of SEQ ID NO: 53 and SEQ ID NO: 55. In some embodiments, the 3′ homology arm comprises 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 53 and SEQ ID NO: 55.

### Therapeutic Protein

The present disclosure provides a donor polynucleotide comprises an exogenous polynucleotide sequence comprising a polynucleotide sequence that encodes for a therapeutic protein. The therapeutic protein can be any protein in which the presence of the protein can ameliorate symptoms of a disease or disorder. The therapeutic protein can be, but is not limited to, alpha-1 anti-trypsin. An exemplary, non-limiting list of suitable therapeutic proteins includes but is not limited to PDFGB (Platelet-derived growth factor subunit B; see, e.g., NCBI Gene ID No. 5155), IDUA (alpha-L-iduronidase; see, e.g., NCBI Gene ID No. 3425), PAH (phenylalanine hydroxylase; see, e.g., NCBI Gene ID No. 5053), LDLR (low density lipoprotein receptor; see, e.g., NCBI Gene ID No. 3949), cytokines, in particular interferon, more particularly interferon-alpha, interferon-beta or interferon-pi; hormones; chemokines; antibodies (including nanobodies); anti-angiogenic factors; enzymes for replacement therapy, such as for example adenosine deaminase, alpha glucosidase, alpha-galactosidase, alpha-L-iduronidase (also name idua) and beta-glucosidase; interleukins; insulin; G-CSF; GM-CSF; hPG-CSF; M-CSF; blood clotting factors such as Factor VIII, tPA or Factor IX (or FIX; see, e.g., NCBI Gene ID NO. 2158), including Hyperactive Factor DC Padua, or the Padua Variant (see, e.g., Simioni et al., (2009) NEJM 361:1671-1675; Cantore et al. (2012) Blood 120:4517-4520; Monahan et al., (2015) Hum. Gene. Ther. 26:69-81); transmembrane proteins such as Nerve Growth Factor Receptor (NGFR); lysosomal enzymes such as a-galactosidase (GLA), a-L-iduronidase (IDUA), lysosomal acid lipase (LAL) and galactosamine (N-acetyl)-6-sulfatase (GALNS); any protein that can be engineered to be secreted and eventually uptaken by non-modified cells (for example Lawlor M W, Hum Mol Genet. 22(8): 1525-1538. (2013); Puzzo F, Sci Transl Med. 29; 9(418) (2017); Bolhassani A. Peptides. 87:50-63, (2017)) and combinations thereof, and preferably is a blood clotting factor, more preferably Factor VIII; or a lysosomal enzyme, in particular lysosomal acid lipase (LAL) or galactosamine (N-acetyl)-6-sulfatase (GALNS).

In some embodiments, the polynucleotide sequence coding the therapeutic protein comprises at least 60% sequence identity to a sequence selected from the group consisting of SEQ ID NO: 62. In some embodiments, the polynucleotide sequence coding the therapeutic protein comprises about 60% to about 99% to a sequence selected from the group consisting of SEQ ID NO: 62. In some embodiments, the polynucleotide sequence coding the therapeutic protein comprises at least 60% sequence identity to a sequence selected from the group consisting of SEQ ID NO: 62. In some embodiments, the polynucleotide sequence coding the therapeutic protein comprises at least 99% sequence identity to a sequence selected from the group consisting of SEQ ID NO: 62. In some embodiments, the polynucleotide sequence coding the therapeutic protein comprises 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 62.

In some embodiments, the therapeutic protein comprises an amino acid sequence having at least 60% sequence identity to a sequence selected from the group consisting of SEQ ID NO: 68. In some embodiments, the therapeutic protein comprises an amino acid sequence having about 60% to about 99% to a sequence selected from the group consisting of SEQ ID NO: 68. In some embodiments, the therapeutic protein comprises an amino acid sequence having at least 60% sequence identity to a sequence selected from the group consisting of SEQ ID NO: 68. In some embodiments, the therapeutic protein comprises an amino acid sequence having at least 99% sequence identity to a sequence selected from the group consisting of SEQ ID NO: 68. In some embodiments, the therapeutic protein comprises an amino acid sequence having 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 68.

The therapeutic protein can be a pro-protein that is activated by a biochemical process, such as proteolytic cleavage. In some embodiments, the therapeutic protein is expressed in its inactive form. Upon contact with the appropriate protease, the therapeutic protein becomes activated and can carry out its function within a cell or subject.

### Transmembrane Protein

The therapeutic protein of the present disclosure can be linked to a transmembrane domain of a protein. The transmembrane can include a C-terminal tail. In some embodiments, the therapeutic protein is linked to the transmembrane domain. The transmembrane domain can be at least a portion of glycophorin A (GPA) and can optionally include a C-terminal tail of GPA.

In some embodiments, the polynucleotide sequence encoding the transmembrane domain comprises at least 60% sequence identity to a sequence selected from the group consisting of SEQ ID NO: 56 and SEQ ID NO: 57. In some embodiments, the polynucleotide sequence coding the transmembrane domain comprises about 60% to about 99% to a sequence selected from the group consisting of SEQ ID NO: 56 and SEQ ID NO: 57. In some embodiments, the polynucleotide sequence coding the transmembrane domain comprises at least 60% sequence identity to a sequence selected from the group consisting of SEQ ID NO: 56 and SEQ ID NO: 57. In some embodiments, the polynucleotide sequence coding the transmembrane domain comprises at least 99% sequence identity to a sequence selected from the group consisting of SEQ ID NO: 56 and SEQ ID NO: 57. In some embodiments, the polynucleotide sequence coding the transmembrane domain comprises 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 56 and SEQ ID NO: 57.

In some embodiments, the transmembrane domain comprises an amino acid sequence having at least 60% sequence identity to a sequence selected from the group consisting of SEQ ID NO: 63 and SEQ ID NO: 64. In some embodiments, the transmembrane domain comprises an amino acid sequence having about 60% to about 99% to a sequence selected from the group consisting of SEQ ID NO: 63 and SEQ ID NO: 64. In some embodiments, the transmembrane domain comprises an amino acid sequence having at least 60% sequence identity to a sequence selected from the group consisting of SEQ ID NO: 63 and SEQ ID NO: 64. In some embodiments, the transmembrane domain comprises an amino acid sequence having at least 99% sequence identity to a sequence selected from the group consisting of SEQ ID NO: 63 and SEQ ID NO: 64. In some embodiments, the transmembrane domain comprises an amino acid sequence having 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 63 and SEQ ID NO: 64.

### Donor Polynucleotide

In some embodiments, the donor polynucleotide comprises at least 60% sequence identity to a sequence selected from the group consisting of SEQ ID NO: 1-SEQ ID NO: 35. In some embodiments, the donor polynucleotide comprises about 60% to about 99% to a sequence selected from the group consisting of SEQ ID NO: 1-SEQ ID NO: 35. In some embodiments, the donor polynucleotide comprises at least 60% sequence identity to a sequence selected from the group consisting of SEQ ID NO: 1-SEQ ID NO: 35. In some embodiments, the donor polynucleotide comprises at least 99% sequence identity to a sequence selected from the group consisting of SEQ ID NO: 1-SEQ ID NO: 35. In some embodiments, the donor polynucleotide comprises 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% to a sequence selected from the group consisting of SEQ ID NO: 1-SEQ ID NO: 35.

In some embodiments, the donor polynucleotide comprises at least 70% sequence identity to SEQ ID NO: 1-SEQ ID NO: 35.

### Linkers

Polypeptide compositions and polynucleotides encoding the polypeptide compositions are described herein, in which the polypeptide compositions comprise a first and second peptide/polypeptide, connected by a linker sequence disclosed herein. In some embodiments, the first polypeptide comprises a therapeutic protein and the second polypeptide comprises a transmembrane domain. In some embodiments, of the present disclosure, the therapeutic protein and the transmembrane domain are operably linked by a linker sequence.

In some embodiments, the linker sequence can be non-cleavable linker. In some embodiments, the linker sequence is encoded by a polynucleotide sequence having at least 75%, 80%, 85%, 90%, 95%, 96%, 97%, 98%, 99%, or 100% sequence identity to SEQ ID NO: 59.

In some embodiments, the linker sequence can be cleavable linker. The cleavable linker can be cleaved by proteases, such as a metalloprotease. In some embodiments, the linker sequence is encoded by a polynucleotide sequence having at least 75%, 80%, 85%, 90%, 95%, 96%, 97%, 98%, 99%, or 100% sequence identity to SEQ ID NO: 60.

In some embodiments, the protease is selected from the group consisting of metalloproteases, Serine proteases, Cysteine proteases, threonine proteases, Aspartic proteases, Glutamic proteases and Asparagine proteases.

The linker sequence can be a monomer, thereby the linker can comprise at least 1, 2, 3, 4, or 5 monomers. In some embodiments, the linker can be a n-mer of cleavable linkers, non-cleavable linkers, or any combination thereof.

### CRISPR-Cas9

In some embodiments, the insertion is carried out using one or more DNA-binding nucleic acids, such as disruption via a nucleic acid-guided nuclease. For example, in some embodiments, the insertion is carried out using clustered regularly interspaced short palindromic repeats (CRISPR) and CRISPR-associated (Cas) proteins via introduction of a double-stranded break in a DNA sequence.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide polynucleotide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes or Staphylococcus aureus.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by disruptions as discussed herein. In other embodiments, Cas9 variants, deemed “nickases” are used to nick a single strand at the target site. In some embodiments, paired nickases are used, e.g., to improve specificity, each directed by a pair of different gRNAs targeting sequences such that upon introduction of the nicks simultaneously, a 5′ overhang is introduced.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “donor template” or “donor polynucleotide” or “donor sequence”. In some embodiments, an exogenous polynucleotide may be referred to as an donor template or donor polynucleotide. In some embodiments, the donor polynucleotide comprises an exogenous polynucleotide sequence. In some embodiments, the recombination is homologous recombination or homology-directed repair (HDR).

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, the nucleic acid guide programmable nuclease can be a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes, S. aureus or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme. Non-limiting examples of mutations in a Cas9 protein are known in the art (see e.g. WO2015/161276), any of which can be included in a CRISPR/Cas9 system in accord with the provided methods. In some embodiments, the CRISPR enzyme is mutated such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g. 1, 2, 3, 4, 5, 10, 15, 20, 25, 50, or more, or all codons) in a sequence encoding the CRISPR enzyme corresponds to the most frequently used codon for a particular amino acid.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, ClustalX, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide polynucleotide sequence reactions.

A guide polynucleotide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as may alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CR ISPR enzyme are described in US20110059502, incorporated herein by reference. In some embodiments, a tagged CRISPR enzyme is used to identify the location of a target sequence.

In some embodiments, a CRISPR enzyme in combination with (and optionally complexed with) a guide polynucleotide sequence is delivered to the cell. In some embodiments, methods for introducing a protein component into a cell according to the present disclosure (e.g. Cas9/gRNA RNPs) may be via physical delivery methods (e.g. electroporation, particle gun, Calcium Phosphate transfection, cell compression or squeezing), liposomes or nanoparticles.

For example, CRISPR/Cas9 technology may be used to knock-down gene expression of the target antigen in the engineered cells. In an exemplary method, Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target antigen gene are introduced into cells, for example, using lentiviral delivery vectors or any of a number of known delivery method or vehicle for transfer to cells, such as any of a number of known methods or vehicles for delivering Cas9 molecules and guide RNAs. Non-specific or empty vector control T cells also are generated. Degree of Knockout of a gene (e.g., 24 to 72 hours after transfer) is assessed using any of a number of well-known assays for assessing gene disruption in cells.

It is within the level of a skilled artisan to design or identify a gRNA sequence that is or comprises a sequence targeting a target antigen of interest, such as any described herein, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; http://www.e-crisp.org/E-CRISP/; http://crispr.mit.edu/; https://www.dna20.com/eCommerce/cas9/input). In some embodiments, the gRNA sequence is or comprises a sequence with minimal off-target binding to a non-target gene.

In some embodiments, design gRNA guide sequences and/or vectors for any of the antigens as described herein are generated using any of a number of known methods, such as those for use in gene knockdown via CRISPR-mediated, TALEN-mediated and/or related methods.

In some embodiments, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

Binding of the polynucleotide sequence recruits the Cas9 protein and facilitates a double-stranded break into the polynucleotide sequence by the Cas9 nuclease. In some embodiments, guide polynucleotide sequence binds to a region of a gene corresponding to the coding sequence. In some embodiments, the coding sequence is an exon. In some embodiments, the guide polynucleotide can bind to a region of the gene corresponding to a non-coding region. In some embodiments, the non-coding region is an intron or untranslated region (UTR).

Guide polynucleotide sequences are specific to the target that they bind. In some embodiments, the guide polynucleotide sequence target is a region of hemoglobin A (HBA1) or CCR5. In some embodiments, the guide polynucleotide sequence comprises at least 75% sequence identity to SEQ ID NO: 50 or SEQ ID NO: 51, or the reverse complement thereof. In some embodiments, the guide polynucleotide sequence comprises SEQ ID NO: 50 or SEQ ID NO: 51, or the reverse complement thereof. In some embodiments, the guide polynucleotide sequence binds to at least a portion of SEQ ID NO: 50 or SEQ ID NO: 51, or the reverse complement thereof.

In some embodiments, guide polynucleotide sequence comprises a chemical modification. In some embodiments, the guide polynucleotide sequence comprises a 2′-O-methyl-3′-phosphorothioate modification. Examples of chemical modifications to guide polynucleotide sequences which enhance stability and cleavage efficiency of CRISPR-Cas systems include but are not limited to those described in PCT Publication Nos. WO/2016164356 and WO 2016/089433, each of which is herein incorporated by reference in its entirety.

### Delivery Vectors

Provided herein are delivery vectors that will enable introduction of the compositions described herein into a cell. The delivery vector may include a surface modification that targets the vector to a cell of the subject, such as an antibody linked to an external surface of the viral delivery vector, wherein the antibody targets hematopoietic stem cells, or precursors thereof. The composition may include a particle (e.g., lipid nanoparticle or liposome) containing the globin gene and the gene editing reagents, or a plurality of lipid nanoparticles having the globin gene and the gene editing reagents comprised or embedded therein. For example, the plurality of lipid nanoparticles may include at least: a first solid lipid nanoparticle comprising a segment of DNA that includes the globin gene; a second solid lipid nanoparticle that includes at least one Cas endonuclease complexed with a guide RNA (gRNA) that targets the Cas endonuclease to a locus within an alpha-globin gene cluster in chromosome 16. The particle(s) may be provided as one or a plurality of liposomes enveloping one or more of the globin gene and the gene editing reagents.

Donor polynucleotide sequences described herein may be incorporated within a wide variety of gene therapy constructs, e.g., to deliver a nucleic acid encoding a protein to a subject in need thereof. A vector construct refers to a polynucleotide molecule including all or a portion of a viral genome and an exogenous polynucleotide sequence. In some instances, gene transfer can be mediated by a DNA viral vector, such as an adenovirus (Ad) or adeno-associated virus (AAV). Other vectors useful in methods of gene therapy are known in the art. For example, a construct of the present invention can include analphavirus, herpesvirus, retrovirus, lentivirus, or vaccinia virus.

Adenoviruses are a relatively well characterized group of viruses, including over 50 serotypes. Adenoviruses are tractable through the application of techniques of molecular biology and may not require integration into the host cell genome. Recombinant Ad-derived vectors, including vectors that reduce the potential for recombination and generation of wild-type virus, have been constructed. Wild-type AAV has high infectivity and is capable of integrating into a host genome with a high degree of specificity.

AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally.

At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure.

A serotype of a viral vector used in certain embodiments of the invention can be selected from the group consisting from AAV1, AAV2, AAV3, AAV4, AAV5, AAV6, AAV7, AAV8, and AAV9. Other serotypes are known in the art or described herein and are also applicable to the present disclosure. In particular instances, the present invention includes an AAV9 viral vector including a glucocerebrosidase nucleic acid of the present invention.

A vector of the present invention can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV).

Without limitation, illustrative examples of pseudotyped vectors include recombinant AAV2/1, AAV2/2, AAV2/5, AAV2/6, AAV2/7, and AAV2/8 serotype vectors. It is known in the art that such vectors may be engineered to include a transgene encoding a human protein or other protein. In particular instances, the present invention includes a AAV6 vector for delivery.

In some instances, a particular AAV serotype vector may be selected based upon the intended use, e.g., based upon the intended route of administration. For example, for direct injection into the brain, e.g., either into the striatum, an AAV2 serotype vector can be used.

Various methods for application of AAV vector constructs in gene therapy are known in the art, including methods of modification, purification, and preparation for administration to human.

### Methods of Use

Provided herein are methods of treatment for diseases and disorders using the composition of the present disclosure. The present disclosure provides composition to genetically modify cells, such as HSPCs, to generate modified HSPCs that express a therapeutic protein linked to a transmembrane domain. The present disclosure provides non-limiting examples of diseases and disorders that are amenable to the use of the composition of this disclosure to treat said diseases or disorders. The diseases or disorders can be, but is not limited to, hereditary angioedema (HAE), Hemophilia A, Hemophilia B, Phenylketonuria (PKU), or any other genetic disease in which the presence of a circulating protein can provide therapeutic benefit to said diseases or disorders.

In another embodiment, the present disclosure can provide methods that can be used in the production of antibodies.

### Alpha Antitrypsin Deficiency

α1-antitrypsin deficiency (AATD) is a genetic disorder characterized by a predisposition for the development of a number of diseases, mainly pulmonary emphysema and other chronic respiratory disorders with different clinical manifestations and frequent overlap, and several types of hepatopathies in both children and adults.

AAT is the most prevalent proteases inhibitor in the human serum. It is primarily produced in high quantities and secreted mainly by hepatocytes. AAT is an important anti-protease in the lung, but it also has significant anti-inflammatory effects on several cell types and modulates inflammation caused by host and microbial factors. It can play an important role in modulating key immune cell activities and protecting the lungs against damage caused by proteases and inflammation.

The present disclosure provides methods and compositions to treat alpha-antitrypsin deficiency.

Treatment using the compositions and methods of the present disclosure is introduced into a cell. In some embodiments, the cell is obtained from a subject in need of treatment. Cells are contacted with the composition described herein to generate a genetically modified cell with an altered expression profile. The genetically modified cell is re-introduced into the subject to treat the disease or disorder thereof.

In some embodiments, the subject is human. In some embodiments, the cell is a primary cell. In some embodiments, the cell is a CD34+ cell. In some embodiments, the cell is a hematopoietic stem or progenitor cell. In some embodiments, the cells are obtained from an apheresis product obtained from the donor or subject. In some embodiments, the subject is human.

### Genetically Modified Cells

Provided herein is a genetically modified cell, wherein the genetically modified cell is prepared according to the method disclosed herein. In some embodiments, the genetically modified cells are prepared by introducing into a cell the programmable nucleic acid-guided nuclease and guide polynucleotide sequence. In addition, the donor polynucleotide sequence can be administered. Through a single recombination event, at least a portion of the donor polynucleotide sequence is integrated into a region of the target site of the cell. After targeted gene integration through resolution of a single recombination event between the donor polynucleotide and the endogenous target site, expression of the target gene can be different compared to a cell that has not been genetically modified using the method disclosed in the present disclosure.

In some embodiments, the genetically modified cell has greater expression of a gene following targeted gene insertion compared to a cell that has not been genetically modified. In some embodiments, the genetically modified cell comprises about 50% greater expression to about 100% greater expression compared to a cell that has not been genetically modified. In some embodiments, the genetically modified cell comprises at least about 50% greater expression. In some embodiments, the genetically modified cell comprises at most about 100% greater expression. In some embodiments, the genetically modified cell comprises about 50% greater expression to about 60% greater expression, about 50% greater expression to about 70% greater expression, about 50% greater expression to about 80% greater expression, about 50% greater expression to about 90% greater expression, about 50% greater expression to about 100% greater expression, about 60% greater expression to about 70% greater expression, about 60% greater expression to about 80% greater expression, about 60% greater expression to about 90% greater expression, about 60% greater expression to about 100% greater expression, about 70% greater expression to about 80% greater expression, about 70% greater expression to about 90% greater expression, about 70% greater expression to about 100% greater expression, about 80% greater expression to about 90% greater expression, about 80% greater expression to about 100% greater expression, or about 90% greater expression to about 100% greater expression compared to a cell that has not been genetically modified.

In some embodiments, the genetically modified cell is prepared or generated ex vivo.

In some embodiments, the genetically modified cell is obtained from a subject, for example, a subject in need of the therapeutic protein introduced by the genetic modification.

In some embodiments, the cell to be genetically modified is a primary cell. In some embodiments, the primary cell is a mammalian primary cell. In some embodiments, the primary cell is a human cell. In some embodiments, the primary cell is selected from the group consisting of a primary blood cell and a primary mesenchymal cell. In some embodiments, the primary cell is selected from the group consisting of a primary stem cell, primary progenitor cell, and primary somatic cell. In some embodiments, the stem cell selected from the group consisting of an embryonic stem cell, induced pluripotent stem cell, hematopoietic stem cell, mesenchymal stem cell, neural stem cell, and organ stem cell. In some embodiments, the progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell. In some embodiments, the somatic cell is selected from the group consisting of a fibroblast, a hepatocyte, a heart cell, a liver cell, a pancreatic cell, a muscle cell, a skin cell, a blood cell, a neural cell, and an immune cell. In some embodiments, the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell. In some embodiments, the primary cell is a CD34+ hematopoietic stem and progenitor cell (HSPC).

HSPCs can be modified by introduction of an engineered guide polynucleotide specific for a target gene. A donor polynucleotide comprising a polynucleotide sequence encoding a therapeutic protein is also introduced in order to provide targeted stable integration of the donor polynucleotide into the cell. The process produces an engineered cell or engineered HSPC; or genetically modified cell or genetically modified HSPC.

In some embodiments, it may be desirable to expand and partially differentiate the HSPC (or CD34+ HSPC) in vitro and to allow terminal differentiation into mature erythrocytes to occur in vivo or in vitro (See, e.g., Neildez-Nguyen et al., Nature Biotech. 20:467-472 (2002)). Isolated CD34+ hematopoietic stem cells may be expanded in vitro in the absence of the adherent stromal cell layer in medium containing various factors including, for example, Flt3 ligand, stem cell factor, thrombopoietin, erythropoietin, and insulin growth factor. The resulting erythroid precursor cells may be characterized by the surface expression of CD36 and GPA and may be transfused into a subject where terminal differentiation to mature erythrocytes is allowed to occur. Such cells would still retain expression of the exogenous polynucleotide such that the erythrocyte would be covered with the therapeutic protein of the present disclosure.

The genetically modified cell can express a therapeutic protein on its cell surface, wherein the therapeutic protein is tethered to the cell surface by a linker to a transmembrane domain. In some embodiments, the therapeutic protein is expressed in its active form. The therapeutic protein can be linked by a cleavable linker, and the linker can be cleaved by a specific protease, thereby releasing the active therapeutic protein into circulation.

In some embodiments, the therapeutic protein is expressed in its inactive form. The therapeutic protein can be linked by a cleavable linker, and the linker can be cleaved by a specific protease, thereby releasing the inactive therapeutic protein into circulation. In some embodiments, upon cleavage of the cleavable linker, the inactive therapeutic protein becomes active.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals. In some embodiments, the modified cells of the pharmaceutical composition are autologous to the individual in need thereof. In other embodiments, the modified cells of the pharmaceutical composition are allogeneic to the individual in need thereof.

In some embodiments, a pharmaceutical composition comprising a modified host cell as described herein is provided. In some embodiments, the modified host cell is genetically engineered to comprise an integrated donor sequence, including, for example, coding sequences for a gene of interest and optionally other regulatory sequences, at a targeted gene locus of the host cell. In some embodiments, a therapeutic donor sequence is integrated into the translational start site of the endogenous gene locus. In some embodiments, the therapeutic donor sequence that is integrated into the host cell genome is expressed under control of the native promoter sequence of the targeted gene locus of the host cell. In some embodiments, the modified host cell is genetically engineered to comprise an integrated therapeutic donor sequence, including, for example, coding sequences for a therapeutic protein operably linked to a transmembrane domain via a linker, at a safe harbor locus such as HBA1 or CCR5. In particular embodiments, a therapeutic donor sequence is integrated into the translational start site of the endogenous safe harbor locus. In particular embodiments, the therapeutic donor sequence that is integrated into the host cell genome is expressed under control of the native promoter sequence of the safe harbor locus.

In some embodiments, the pharmaceutical composition comprises a plurality of the modified host cells, and further comprises unmodified host cells and/or host cells that have undergone nuclease cleavage resulting in INDELS at the safe harbor locus but not integration of the therapeutic donor sequence. In some embodiments, the pharmaceutical composition is comprised of at least 5% of the modified host cells comprising an integrated therapeutic donor sequence. In some embodiments, the pharmaceutical composition is comprised of about 9% to 50% of the modified host cells comprising an integrated therapeutic donor sequence. In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated therapeutic donor sequence. The pharmaceutical compositions described herein may be formulated using one or more excipients to, e.g.: (1) increase stability; (2) alter the biodistribution (e.g., target the cells to specific tissues or cell types, e.g. HSPCs); and/or (3) enhance engraftment in the recipient.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g. the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regimen before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing a hemoglobinopathy or other disease described herein. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years. In some embodiments, only a single dose is needed to effect treatment or prevention of a disease or disorder described herein. In other embodiments, a subject in need thereof may receive more than one dose, for example, 2, 3, or more than 3 doses of a modified host cell pharmaceutical compositions described herein to effect treatment or prevention of the disease or disorder.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of a hemoglobinopathy or other disease described herein is also encompassed by the disclosure.

The present disclosure also contemplates kits comprising compositions or components of the present disclosure, e.g., sgRNA, Cas nuclease, RNPs, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Design of Protein Display Construct

To test whether a therapeutic protein can be introduced into a cell and expressed in accordance with the methods described herein, multiple donor polynucleotides were designed in order to assess which donor polynucleotide is most effective in integrating into a cell and expressing a therapeutic protein on its surface.

The tested constructs are summarized in Table 1 and Table 2.

### HBA1 Targeting Constructs

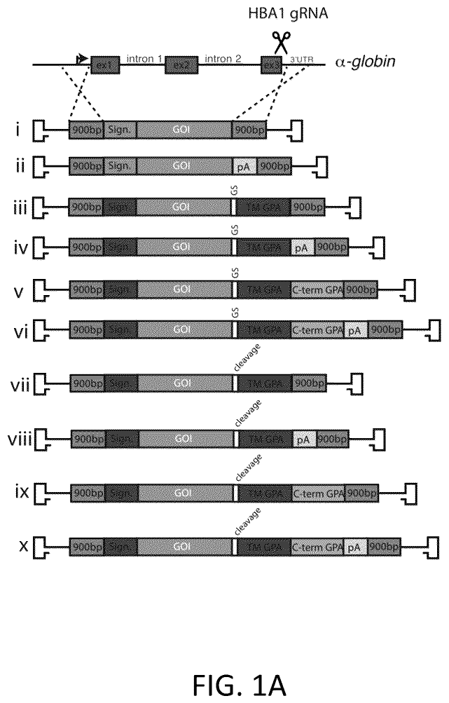

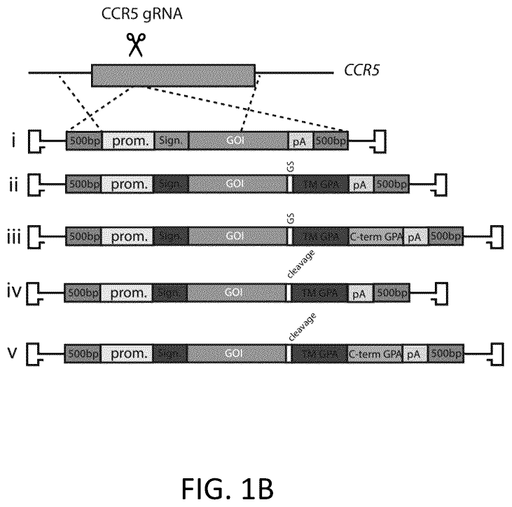

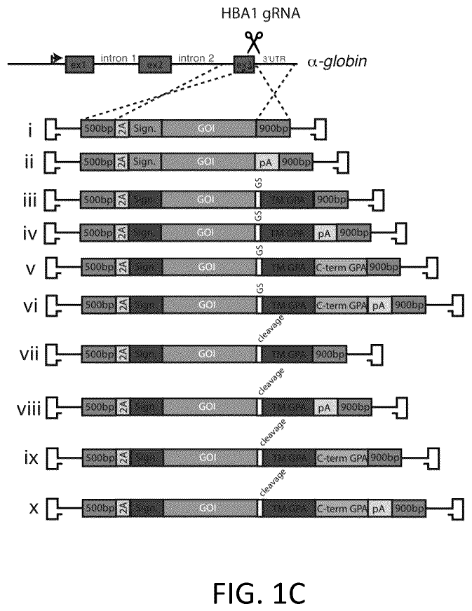

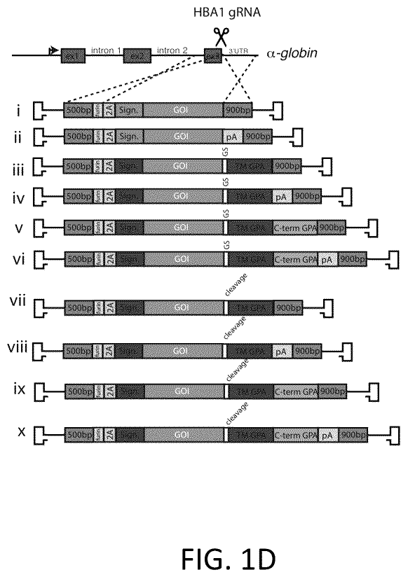

To assess HBA1 targeting, FIG. 1A (i-x) shows a 900 bp 5′ and 900 bp 3′ homology arm flanking either end of the exogenous polynucleotide which allows for targeted integration via homology directed repair and replaces the entirety of the coding sequence of HBA1, as denoted by the dotted lines. The exogenous polynucleotide can include different signal peptides as shown in FIG. 1A (i-ii) and FIG. 1A(iii-x). In FIG. 1A (iii-v) a non-cleavable linker is used as denoted by “GS,” whereas a cleavable linker was used in the constructs of FIG. 1A (vii-x). In FIG. 1A (iii-x), a transmembrane domain was used, where in FIG. 1A (v, vi, ix, x) a C-terminal tail of GPA was appended to the end of the GPA transmembrane domain. In some instances, a polyadenylation site is added.

To assess HBA1 targeting that replaces exon 3, FIG. 1C (i-x) shows similar constructs to FIG. 1A (i-x), except a 5′ 500 bp and 3′ 900 bp homology arm flanked the exogenous polynucleotide. To eliminate the generation of the fusion protein encoded by the exogenous polynucleotide being linked to HBA1, a sequence encoding the self-cleaving 2A peptide was added to the 5′ end of the exogenous polynucleotide. In FIG. 1D (i-x), a sequence encoding a furin cleavage site was added to the 5′ end of the 2A peptide.

### CCR5 Targeting Constructs

To assess CCR5 targeting constructs, similar exogenous polynucleotides as FIG. 1A were designed but used a 500 bp 5′ and 500 bp 3′ homology arm to direct CCR5 integration by homology directed repair. In addition, a red-blood cell specific promoter was inserted at the 5′ end of the exogenous polynucleotides, instead of using the endogenous CCR5 promoter to promote better expression.

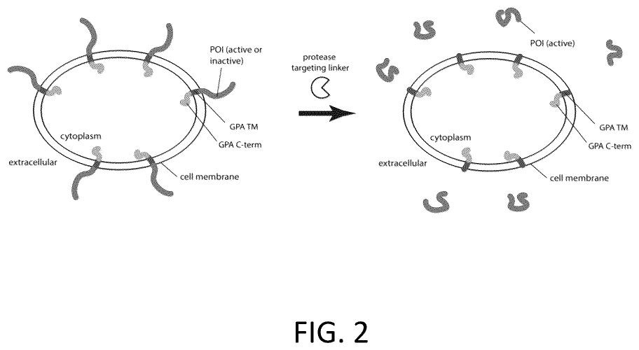

FIG. 2 shows a schematic of an embodiment of the disclosure, wherein the cell is decorated with the protein of interest (POI) and can be cleaved off upon addition of the protease that targets the specific cleavable linker.

### Example 2: Expression of the Therapeutic Fusion Protein on Cells

### Cell Culture

HEK293T cells were cultured in DMEM (Gibco) supplemented with 10% Fetal Bovine Serum (FBS, Gibco) and 1% penicillin-streptomycin (Gibco). HEK293T cells were passaged every 2-3 days. Cells were grown in a humidified 37° C. incubator with 5% CO2.

### Transient Transfections of HEK293T Cells

HEK293T cells (1×10⁶) were seeded in each well of a 6-well plate a day before transfection to reach a confluency between 80-90% at the day of transfection. HEK293T cells were transfected using TransIT-LT1Transfection Reagent (Mirus Bio) according to the manufacturer's instructions. Briefly, a 250 μL of Opti-MEM I Reduced-Serum Medium (Gibco), 2.5 μg plasmid DNA and 7.5 μL TranslT-LT1 Reagent was mixed and then added to the cells. Cells were then cultured for 48-72 hours before cell harvest.

### Western Blot

Cells were harvested by lifting them off the culture plate and then washed with Phosphate buffer saline (PBS). Cell pellets were lysed on ice for 30 mins with Cell Lysis Buffer (Invitrogen) supplemented with 1× Halt™ Protease Inhibitor Cocktail (Thermo Scientific). Lysate concentrations were quantified by a DS-11 series Spectrophotometer/Fluorometer (DeNovix). Samples for SDS Page were prepared in 4× Laemmli Sample Buffer (Bio-Rad) supplemented with 10% 2-Mercaptoethanol (Fisher BioReagents) and run on a 4-15% Mini-PROTEAN TGX Stain-Free Protein Gel (Bio-Rad) in 1× Tris/Glycine/SDS Buffer (Bio-Rad). Proteins were transferred to a membrane using a Trans-Blot Turbo Mini 0.2 μm Nitrocellulose Transfer Pack (Bio-Rad) and a Trans-Blot Turbo transfer System (Bio-Rad). Membranes were blocked in 5% nonfat dairy milk in 1× Tris-buffered Saline with 0.1% Tween-20 (TBS-T) overnight at 40 C. The blots were incubated in primary antibody diluted in TBS-T:Blocking Buffer (Rockland Immunochemicals) (1:1) for 2 hours. Blots were probed with antibodies against alpha-1 Antitrypsin (Invitrogen, PA5-16661) and myc-Tag (9B11)(Cell Signaling Technology, 2276). Blots were developed after incubation with Starbright Blue 520 Goat anti-Mouse IgG (Bio-Rad) and Starbright Blue 700 Goat anti-Rabbit IgG (Bio-Rad) in TBS-T:Blocking Buffer(Rockland Immunochemicals) (1:1) for 1 hour. Blots were imaged using a ChemiDoc MP Imaging System (Bio-Rad).

### Flow Cytometry

The cells were analyzed for expression of the myc epitope tag and AAT using a Cytoflex flow cytometer (Beckman Coulter). For cell surface staining, cells were pelleted and resuspended in PBS supplemented with 0.5% BSA containing antibodies against myc-tag (9B11, Alexa Fluor 647 Conjugate) (Cell Signalling Technology, 2233) and alpha-1 Antitrypsin (Invitrogen, PA5-16661). Cells were incubated with staining solution at room temperature for 30 minutes. Cells were pelleted again and resuspended in PBS supplemented with 0.5% BSA containing Starbright Blue 700 Goat anti-Rabbit IgG secondary antibody (Bio-Rad, 12004161). Cells were incubated with staining solution at room temperature and covered by foil for 30 minutes. Cells were washed with PBS (with 0.5% BSA). Cells were then resuspended in PBS supplemented with 0.5% BSA containing live/dead cell stain (DAPI staining solution, Miltenyi Biotec) and subjected to flow cytometry. Analysis was performed using FlowJo software. During analysis, cells were gated for single cells, live cells, myc+ and AAT+.

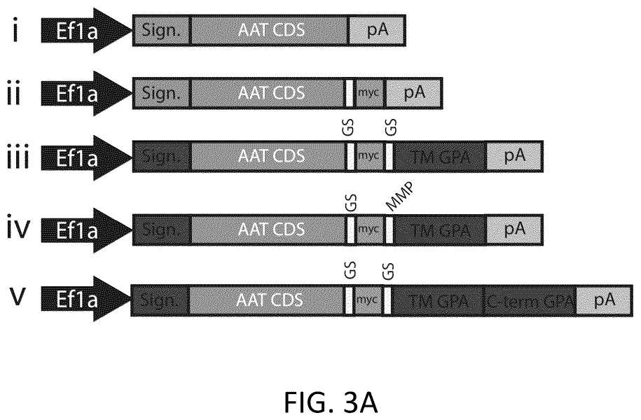

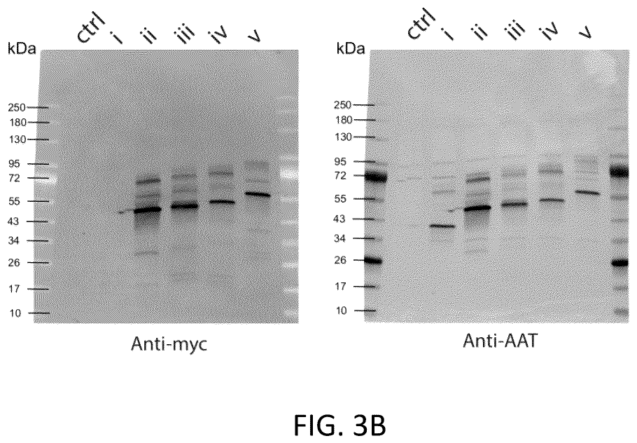

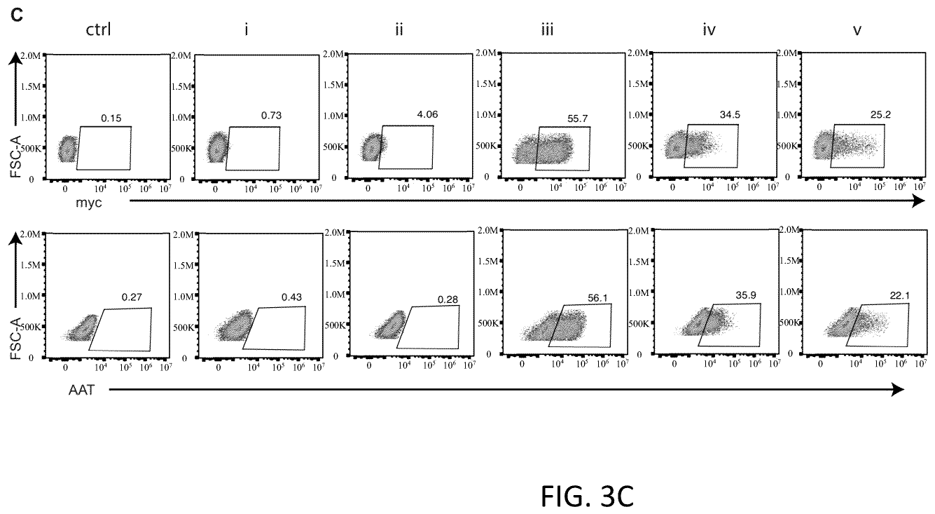

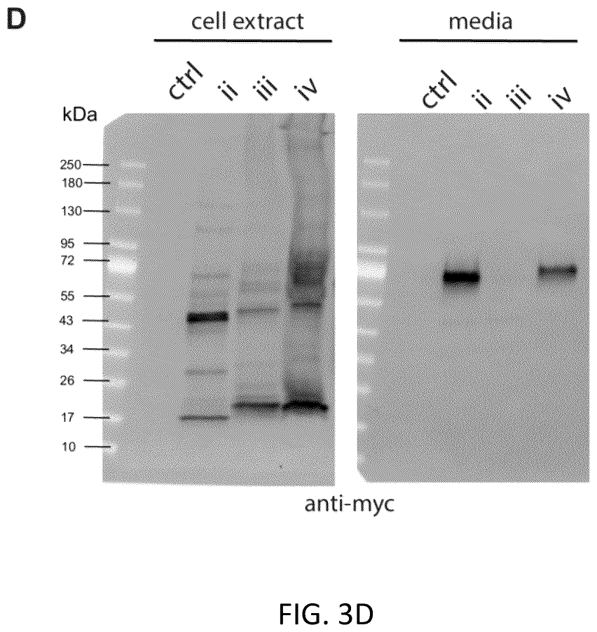

To assess whether cells can express a therapeutic protein on its cell surface, transient transfection of alpha-antitrypsin linked to a GPA transmembrane domain was introduced to HEK 293T cells and its expression was measured. The different expression constructs are shown in FIG. 3A. FIG. 3B provides a western blot of cell lysates probed with either an anti-myc antibody or an anti-AAT antibody, which shows that the AAT protein is expressed by the cell.

By flow cytometry, AAT expression was assessed by staining cells with an anti-AAT antibody under non-permeabilizing conditions, such that only surface expressed AAT is detectable via flow cytometry. In FIG. 3C, constructs iii-v show increased signal in the AAT channel, suggesting that AAT is on the surface of cells, but is not on the surface when no transmembrane domain is present.

To assess if AAT is released into the media, the cell extract or the media from transfected cells were probed by an anti-myc antibody via Western blot. As shown in FIG. 3D, constructs ii and iv show release of the AAT protein into the media.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
Polynucleotide Sequences

| SEQ ID | | |
|---|---|---|
| NO: | Name | Sequence |
| 1 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | 3′ homology | gtgcctgtcactcaagcacactagtgactatcgccagagggaaagggagctgcaggaagcgaggctggaga |
| | arm | gcaggaggggctctgcgcagaaattcttttgagttcctatgggccagggcgtccgggtgcgcgcattcctc |
| | | tccgccccaggattgggcgaagcctcccggctcgcactcgctcgcccgtgtgttccccgatcccgctggag |
| | | tcgatgcgcgtccagcgcgtgccaggccggggcgggggtgcgggctgactttctccctcgctagggacgct |
| | | ccggcgcccgaaaggaaagggtggcgctgcgctccggggtgcacgagccgacagcgcccgaccccaacggg |
| | | ccggccccgccagcgccgctaccgccctgcccccgggcgagcgggatggggggagtggagtggcgggtgga |
| | | gggtggagacgtcctggcccccgccccgcgtgcacccccaggggaggccgagcccgccgcccggccccgcg |
| | | caggccccgcccgggactcccctgcggtccaggccgcgccccgggctccgcgccagccaatgagcgccgcc |
| | | cggccgggcgtgcccccgcgccccaagcataaaccctggcgcgctcgcggcccggcactcttctggtcccc |
| | | acagactcagagagaacccaccATGCCTTCATCAGTATCTTGGGGAATACTGCTCCTTGCTGGGTTGTGTT |
| | | GTCTCGTACCCGTGAGTCTCGCCGAAGACCCTCAAGGCGACGCCGCACAAAAGACTGACACTTCTCATCAC |
| | | GACCAAGACCATCCTACATTTAATAAAATTACTCCAAATCTCGCCGAATTTGCGTTTTCTCTGTATAGGCA |
| | | ACTCGCTCACCAATCTAATTCAACGAACATATTCTTTTCACCTGTTTCCATAGCCACCGCTTTCGCCATGC |
| | | TGAGTTTGGGAACAAAAGCAGATACCCATGACGAGATACTCGAAGGACTCAACTTTAATCTGACAGAAATC |
| | | CCTGAAGCACAAATTCACGAGGGTTTTCAAGAGCTGCTGAGAACTTTGAATCAACCCGATTCCCAATTGCA |
| | | ACTCACAACAGGAAACGGTTTGTTTCTTTCAGAAGGGCTCAAACTGGTCGACAAATTCCTCGAAGACGTGA |
| | | AGAAACTTTATCATAGCGAGGCTTTTACCGTGAATTTTGGAGATACGGAAGAAGCTAAGAAGCAAATAAAT |
| | | GACTATGTCGAAAAGGGGACACAGGGAAAGATAGTTGACCTGGTGAAAGAACTGGATAGGGATACTGTGTT |
| | | CGCGCTCGTCAACTATATCTTCTTCAAGGGGAAGTGGGAACGGCCATTCGAGGTTAAAGATACAGAAGAGG |
| | | AAGATTTTCATGTAGATCAAGTCACAACAGTCAAAGTTCCAATGATGAAACGCCTCGGGATGTTCAATATA |
| | | CAACATTGCAAGAAACTTAGCTCATGGGTCCTTTTGATGAAGTATCTCGGGAACGCTACAGCGATATTCTT |
| | | TCTCCCAGACGAAGGTAAGCTGCAACATCTTGAGAACGAGCTGACACATGACATAATAACAAAATTTCTTG |
| | | AGAACGAGGATCGCCGGTCCGCATCCCTGCACCTGCCGAAGCTTAGCATAACCGGCACATACGACTTGAAA |
| | | TCTGTTCTTGGGCAGCTTGGTATTACAAAAGTGTTTTCCAACGGCGCGGATCTGTCAGGCGTGACGGAAGA |
| | | AGCTCCTCTTAAACTGAGTAAAGCAGTCCACAAAGCAGTACTCACTATTGATGAAAAGGGTACCGAGGCGG |
| | | CCGGAGCTATGTTCCTCGAAGCTATTCCTATGAGTATTCCCCCTGAAGTTAAATTTAATAAGCCTTTCGTG |
| | | TTTCTCATGATAGAGCAGAACACGAAAAGCCCTCTGTTTATGGGCAAGGTCGTCAACCCAACACAGAAGTA |
| | | Gtggccatgcttcttgccccttgggcctccccccagcccctcctccccttcctgcacccgtacccccgtgg |
| | | tctttgaataaagtctgagtgggggcagcctgtgtgtgcctgagttttttccctcagcaaacgtgccaggc |
| | | atgggcgtggacagcagctgggacacacatggctagaacctctctgcagctggatagggtaggaaaaggca |
| | | ggggcgggaggaggggatggaggagggaaagtggagccaccgcgaagtccagctggaaaaacgctggaccc |
| | | tagagtgctttgaggatgcatttgctctttcccgagttttattcccagacttttcagattcaatgcaggtt |
| | | tgctgaaataatgaatttatccatctttacgtttctgggcactctgtgccaagaactggctggctttctgc |
| | | ctgggacgtcactggtttcccagaggtcctcccacatatgggtggtgggtaggtcagagaagtcccactcc |
| | | agcatggctgcattgatcccccatcgttcccactagtctccgtaaaacctcccagatacaggcacagtcta |
| | | gatgaaatcaggggtgcggggtgcaactgcaggccccaggcaattcaataggggctctactttcaccccca |
| | | ggtcaccccagaatgctcacacaccagacactgacgccctggggctgtcaagatcaggcgtttgtctctgg |
| | | gcccagctcagggcccagctcagcacccactcagctcccctgaggctggggagcctgtcccattgcgactg |
| | | gagaggagagcggggccacagaggcctggctagaaggtcccttctccctggtgtgtgttttctctctgctg |
| | | agcaggcttgcagtgcctggggtatca |
| 2 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein-pA- | aggagtctgaatctggagccgcccccagcccagccccgtgctttttgcgtcctggtgtttattccttcccg |
| | 3′ homology | gtgcctgtcactcaagcacactagtgactatcgccagagggaaagggagctgcaggaagcgaggctggaga |
| | arm | gcaggaggggctctgcgcagaaattcttttgagttcctatgggccagggcgtccgggtgcgcgcattcctc |
| | | tccgccccaggattgggcgaagcctcccggctcgcactcgctcgcccgtgtgttccccgatcccgctggag |
| | | tcgatgcgcgtccagcgcgtgccaggccggggcgggggtgcgggctgactttctccctcgctagggacgct |
| | | ccggcgcccgaaaggaaagggtggcgctgcgctccggggtgcacgagccgacagcgcccgaccccaacggg |
| | | ccggccccgccagcgccgctaccgccctgcccccgggcgagcgggatgggcgggagtggagtggcggggga |
| | | gggtggagacgtcctggcccccgccccgcgtgcacccccaggggaggccgagcccgccgcccggccccgcg |
| | | caggccccgcccgggactcccctgcggtccaggccgcgccccgggctccgcgccagccaatgagcgccgcc |
| | | cggccgggcgtgcccccgcgccccaagcataaaccctggcgcgctcgcggcccggcactcttctggtcccc |
| | | acagactcagagagaacccaccATGCCTTCATCAGTATCTTGGGGAATACTGCTCCTTGCTGGGTTGTGTT |
| | | GTCTCGTACCCGTGAGTCTCGCCGAAGACCCTCAAGGCGACGCCGCACAAAAGACTGACACTTCTCATCAC |
| | | GACCAAGACCATCCTACATTTAATAAAATTACTCCAAATCTCGCCGAATTTGCGTTTTCTCTGTATAGGCA |
| | | ACTCGCTCACCAATCTAATTCAACGAACATATTCTTTTCACCTGTTTCCATAGCCACCGCTTTCGCCATGC |
| | | TGAGTTTGGGAACAAAAGCAGATACCCATGACGAGATACTCGAAGGACTCAACTTTAATCTGACAGAAATC |
| | | CCTGAAGCACAAATTCACGAGGGTTTTCAAGAGCTGCTGAGAACTTTGAATCAACCCGATTCCCAATTGCA |
| | | ACTCACAACAGGAAACGGTTTGTTTCTTTCAGAAGGGCTCAAACTGGTCGACAAATTCCTCGAAGACGTGA |
| | | AGAAACTTTATCATAGCGAGGCTTTTACCGTGAATTTTGGAGATACGGAAGAAGCTAAGAAGCAAATAAAT |
| | | GACTATGTCGAAAAGGGGACACAGGGAAAGATAGTTGACCTGGTGAAAGAACTGGATAGGGATACTGTGTT |
| | | CGCGCTCGTCAACTATATCTTCTTCAAGGGGAAGTGGGAACGGCCATTCGAGGTTAAAGATACAGAAGAGG |
| | | AAGATTTTCATGTAGATCAAGTCACAACAGTCAAAGTTCCAATGATGAAACGCCTCGGGATGTTCAATATA |
| | | CAACATTGCAAGAAACTTAGCTCATGGGTCCTTTTGATGAAGTATCTCGGGAACGCTACAGCGATATTCTT |
| | | TCTCCCAGACGAAGGTAAGCTGCAACATCTTGAGAACGAGCTGACACATGACATAATAACAAAATTTCTTG |
| | | AGAACGAGGATCGCCGGTCCGCATCCCTGCACCTGCCGAAGCTTAGCATAACCGGCACATACGACTTGAAA |
| | | TCTGTTCTTGGGCAGCTTGGTATTACAAAAGTGTTTTCCAACGGCGCGGATCTGTCAGGCGTGACGGAAGA |
| | | AGCTCCTCTTAAACTGAGTAAAGCAGTCCACAAAGCAGTACTCACTATTGATGAAAAGGGTACCGAGGCGG |
| | | CCGGAGCTATGTTCCTCGAAGCTATTCCTATGAGTATTCCCCCTGAAGTTAAATTTAATAAGCCTTTCGTG |
| | | TTTCTCATGATAGAGCAGAACACGAAAAGCCCTCTGTTTATGGGCAAGGTCGTCAACCCAACACAGAAGTA |
| | | GCTGTGCCTTCTAGTTGCCAGCCATCTGTTGTTTGCCCCTCCCCCGTGCCTTCCTTGACCCTGGAAGGTGC |
| | | CACTCCCACTGTCCTTTCCTAATAAAATGAGGAAATTGCATCGCATTGTCTGAGTAGGTGTCATTCTATTC |
| | | TGGGGGGTGGGGTGGGGCAGGACAGCAAGGGGGAGGATTGGGAAGACAATAGCAGGCATGCTGGGGAtggc |
| | | catgcttcttgccccttgggcctccccccagcccctcctccccttcctgcacccgtacccccgtggtcttt |
| | | gaataaagtctgagtgggcggcagcctgtgtgtgcctgagttttttccctcagcaaacgtgccaggcatgg |
| | | gcgtggacagcagctgggacacacatggctagaacctctctgcagctggatagggtaggaaaaggcagggg |
| | | cgggaggaggggatggaggagggaaagtggagccaccgcgaagtccagctggaaaaacgctggaccctaga |
| | | gtgctttgaggatgcatttgctctttcccgagttttattcccagacttttcagattcaatgcaggtttgct |
| | | gaaataatgaatttatccatctttacgtttctgggcactctgtgccaagaactggctggctttctgcctgg |
| | | gacgtcactggtttcccagaggtcctcccacatatgggtggtgggtaggtcagagaagtcccactccagca |
| | | tggctgcattgatcccccatcgttcccactagtctccgtaaaacctcccagatacaggcacagtctagatg |
| | | aaatcaggggtgcggggtgcaactgcaggccccaggcaattcaataggggctctactttcacccccaggtc |
| | | accccagaatgctcacacaccagacactgacgccctggggctgtcaagatcaggcgtttgtctctgggccc |
| | | agctcagggcccagctcagcacccactcagctcccctgaggctggggagcctgtcccattgcgactggaga |
| | | ggagagcggggccacagaggcctggctagaaggtcccttctccctggtgtgtgttttctctctgctgagca |
| | | ggcttgcagtgcctggggtatca |
| 3 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | noncleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | 3′ homology | tccgccccaggattgggcgaagcctcccggctcgcactcgctcgcccgtgtgttccccgatcccgctggag |
| | arm | tcgatgcgcgtccagcgcgtgccaggccggggcgggggtgcgggctgactttctccctcgctagggacgct |
| | | ccggcgcccgaaaggaaagggtggcgctgcgctccggggtgcacgagccgacagcgcccgaccccaacggg |
| | | ccggccccgccagcgccgctaccgccctgcccccgggcgagcgggatggggggagtggagtggcgggtgga |
| | | gggtggagacgtcctggcccccgccccgcgtgcacccccaggggaggccgagcccgccgcccggccccgcg |
| | | caggccccgcccgggactcccctgcggtccaggccgcgccccgggctccgcgccagccaatgagcgccgcc |
| | | cggccgggcgtgcccccgcgccccaagcataaaccctggcgcgctcgcggcccggcactcttctggtcccc |
| | | acagactcagagagaacccaccATGTACGGGAAGATTATTTTCGTGTTGTTGCTCAGTGAGATCGTTTCTA |
| | | TCTCCGCTGAAGACCCTCAAGGCGACGCCGCTCAAAAGACGGACACGAGCCATCACGACCAAGACCATCCC |
| | | ACGTTTAATAAAATAACACCTAATCTCGCCGAATTTGCATTTTCACTGTATAGGCAACTCGCCCATCAAAG |
| | | CAATTCTACAAACATTTTCTTTAGCCCGGTCAGTATAGCGACTGCTTTCGCCATGCTGTCTCTCGGTACAA |
| | | AAGCCGATACCCATGACGAGATTTTGGAAGGACTCAACTTTAATCTGACCGAAATACCCGAAGCACAAATT |
| | | CACGAGGGGTTTCAAGAGCTGCTGCGAACTTTGAATCAACCCGATTCCCAACTGCAACTCACGACAGGTAA |
| | | CGGGTTGTTTCTGAGTGAAGGGCTTAAACTGGTTGACAAATTCCTGGAAGACGTGAAGAAACTCTATCATA |
| | | GTGAGGCATTTACAGTTAATTTTGGAGATACCGAGGAAGCTAAGAAGCAAATTAATGACTATGTTGAGAAA |
| | | GGCACGCAGGGAAAGATCGTTGACCTCGTGAAAGAACTGGATCGAGATACGGTGTTCGCCCTCGTCAACTA |
| | | TATATTCTTCAAGGGGAAGTGGGAACGCCCATTCGAGGTGAAAGATACAGAAGAGGAAGATTTTCATGTAG |
| | | ATCAAGTTACAACAGTAAAAGTACCCATGATGAAAAGACTCGGGATGTTCAATATCCAACATTGCAAGAAG |
| | | TTGTCATCTTGGGTCCTTTTGATGAAGTATCTTGGGAACGCAACGGCTATATTCTTTCTCCCGGACGAAGG |
| | | CAAGCTGCAACATCTCGAGAACGAGCTGACTCATGACATTATTACGAAATTTCTTGAGAACGAGGATCGCC |
| | | GGAGCGCGTCCCTGCACCTGCCTAAGCTCAGCATAACAGGTACGTACGACCTCAAATCCGTGTTGGGACAG |
| | | TTGGGGATTACGAAAGTGTTTTCCAACGGAGCGGATCTGAGCGGTGTGACCGAAGAAGCTCCACTTAAACT |
| | | GTCAAAAGCGGTCCACAAAGCCGTTTTGACTATAGATGAAAAGGGTACAGAGGCCGCCGGCGCGATGTTCC |
| | | TGGAAGCTATCCCGATGTCCATACCACCAGAAGTGAAATTTAATAAGCCTTTCGTGTTTCTGATGATAGAG |
| | | CAGAACACAAAATCCCCACTGTTTATGGGCAAGGTCGTCAACCCAACACAGAAGGGTTCAGGCGGGTCCGG |
| | | CGGAAGTGGGCTGATAATATTCGGCGTTATGGCCGGCGTGATCGGTACAATTCTGCTCATCAGTTATGGGA |
| | | TACGGAGATTGTGAtggccatgcttcttgccccttgggcctccccccagcccctcctccccttcctgcacc |
| | | cgtacccccgtggtctttgaataaagtctgagtgggcggcagcctgtgtgtgcctgagttttttccctcag |
| | | caaacgtgccaggcatgggcgtggacagcagctgggacacacatggctagaacctctctgcagctggatag |
| | | ggtaggaaaaggcaggggcgggaggaggggatggaggagggaaagtggagccaccgcgaagtccagctgga |
| | | aaaacgctggaccctagagtgctttgaggatgcatttgctctttcccgagttttattcccagacttttcag |
| | | attcaatgcaggtttgctgaaataatgaatttatccatctttacgtttctgggcactctgtgccaagaact |
| | | ggctggctttctgcctgggacgtcactggtttcccagaggtcctcccacatatgggtggtgggtaggtcag |
| | | agaagtcccactccagcatggctgcattgatcccccatcgttcccactagtctccgtaaaacctcccagat |
| | | acaggcacagtctagatgaaatcaggggtgcggggtgcaactgcaggccccaggcaattcaataggggctc |
| | | tactttcacccccaggtcaccccagaatgctcacacaccagacactgacgccctggggctgtcaagatcag |
| | | gcgtttgtctctgggcccagctcagggcccagctcagcacccactcagctcccctgaggctggggagcctg |
| | | tcccattgcgactggagaggagagcggggccacagaggcctggctagaaggtcccttctccctggtgtgtg |
| | | ttttctctctgctgagcaggcttgcagtgcctggggtatca |
| 4 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | noncleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | pA- | tccgccccaggattgggcgaagcctcccggctcgcactcgctcgcccgtgtgttccccgatcccgctggag |
| | 3′ homology | tcgatgcgcgtccagcgcgtgccaggccggggcgggggtgcgggctgactttctccctcgctagggacgct |
| | arm | ccggcgcccgaaaggaaagggtggcgctgcgctccggggtgcacgagccgacagcgcccgaccccaacggg |
| | | ccggccccgccagcgccgctaccgccctgcccccgggcgagcgggatgggcgggagtggagtggcgggtgg |
| | | agggtggagacgtcctggcccccgccccgcgtgcacccccaggggaggccgagcccgccgcccggccccgc |
| | | gcaggccccgcccgggactcccctgcggtccaggccgcgccccgggctccgcgccagccaatgagcgccgc |
| | | ccggccgggcgtgcccccgcgccccaagcataaaccctggcgcgctcgcggcccggcactcttctggtccc |
| | | cacagactcagagagaacccaccATGTACGGGAAGATTATTTTCGTGTTGTTGCTCAGTGAGATCGTTTCT |
| | | ATCTCCGCTGAAGACCCTCAAGGCGACGCCGCTCAAAAGACGGACACGAGCCATCACGACCAAGACCATCC |
| | | CACGTTTAATAAAATAACACCTAATCTCGCCGAATTTGCATTTTCACTGTATAGGCAACTCGCCCATCAAA |
| | | GCAATTCTACAAACATTTTCTTTAGCCCGGTCAGTATAGCGACTGCTTTCGCCATGCTGTCTCTCGGTACA |
| | | AAAGCCGATACCCATGACGAGATTTTGGAAGGACTCAACTTTAATCTGACCGAAATACCCGAAGCACAAAT |
| | | TCACGAGGGGTTTCAAGAGCTGCTGCGAACTTTGAATCAACCCGATTCCCAACTGCAACTCACGACAGGTA |
| | | ACGGGTTGTTTCTGAGTGAAGGGCTTAAACTGGTTGACAAATTCCTGGAAGACGTGAAGAAACTCTATCAT |
| | | AGTGAGGCATTTACAGTTAATTTTGGAGATACCGAGGAAGCTAAGAAGCAAATTAATGACTATGTTGAGAA |
| | | AGGCACGCAGGGAAAGATCGTTGACCTCGTGAAAGAACTGGATCGAGATACGGTGTTCGCCCTCGTCAACT |
| | | ATATATTCTTCAAGGGGAAGTGGGAACGCCCATTCGAGGTGAAAGATACAGAAGAGGAAGATTTTCATGTA |
| | | GATCAAGTTACAACAGTAAAAGTACCCATGATGAAAAGACTCGGGATGTTCAATATCCAACATTGCAAGAA |
| | | GTTGTCATCTTGGGTCCTTTTGATGAAGTATCTTGGGAACGCAACGGCTATATTCTTTCTCCCGGACGAAG |
| | | GCAAGCTGCAACATCTCGAGAACGAGCTGACTCATGACATTATTACGAAATTTCTTGAGAACGAGGATCGC |
| | | CGGAGCGCGTCCCTGCACCTGCCTAAGCTCAGCATAACAGGTACGTACGACCTCAAATCCGTGTTGGGACA |
| | | GTTGGGGATTACGAAAGTGTTTTCCAACGGAGCGGATCTGAGCGGTGTGACCGAAGAAGCTCCACTTAAAC |
| | | TGTCAAAAGCGGTCCACAAAGCCGTTTTGACTATAGATGAAAAGGGTACAGAGGCCGCCGGCGCGATGTTC |
| | | CTGGAAGCTATCCCGATGTCCATACCACCAGAAGTGAAATTTAATAAGCCTTTCGTGTTTCTGATGATAGA |
| | | GCAGAACACAAAATCCCCACTGTTTATGGGCAAGGTCGTCAACCCAACACAGAAGGGTTCAGGCGGGTCCG |
| | | GCGGAAGTGGGCTGATAATATTCGGCGTTATGGCCGGCGTGATCGGTACAATTCTGCTCATCAGTTATGGG |
| | | ATACGGAGATTGTGACTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCATCGCATTGTCTGAGTAG |
| | | GTGTCATTCTATTCTGGGGGGTGGGGTGGGGCAGGACAGCAAGGGGGAGGATTGGGAAGACAATAGCAGGC |
| | | ATGCTGGGGAtggccatgcttcttgccccttgggcctccccccagcccctcctccccttcctgcacccgta |
| | | cccccgtggtctttgaataaagtctgagtgggcggcagcctgtgtgtgcctgagttttttccctcagcaaa |
| | | cgtgccaggcatgggcgtggacagcagctgggacacacatggctagaacctctctgcagctggatagggta |
| | | ggaaaaggcaggggcgggaggaggggatggaggagggaaagtggagccaccgcgaagtccagctggaaaaa |
| | | cgctggaccctagagtgctttgaggatgcatttgctctttcccgagttttattcccagacttttcagattc |
| | | aatgcaggtttgctgaaataatgaatttatccatctttacgtttctgggcactctgtgccaagaactggct |
| | | ggctttctgcctgggacgtcactggtttcccagaggtcctcccacatatggggggggtaggtcagagaagt |
| | | cccactccagcatggctgcattgatcccccatcgttcccactagtctccgtaaaacctcccagatacaggc |
| | | acagtctagatgaaatcaggggtgcggggtgcaactgcaggccccaggcaattcaataggggctctacttt |
| | | cacccccaggtcaccccagaatgctcacacaccagacactgacgccctggggctgtcaagatcaggcgttt |
| | | gtctctgggcccagctcagggcccagctcagcacccactcagctcccctgaggctggggagcctgtcccat |
| | | tgcgactggagaggagagcggggccacagaggcctggctagaaggtcccttctccctggtgtgtgttttct |
| | | ctctgctgagcaggcttgcagtgcctggggtatca |
| 5 | 5′ homology | gctccagccggttccagctattgctttgtttacctgtttaaccagtatttacctagcaagtcttccatcag |
| | arm- - | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | noncleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | GPA(C-term)- | tccgccccaggattgggcgaagcctcccggctcgcactcgctcgcccgtgtgttccccgatcccgctggag |
| | 3′ homology | tcgatgcgcgtccagcgcgtgccaggccggggcgggggtgcgggctgactttctccctcgctagggacgct |
| | arm | ccggcgcccgaaaggaaagggtggcgctgcgctccggggtgcacgagccgacagcgcccgaccccaacggg |
| | | ccggccccgccagcgccgctaccgccctgcccccgggcgagcgggatggggggagtggagtggcgggtgga |
| | | gggtggagacgtcctggcccccgccccgcgtgcacccccaggggaggccgagcccgccgcccggccccgcg |
| | | caggccccgcccgggactcccctgcggtccaggccgcgccccgggctccgcgccagccaatgagcgccgcc |
| | | cggccgggcgtgcccccgcgccccaagcataaaccctggcgcgctcgcggcccggcactcttctggtcccc |
| | | acagactcagagagaacccaccATGTACGGGAAGATTATTTTCGTGTTGTTGCTCAGTGAGATCGTTTCTA |
| | | TCTCCGCTGAAGACCCTCAAGGCGACGCCGCTCAAAAGACGGACACGAGCCATCACGACCAAGACCATCCC |
| | | ACGTTTAATAAAATAACACCTAATCTCGCCGAATTTGCATTTTCACTGTATAGGCAACTCGCCCATCAAAG |
| | | CAATTCTACAAACATTTTCTTTAGCCCGGTCAGTATAGCGACTGCTTTCGCCATGCTGTCTCTCGGTACAA |
| | | AAGCCGATACCCATGACGAGATTTTGGAAGGACTCAACTTTAATCTGACCGAAATACCCGAAGCACAAATT |
| | | CACGAGGGGTTTCAAGAGCTGCTGCGAACTTTGAATCAACCCGATTCCCAACTGCAACTCACGACAGGTAA |
| | | CGGGTTGTTTCTGAGTGAAGGGCTTAAACTGGTTGACAAATTCCTGGAAGACGTGAAGAAACTCTATCATA |
| | | GTGAGGCATTTACAGTTAATTTTGGAGATACCGAGGAAGCTAAGAAGCAAATTAATGACTATGTTGAGAAA |
| | | GGCACGCAGGGAAAGATCGTTGACCTCGTGAAAGAACTGGATCGAGATACGGTGTTCGCCCTCGTCAACTA |
| | | TATATTCTTCAAGGGGAAGTGGGAACGCCCATTCGAGGTGAAAGATACAGAAGAGGAAGATTTTCATGTAG |
| | | ATCAAGTTACAACAGTAAAAGTACCCATGATGAAAAGACTCGGGATGTTCAATATCCAACATTGCAAGAAG |
| | | TTGTCATCTTGGGTCCTTTTGATGAAGTATCTTGGGAACGCAACGGCTATATTCTTTCTCCCGGACGAAGG |
| | | CAAGCTGCAACATCTCGAGAACGAGCTGACTCATGACATTATTACGAAATTTCTTGAGAACGAGGATCGCC |
| | | GGAGCGCGTCCCTGCACCTGCCTAAGCTCAGCATAACAGGTACGTACGACCTCAAATCCGTGTTGGGACAG |
| | | TTGGGGATTACGAAAGTGTTTTCCAACGGAGCGGATCTGAGCGGTGTGACCGAAGAAGCTCCACTTAAACT |
| | | GTCAAAAGCGGTCCACAAAGCCGTTTTGACTATAGATGAAAAGGGTACAGAGGCCGCCGGCGCGATGTTCC |
| | | TGGAAGCTATCCCGATGTCCATACCACCAGAAGTGAAATTTAATAAGCCTTTCGTGTTTCTGATGATAGAG |
| | | CAGAACACAAAATCCCCACTGTTTATGGGCAAGGTCGTCAACCCAACACAGAAGGGTTCAGGCGGGTCCGG |
| | | CGGAAGTGGGCTGATAATATTCGGCGTTATGGCCGGCGTGATCGGTACAATTCTGCTCATCAGTTATGGGA |
| | | TACGGAGATTGATCAAGAAGAGTCCTTCCGACGTCAAGCCACTGCCAAGCCCAGATACCGATGTTCCGCTT |
| | | TCTTCAGTGGAGATTGAGAACCCCGAAACTTCTGACCAGTGAtggccatgcttcttgccccttgggcctcc |
| | | ccccagcccctcctccccttcctgcacccgtacccccgtggtctttgaataaagtctgagtgggcggcagc |
| | | ctgtgtgtgcctgagttttttccctcagcaaacgtgccaggcatgggcgtggacagcagctgggacacaca |
| | | tggctagaacctctctgcagctggatagggtaggaaaaggcaggggcgggaggaggggatggaggagggaa |
| | | agtggagccaccgcgaagtccagctggaaaaacgctggaccctagagtgctttgaggatgcatttgctctt |
| | | tcccgagttttattcccagacttttcagattcaatgcaggtttgctgaaataatgaatttatccatcttta |
| | | cgtttctgggcactctgtgccaagaactggctggctttctgcctgggacgtcactggtttcccagaggtcc |
| | | tcccacatatgggtggtgggtaggtcagagaagtcccactccagcatggctgcattgatcccccatcgttc |
| | | ccactagtctccgtaaaacctcccagatacaggcacagtctagatgaaatcaggggtgcggggtgcaactg |
| | | caggccccaggcaattcaataggggctctactttcacccccaggtcaccccagaatgctcacacaccagac |
| | | actgacgccctggggctgtcaagatcaggcgtttgtctctgggcccagctcagggcccagctcagcaccca |
| | | ctcagctcccctgaggctggggagcctgtcccattgcgactggagaggagagcggggccacagaggcctgg |
| | | ctagaaggtcccttctccctggtgtgtgttttctctctgctgagcaggcttgcagtgcctggggtatca |
| 6 | 5′ homology | gctccagccggttccagctattgctttgtttacctgtttaaccagtatttacctagcaagtcttccatcag |
| | arm- - | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | noncleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | GPA(C-term)- | tccgccccaggattgggcgaagcctcccggctcgcactcgctcgcccgtgtgttccccgatcccgctggag |
| | pA- | tcgatgcgcgtccagcgcgtgccaggccggggcgggggtgcgggctgactttctccctcgctagggacgct |
| | 3′ homology | ccggcgcccgaaaggaaagggtggcgctgcgctccggggtgcacgagccgacagcgcccgaccccaacggg |
| | arm | ccggccccgccagcgccgctaccgccctgcccccgggcgagcgggatggggggagtggagtggcgggtgga |
| | | gggtggagacgtcctggcccccgccccgcgtgcacccccaggggaggccgagcccgccgcccggccccgcg |
| | | caggccccgcccgggactcccctgcggtccaggccgcgccccgggctccgcgccagccaatgagcgccgcc |
| | | cggccgggcgtgcccccgcgccccaagcataaaccctggcgcgctcgcggcccggcactcttctggtcccc |
| | | acagactcagagagaacccaccATGTACGGGAAGATTATTTTCGTGTTGTTGCTCAGTGAGATCGTTTCTA |
| | | TCTCCGCTGAAGACCCTCAAGGCGACGCCGCTCAAAAGACGGACACGAGCCATCACGACCAAGACCATCCC |
| | | ACGTTTAATAAAATAACACCTAATCTCGCCGAATTTGCATTTTCACTGTATAGGCAACTCGCCCATCAAAG |
| | | CAATTCTACAAACATTTTCTTTAGCCCGGTCAGTATAGCGACTGCTTTCGCCATGCTGTCTCTCGGTACAA |
| | | AAGCCGATACCCATGACGAGATTTTGGAAGGACTCAACTTTAATCTGACCGAAATACCCGAAGCACAAATT |
| | | CACGAGGGGTTTCAAGAGCTGCTGCGAACTTTGAATCAACCCGATTCCCAACTGCAACTCACGACAGGTAA |
| | | CGGGTTGTTTCTGAGTGAAGGGCTTAAACTGGTTGACAAATTCCTGGAAGACGTGAAGAAACTCTATCATA |
| | | GTGAGGCATTTACAGTTAATTTTGGAGATACCGAGGAAGCTAAGAAGCAAATTAATGACTATGTTGAGAAA |
| | | GGCACGCAGGGAAAGATCGTTGACCTCGTGAAAGAACTGGATCGAGATACGGTGTTCGCCCTCGTCAACTA |
| | | TATATTCTTCAAGGGGAAGTGGGAACGCCCATTCGAGGTGAAAGATACAGAAGAGGAAGATTTTCATGTAG |
| | | ATCAAGTTACAACAGTAAAAGTACCCATGATGAAAAGACTCGGGATGTTCAATATCCAACATTGCAAGAAG |
| | | TTGTCATCTTGGGTCCTTTTGATGAAGTATCTTGGGAACGCAACGGCTATATTCTTTCTCCCGGACGAAGG |
| | | CAAGCTGCAACATCTCGAGAACGAGCTGACTCATGACATTATTACGAAATTTCTTGAGAACGAGGATCGCC |
| | | GGAGCGCGTCCCTGCACCTGCCTAAGCTCAGCATAACAGGTACGTACGACCTCAAATCCGTGTTGGGACAG |
| | | TTGGGGATTACGAAAGTGTTTTCCAACGGAGCGGATCTGAGCGGTGTGACCGAAGAAGCTCCACTTAAACT |
| | | GTCAAAAGCGGTCCACAAAGCCGTTTTGACTATAGATGAAAAGGGTACAGAGGCCGCCGGCGCGATGTTCC |
| | | TGGAAGCTATCCCGATGTCCATACCACCAGAAGTGAAATTTAATAAGCCTTTCGTGTTTCTGATGATAGAG |
| | | CAGAACACAAAATCCCCACTGTTTATGGGCAAGGTCGTCAACCCAACACAGAAGGGTTCAGGCGGGTCCGG |
| | | CGGAAGTGGGCTGATAATATTCGGCGTTATGGCCGGCGTGATCGGTACAATTCTGCTCATCAGTTATGGGA |
| | | TACGGAGATTGATCAAGAAGAGTCCTTCCGACGTCAAGCCACTGCCAAGCCCAGATACCGATGTTCCGCTT |
| | | TCTTCAGTGGAGATTGAGAACCCCGAAACTTCTGACCAGTGACTGTGCCTTCTAGTTGCCAGCCATCTGTT |
| | | GTTTGCCCCTCCCCCGTGCCTTCCTTGACCCTGGAAGGTGCCACTCCCACTGTCCTTTCCTAATAAAATGA |
| | | GGAAATTGCATCGCATTGTCTGAGTAGGTGTCATTCTATTCTGGGGGGTGGGGTGGGGCAGGACAGCAAGG |
| | | GGGAGGATTGGGAAGACAATAGCAGGCATGCTGGGGAtggccatgcttcttgccccttgggcctcccccca |
| | | gcccctcctccccttcctgcacccgtacccccgtggtctttgaataaagtctgagtgggcggcagcctgtg |
| | | tgtgcctgagttttttccctcagcaaacgtgccaggcatgggcgtggacagcagctgggacacacatggct |
| | | agaacctctctgcagctggatagggtaggaaaaggcaggggcgggaggaggggatggaggagggaaagtgg |
| | | agccaccgcgaagtccagctggaaaaacgctggaccctagagtgctttgaggatgcatttgctctttcccg |
| | | agttttattcccagacttttcagattcaatgcaggtttgctgaaataatgaatttatccatctttacgttt |
| | | ctgggcactctgtgccaagaactggctggctttctgcctgggacgtcactggtttcccagaggtcctccca |
| | | catatgggtggtgggtaggtcagagaagtcccactccagcatggctgcattgatcccccatcgttcccact |
| | | agtctccgtaaaacctcccagatacaggcacagtctagatgaaatcaggggtgcggggtgcaactgcaggc |
| | | cccaggcaattcaataggggctctactttcacccccaggtcaccccagaatgctcacacaccagacactga |
| | | cgccctggggctgtcaagatcaggcgtttgtctctgggcccagctcagggcccagctcagcacccactcag |
| | | ctcccctgaggctggggagcctgtcccattgcgactggagaggagagcggggccacagaggcctggctaga |
| | | aggtcccttctccctggtgtgtgttttctctctgctgagcaggcttgcagtgcctggggtatca |
| 7 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctgtgcaggaagcgaggctg |
| | cleavable | gagagcaggaggggctctgcgcagaaattcttttgagttcctatgggcgtgtttattccttcccggtgcct |
| | linker-GPA- | gtcactcaagcacactagtgactatcgccagagggaaagggagccagggcgtccgggtgcgcgcattcctc |
| | 3′ homology | tccgccccaggattgggcgaagcctcccggctcgcactcgctcgcccgtgtgttccccgatcccgctggag |
| | arm | tcgatgcgcgtccagcgcgtgccaggccggggcgggggtgcgggctgactttctccctcgctagggacgct |
| | | ccggcgcccgaaaggaaagggtggcgctgcgctccggggtgcacgagccgacagcgcccgaccccaacggg |
| | | ccggccccgccagcgccgctaccgccctgcccccgggcgagcgggatgggcgggagtggagtggcgggtgg |
| | | agggtggagacgtcctggcccccgccccgcgtgcacccccaggggaggccgagcccgccgcccggccccgc |
| | | gcaggccccgcccgggactcccctgcggtccaggccgcgccccgggctccgcgccagccaatgagcgccgc |
| | | ccggccgggcgtgcccccgcgccccaagcataaaccctggcgcgctcgcggcccggcactcttctggtccc |
| | | cacagactcagagagaacccaccATGTACGGGAAGATTATTTTCGTGTTGTTGCTCAGTGAGATCGTTTCT |
| | | ATCTCCGCTGAAGACCCTCAAGGCGACGCCGCTCAAAAGACGGACACGAGCCATCACGACCAAGACCATCC |
| | | CACGTTTAATAAAATAACACCTAATCTCGCCGAATTTGCATTTTCACTGTATAGGCAACTCGCCCATCAAA |
| | | GCAATTCTACAAACATTTTCTTTAGCCCGGTCAGTATAGCGACTGCTTTCGCCATGCTGTCTCTCGGTACA |
| | | AAAGCCGATACCCATGACGAGATTTTGGAAGGACTCAACTTTAATCTGACCGAAATACCCGAAGCACAAAT |
| | | TCACGAGGGGTTTCAAGAGCTGCTGCGAACTTTGAATCAACCCGATTCCCAACTGCAACTCACGACAGGTA |
| | | ACGGGTTGTTTCTGAGTGAAGGGCTTAAACTGGTTGACAAATTCCTGGAAGACGTGAAGAAACTCTATCAT |
| | | AGTGAGGCATTTACAGTTAATTTTGGAGATACCGAGGAAGCTAAGAAGCAAATTAATGACTATGTTGAGAA |
| | | AGGCACGCAGGGAAAGATCGTTGACCTCGTGAAAGAACTGGATCGAGATACGGTGTTCGCCCTCGTCAACT |
| | | ATATATTCTTCAAGGGGAAGTGGGAACGCCCATTCGAGGTGAAAGATACAGAAGAGGAAGATTTTCATGTA |
| | | GATCAAGTTACAACAGTAAAAGTACCCATGATGAAAAGACTCGGGATGTTCAATATCCAACATTGCAAGAA |
| | | GTTGTCATCTTGGGTCCTTTTGATGAAGTATCTTGGGAACGCAACGGCTATATTCTTTCTCCCGGACGAAG |
| | | GCAAGCTGCAACATCTCGAGAACGAGCTGACTCATGACATTATTACGAAATTTCTTGAGAACGAGGATCGC |
| | | CGGAGCGCGTCCCTGCACCTGCCTAAGCTCAGCATAACAGGTACGTACGACCTCAAATCCGTGTTGGGACA |
| | | GTTGGGGATTACGAAAGTGTTTTCCAACGGAGCGGATCTGAGCGGTGTGACCGAAGAAGCTCCACTTAAAC |
| | | TGTCAAAAGCGGTCCACAAAGCCGTTTTGACTATAGATGAAAAGGGTACAGAGGCCGCCGGCGCGATGTTC |
| | | CTGGAAGCTATCCCGATGTCCATACCACCAGAAGTGAAATTTAATAAGCCTTTCGTGTTTCTGATGATAGA |
| | | GCAGAACACAAAATCCCCACTGTTTATGGGCAAGGTCGTCAACCCAACACAGAAGGGTTCAGGCGGGTCCG |
| | | GCGGAAGTGGGCCACTTGGTATGTGGTCTAGGCTGATAATATTCGGCGTTATGGCCGGCGTGATCGGTACA |
| | | ATTCTGCTCATCAGTTATGGGATACGGAGATTGTGAtggccatgcttcttgccccttgggcctccccccag |
| | | cccctcctccccttcctgcacccgtacccccgtggtctttgaataaagtctgagtgggcggcagcctgtgt |
| | | gtgcctgagttttttccctcagcaaacgtgccaggcatgggcgtggacagcagctgggacacacatggcta |
| | | gaacctctctgcagctggatagggtaggaaaaggcaggggcgggaggaggggatggaggagggaaagtgga |
| | | gccaccgcgaagtccagctggaaaaacgctggaccctagagtgctttgaggatgcatttgctctttcccga |
| | | gttttattcccagacttttcagattcaatgcaggtttgctgaaataatgaatttatccatctttacgtttc |
| | | tgggcactctgtgccaagaactggctggctttctgcctgggacgtcactggtttcccagaggtcctcccac |
| | | atatggggggggtaggtcagagaagtcccactccagcatggctgcattgatcccccatcgttcccactagt |
| | | ctccgtaaaacctcccagatacaggcacagtctagatgaaatcaggggtgcggggtgcaactgcaggcccc |
| | | aggcaattcaataggggctctactttcacccccaggtcaccccagaatgctcacacaccagacactgacgc |
| | | cctggggctgtcaagatcaggcgtttgtctctgggcccagctcagggcccagctcagcacccactcagctc |
| | | ccctgaggctggggagcctgtcccattgcgactggagaggagagcggggccacagaggcctggctagaagg |
| | | tcccttctccctggtgtgtgttttctctctgctgagcaggcttgcagtgcctggggtatca |
| 8 | 5′ homology | gctccagccggttccagctattgctttgtttacctgtttaaccagtatttacctagcaagtcttccatcag |
| | arm- | atagcatttggagagctgggggtgtcacagtgaaccacgacctctaggccagtgggagagtcagtcacaca |
| | therapeutic | aactgtgagtccatgacttggggcttagccagcacccaccaccccacgcgccaccccacaaccccgggtag |
| | protein- | aggagtctgaatctggagccgcccccagcccagccccgtgctttttgcgtcctggtgtttattccttcccg |
| | cleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | pA- | tccgccccaggattgggcgaagcctcccggctcgcactcgctcgcccgtgtgttccccgatcccgctggag |
| | 3′ homology | tcgatgcgcgtccagcgcgtgccaggccggggcgggggtgcgggctgactttctccctcgctagggacgct |
| | arm | ccggcgcccgaaaggaaagggtggcgctgcgctccggggtgcacgagccgacagcgcccgaccccaacggg |
| | | ccggccccgccagcgccgctaccgccctgcccccgggcgagcgggatgggcgggagtggagtggcgggtgg |
| | | agggtggagacgtcctggcccccgccccgcgtgcacccccaggggaggccgagcccgccgcccggccccgc |
| | | gcaggccccgcccgggactcccctgcggtccaggccgcgccccgggctccgcgccagccaatgagcgccgc |
| | | ccggccgggcgtgcccccgcgccccaagcataaaccctggcgcgctcgcggcccggcactcttctggtccc |
| | | cacagactcagagagaacccaccATGTACGGGAAGATTATTTTCGTGTTGTTGCTCAGTGAGATCGTTTCT |
| | | ATCTCCGCTGAAGACCCTCAAGGCGACGCCGCTCAAAAGACGGACACGAGCCATCACGACCAAGACCATCC |
| | | CACGTTTAATAAAATAACACCTAATCTCGCCGAATTTGCATTTTCACTGTATAGGCAACTCGCCCATCAAA |
| | | GCAATTCTACAAACATTTTCTTTAGCCCGGTCAGTATAGCGACTGCTTTCGCCATGCTGTCTCTCGGTACA |
| | | AAAGCCGATACCCATGACGAGATTTTGGAAGGACTCAACTTTAATCTGACCGAAATACCCGAAGCACAAAT |
| | | TCACGAGGGGTTTCAAGAGCTGCTGCGAACTTTGAATCAACCCGATTCCCAACTGCAACTCACGACAGGTA |
| | | ACGGGTTGTTTCTGAGTGAAGGGCTTAAACTGGTTGACAAATTCCTGGAAGACGTGAAGAAACTCTATCAT |
| | | AGTGAGGCATTTACAGTTAATTTTGGAGATACCGAGGAAGCTAAGAAGCAAATTAATGACTATGTTGAGAA |
| | | AGGCACGCAGGGAAAGATCGTTGACCTCGTGAAAGAACTGGATCGAGATACGGTGTTCGCCCTCGTCAACT |
| | | ATATATTCTTCAAGGGGAAGTGGGAACGCCCATTCGAGGTGAAAGATACAGAAGAGGAAGATTTTCATGTA |
| | | GATCAAGTTACAACAGTAAAAGTACCCATGATGAAAAGACTCGGGATGTTCAATATCCAACATTGCAAGAA |
| | | GTTGTCATCTTGGGTCCTTTTGATGAAGTATCTTGGGAACGCAACGGCTATATTCTTTCTCCCGGACGAAG |
| | | GCAAGCTGCAACATCTCGAGAACGAGCTGACTCATGACATTATTACGAAATTTCTTGAGAACGAGGATCGC |
| | | CGGAGCGCGTCCCTGCACCTGCCTAAGCTCAGCATAACAGGTACGTACGACCTCAAATCCGTGTTGGGACA |
| | | GTTGGGGATTACGAAAGTGTTTTCCAACGGAGCGGATCTGAGCGGTGTGACCGAAGAAGCTCCACTTAAAC |
| | | TGTCAAAAGCGGTCCACAAAGCCGTTTTGACTATAGATGAAAAGGGTACAGAGGCCGCCGGCGCGATGTTC |
| | | CTGGAAGCTATCCCGATGTCCATACCACCAGAAGTGAAATTTAATAAGCCTTTCGTGTTTCTGATGATAGA |
| | | GCAGAACACAAAATCCCCACTGTTTATGGGCAAGGTCGTCAACCCAACACAGAAGGGTTCAGGCGGGTCCG |
| | | GCGGAAGTGGGCCACTTGGTATGTGGTCTAGGCTGATAATATTCGGCGTTATGGCCGGCGTGATCGGTACA |
| | | ATTCTGCTCATCAGTTATGGGATACGGAGATTGTGACTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGGAAAT |
| | | TGCATCGCATTGTCTGAGTAGGTGTCATTCTATTCTGGGGGGTGGGGTGGGGCAGGACAGCAAGGGGGAGG |
| | | ATTGGGAAGACAATAGCAGGCATGCTGGGGAtggccatgcttcttgccccttgggcctccccccagcccct |
| | | cctccccttcctgcacccgtacccccgtggtctttgaataaagtctgagtgggcggcagcctgtgtgtgcc |
| | | tgagttttttccctcagcaaacgtgccaggcatgggcgtggacagcagctgggacacacatggctagaacc |
| | | tctctgcagctggatagggtaggaaaaggcaggggcgggaggaggggatggaggagggaaagtggagccac |
| | | cgcgaagtccagctggaaaaacgctggaccctagagtgctttgaggatgcatttgctctttcccgagtttt |
| | | attcccagacttttcagattcaatgcaggtttgctgaaataatgaatttatccatctttacgtttctgggc |
| | | actctgtgccaagaactggctggctttctgcctgggacgtcactggtttcccagaggtcctcccacatatg |
| | | ggtggtgggtaggtcagagaagtcccactccagcatggctgcattgatcccccatcgttcccactagtctc |
| | | cgtaaaacctcccagatacaggcacagtctagatgaaatcaggggtgcggggtgcaactgcaggccccagg |
| | | caattcaataggggctctactttcacccccaggtcaccccagaatgctcacacaccagacactgacgccct |
| | | ggggctgtcaagatcaggcgtttgtctctgggcccagctcagggcccagctcagcacccactcagctcccc |
| | | tgaggctggggagcctgtcccattgcgactggagaggagagcggggccacagaggcctggctagaaggtcc |
| | | cttctccctggtgtgtgttttctctctgctgagcaggcttgcagtgcctggggtatca |
| 9 | 5′ homology | gctccagccggttccagctattgctttgtttacctgtttaaccagtatttacctagcaagtcttccatcag |
| | arm- - | atagcatttggagagctgggggtgtcacagtgaaccacgacctctaggccagtgggagagtcagtcacaca |
| | therapeutic | aactgtgagtccatgacttggggcttagccagcacccaccaccccacgegccaccccacaaccccgggtag |
| | protein- | aggagtctgaatctggagccgcccccagcccagccccgtgctttttgcgtcctggtgtttattccttcccg |
| | cleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | GPA(C- | tccgccccaggattgggcgaagcctcccggctcgcactcgctcgcccgtgtgttccccgatcccgctggag |
| | term)- | tcgatgcgcgtccagcgcgtgccaggccggggcgggggtgcgggctgactttctccctcgctagggacgct |
| | 3′ homology | ccggcgcccgaaaggaaagggtggcgctgcgctccggggtgcacgagccgacagcgcccgaccccaacggg |
| | arm | ccggccccgccagcgccgctaccgccctgcccccgggcgagcgggatgggcgggagtggagtggcgggtgg |
| | | agggtggagacgtcctggcccccgccccgcgtgcacccccaggggaggccgagccegccgcceggccccgc |
| | | gcaggccccgcccgggactcccctgcggtccaggcegcgccccgggctcegcgccagccaatgagcgccgc |
| | | ccggccgggcgtgcccccgegccccaagcataaaccctggcgcgctcgcggcccggcactcttctggtccc |
| | | cacagactcagagagaacccaccATGTACGGGAAGATTATTTTCGTGTTGTTGCTCAGTGAGATCGTTTCT |
| | | ATCTCCGCTGAAGACCCTCAAGGCGACGCCGCTCAAAAGACGGACACGAGCCATCACGACCAAGACCATCC |
| | | CACGTTTAATAAAATAACACCTAATCTCGCCGAATTTGCATTTTCACTGTATAGGCAACTCGCCCATCAAA |
| | | GCAATTCTACAAACATTTTCTTTAGCCCGGTCAGTATAGCGACTGCTTTCGCCATGCTGTCTCTCGGTACA |
| | | AAAGCCGATACCCATGACGAGATTTTGGAAGGACTCAACTTTAATCTGACCGAAATACCCGAAGCACAAAT |
| | | TCACGAGGGGTTTCAAGAGCTGCTGCGAACTTTGAATCAACCCGATTCCCAACTGCAACTCACGACAGGTA |
| | | ACGGGTTGTTTCTGAGTGAAGGGCTTAAACTGGTTGACAAATTCCTGGAAGACGTGAAGAAACTCTATCAT |
| | | AGTGAGGCATTTACAGTTAATTTTGGAGATACCGAGGAAGCTAAGAAGCAAATTAATGACTATGTTGAGAA |
| | | AGGCACGCAGGGAAAGATCGTTGACCTCGTGAAAGAACTGGATCGAGATACGGTGTTCGCCCTCGTCAACT |
| | | ATATATTCTTCAAGGGGAAGTGGGAACGCCCATTCGAGGTGAAAGATACAGAAGAGGAAGATTTTCATGTA |
| | | GATCAAGTTACAACAGTAAAAGTACCCATGATGAAAAGACTCGGGATGTTCAATATCCAACATTGCAAGAA |
| | | GTTGTCATCTTGGGTCCTTTTGATGAAGTATCTTGGGAACGCAACGGCTATATTCTTTCTCCCGGACGAAG |
| | | GCAAGCTGCAACATCTCGAGAACGAGCTGACTCATGACATTATTACGAAATTTCTTGAGAACGAGGATCGC |
| | | CGGAGCGCGTCCCTGCACCTGCCTAAGCTCAGCATAACAGGTACGTACGACCTCAAATCCGTGTTGGGACA |
| | | GTTGGGGATTACGAAAGTGTTTTCCAACGGAGCGGATCTGAGCGGTGTGACCGAAGAAGCTCCACTTAAAC |
| | | TGTCAAAAGCGGTCCACAAAGCCGTTTTGACTATAGATGAAAAGGGTACAGAGGCCGCCGGCGCGATGTTC |
| | | CTGGAAGCTATCCCGATGTCCATACCACCAGAAGTGAAATTTAATAAGCCTTTCGTGTTTCTGATGATAGA |
| | | GCAGAACACAAAATCCCCACTGTTTATGGGCAAGGTCGTCAACCCAACACAGAAGGGTTCAGGCGGGTCCG |
| | | GCGGAAGTGGGCCACTTGGTATGTGGTCTAGGCTGATAATATTCGGCGTTATGGCCGGCGTGATCGGTACA |
| | | ATTCTGCTCATCAGTTATGGGATACGGAGATTGATCAAGAAGAGTCCTTCCGACGTCAAGCCACTGCCAAG |
| | | CCCAGATACCGATGTTCCGCTTTCTTCAGTGGAGATTGAGAACCCCGAAACTTCTGACCAGTGAtggccat |
| | | gcttcttgccccttgggcctccccccagcccctectccccttcctgcacccgtacccccgtggtctttgaa |
| | | taaagtctgagtgggcggcagcctgtgtgtgcctgagttttttccctcagcaaacgtgccaggcatgggcg |
| | | tggacagcagctgggacacacatggctagaacctctctgcagctggatagggtaggaaaaggcaggggcgg |
| | | gaggaggggatggaggagggaaagtggagccaccgcgaagtccagctggaaaaacgctggaccctagagtg |
| | | ctttgaggatgcatttgctctttcccgagttttattcccagacttttcagattcaatgcaggtttgctgaa |
| | | ataatgaatttatccatctttacgtttctgggcactctgtgccaagaactggctggctttctgcctgggac |
| | | gtcactggtttcccagaggtcctcccacatatgggtggtgggtaggtcagagaagtcccactccagcatgg |
| | | ctgcattgatcccccatcgttcccactagtctccgtaaaacctcccagatacaggcacagtctagatgaaa |
| | | tcaggggtgcggggtgcaactgcaggccccaggcaattcaataggggctctactttcacccccaggtcacc |
| | | ccagaatgctcacacaccagacactgacgccctggggctgtcaagatcaggcgtttgtctctgggcccagc |
| | | tcagggcccagctcagcacccactcagctcccctgaggctggggagcctgtcccattgcgactggagagga |
| | | gagcggggccacagaggcctggctagaaggtcccttctccctggtgtgtgttttctctctgctgagcaggc |
| | | ttgcagtgcctggggtatca |
| 10 | 5′ homology | gctccagccggttccagctattgctttgtttacctgtttaaccagtatttacctagcaagtcttccatcag |
| | arm- - | atagcatttggagagctgggggtgtcacagtgaaccacgacctctaggccagtgggagagtcagtcacaca |
| | therapeutic | aactgtgagtccatgacttggggcttagccagcacccaccaccccacgegccaccccacaaccccgggtag |
| | protein- | aggagtctgaatctggagccgcccccagcccagccccgtgctttttgcgtcctggtgtttattccttcccg |
| | cleavable | gtgcctgtcactcaagcacactagtgactatcgccagagggaaagggagctgcaggaagcgaggctggaga |
| | linker-GPA- | gcaggaggggctctgcgcagaaattcttttgagttcctatgggccagggcgtccgggtgcgcgcattcctc |
| | GPA(C-term)- | tccgccccaggattgggcgaagcctcccggctegcactcgctcgcccgtgtgttccccgatcccgctggag |
| | pA- | tcgatgcgcgtccagcgcgtgccaggccggggcgggggtgcgggctgactttctccctcgctagggacgct |
| | 3′ homology | ccggcgcccgaaaggaaagggtggcgctgcgctccggggtgcacgagccgacagcgcccgaccccaacggg |
| | arm | ccggccccgccagcgccgctaccgccctgcccccgggcgagcgggatgggcgggagtggagtggcgggtgg |
| | | agggtggagacgtcctggcccccgccccgcgtgcacccccaggggaggccgagcccgccgcceggccccgc |
| | | gcaggccccgcccgggactcccctgcggtccaggcegcgccccgggctcegcgccagccaatgagcgccgc |
| | | ccggccgggcgtgcccccgcgccccaagcataaaccctggcgcgctcgcggcccggcactcttctggtccc |
| | | cacagactcagagagaacccaccATGTACGGGAAGATTATTTTCGTGTTGTTGCTCAGTGAGATCGTTTCT |
| | | ATCTCCGCTGAAGACCCTCAAGGCGACGCCGCTCAAAAGACGGACACGAGCCATCACGACCAAGACCATCC |
| | | CACGTTTAATAAAATAACACCTAATCTCGCCGAATTTGCATTTTCACTGTATAGGCAACTCGCCCATCAAA |
| | | GCAATTCTACAAACATTTTCTTTAGCCCGGTCAGTATAGCGACTGCTTTCGCCATGCTGTCTCTCGGTACA |
| | | AAAGCCGATACCCATGACGAGATTTTGGAAGGACTCAACTTTAATCTGACCGAAATACCCGAAGCACAAAT |
| | | TCACGAGGGGTTTCAAGAGCTGCTGCGAACTTTGAATCAACCCGATTCCCAACTGCAACTCACGACAGGTA |
| | | ACGGGTTGTTTCTGAGTGAAGGGCTTAAACTGGTTGACAAATTCCTGGAAGACGTGAAGAAACTCTATCAT |
| | | AGTGAGGCATTTACAGTTAATTTTGGAGATACCGAGGAAGCTAAGAAGCAAATTAATGACTATGTTGAGAA |
| | | AGGCACGCAGGGAAAGATCGTTGACCTCGTGAAAGAACTGGATCGAGATACGGTGTTCGCCCTCGTCAACT |
| | | ATATATTCTTCAAGGGGAAGTGGGAACGCCCATTCGAGGTGAAAGATACAGAAGAGGAAGATTTTCATGTA |
| | | GATCAAGTTACAACAGTAAAAGTACCCATGATGAAAAGACTCGGGATGTTCAATATCCAACATTGCAAGAA |
| | | GTTGTCATCTTGGGTCCTTTTGATGAAGTATCTTGGGAACGCAACGGCTATATTCTTTCTCCCGGACGAAG |
| | | GCAAGCTGCAACATCTCGAGAACGAGCTGACTCATGACATTATTACGAAATTTCTTGAGAACGAGGATCGC |
| | | CGGAGCGCGTCCCTGCACCTGCCTAAGCTCAGCATAACAGGTACGTACGACCTCAAATCCGTGTTGGGACA |
| | | GTTGGGGATTACGAAAGTGTTTTCCAACGGAGCGGATCTGAGCGGTGTGACCGAAGAAGCTCCACTTAAAC |
| | | TGTCAAAAGCGGTCCACAAAGCCGTTTTGACTATAGATGAAAAGGGTACAGAGGCCGCCGGCGCGATGTTC |
| | | CTGGAAGCTATCCCGATGTCCATACCACCAGAAGTGAAATTTAATAAGCCTTTCGTGTTTCTGATGATAGA |
| | | GCAGAACACAAAATCCCCACTGTTTATGGGCAAGGTCGTCAACCCAACACAGAAGGGTTCAGGCGGGTCCG |
| | | GCGGAAGTGGGCCACTTGGTATGTGGTCTAGGCTGATAATATTCGGCGTTATGGCCGGCGTGATCGGTACA |
| | | ATTCTGCTCATCAGTTATGGGATACGGAGATTGATCAAGAAGAGTCCTTCCGACGTCAAGCCACTGCCAAG |
| | | CCCAGATACCGATGTTCCGCTTTCTTCAGTGGAGATTGAGAACCCCGAAACTTCTGACCAGTGACTGTGCC |
| | | TTCTAGTTGCCAGCCATCTGTTGTTTGCCCCTCCCCCGTGCCTTCCTTGACCCTGGAAGGTGCCACTCCCA |
| | | CTGTCCTTTCCTAATAAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTGGGGAtggccatgcttc |
| | | ttgccccttgggcctccccccagcccctectccccttcctgcacccgtacccccgtggtctttgaataaag |
| | | tctgagtgggcggcagcctgtgtgtgcctgagttttttccctcagcaaacgtgccaggcatgggcgtggac |
| | | agcagctgggacacacatggctagaacctctctgcagctggatagggtaggaaaaggcaggggcgggagga |
| | | ggggatggaggagggaaagtggagccaccgcgaagtccagctggaaaaacgctggaccctagagtgctttg |
| | | aggatgcatttgctctttcccgagttttattcccagacttttcagattcaatgcaggtttgctgaaataat |
| | | gaatttatccatctttacgtttctgggcactctgtgccaagaactggctggctttctgcctgggacgtcac |
| | | tggtttcccagaggtcctcccacatatggggggggtaggtcagagaagtcccactccagcatggctgcatt |
| | | gatcccccatcgttcccactagtctccgtaaaacctcccagatacaggcacagtctagatgaaatcagggg |
| | | tgcggggtgcaactgcaggccccaggcaattcaataggggctctactttcacccccaggtcaccccagaat |
| | | gctcacacaccagacactgacgccctggggctgtcaagatcaggcgtttgtctctgggcccagctcagggc |
| | | ccagctcagcacccactcagctcccctgaggctggggagcctgtcccattgcgactggagaggagagcggg |
| | | gccacagaggcctggctagaaggtcccttctccctggtgtgtgttttctctctgctgagcaggcttgcagt |
| | | gcctggggtatca |
| 11 | 5′ homology | GCTTTCATGAATTCCCCCAACAGAGCCAAGCTCTCCATCTAGTGGACAGGGAAGCTAGCAGCAAACCTTCC |
| | arm- | CTTCACTACAAAACTTCATTGCTTGGCCAAAAAGAGAGTTAATTCAATGTAGACATCTATGTAGGCAATTA |
| | promoter- | AAAACCTATTGATGTATAAAACAGTTTGCATTCATGGAGGGCAACTAAATACATTCTAGGACTTTATAAAA |
| | therapeutic | GATCACTTTTTATTTATGCACAGGGTGGAACAAGATGGATTATCAAGTGTCAAGTCCAATCTATGACATCA |
| | protein-pA- | ATTATTATACATCGGAGCCCTGCCAAAAAATCAATGTGAAGCAAATCGCAGCCCGCCTCCTGCCTCCGCTC |
| | 3′ homology | TACTCACTGGTGTTCATCTTTGGTTTTGTGGGCAACATGCTGGTCATCCTCATCCTGATAAACTGCAAAAG |
| | arm | GCTGAAGAGCATGACTGACATCTACCTGCTCAACCTGGCCATCTCTGACCTGTTTTTCCTTCTTACTGTCC |
| | | CCTTCGAACCTCAGAACACTCAAATGATTTAAATTTCTCAAATACATTCATTTCACATATAGGAAGTCACT |
| | | TTCATTTGGACCACTGGGTCTTGACATTAGAAATGAGAAGGTCCATGGCTCCACAACAGCTACCTCAGCCT |
| | | GGCACGTGCCCTGGCCTCAGAGATTCACAGTCCAGTTCTTTGTCCAGTTGGGTGGCTCCTGTCTACCACCT |
| | | TACCATGCCCACTTAACTGATGCAAAGTTAATATCACAAGTAGCAACCTGTTCCTTGCAGTGAAAATTTTA |
| | | CTTACCACTTTCATAGCCCCAAGATATCCATGTATCTTTATTAACAGGCGCTTAACAACTTGCATCATTTA |
| | | AAATGCCTCCCCTGCCTATCAGCTGATGATGGCCGCAGGAAGGTGGGCCTGGAAGATAACAGCTAGCAGGC |
| | | TAAGGTCAGACACTGACACTTGCAGTTGTCTTTGGTAGTTTTTTTGCACTAACTTCAGGAACCAGCTCATG |
| | | ATCTCAGGATGCCTTCATCAGTATCTTGGGGAATACTGCTCCTTGCTGGGTTGTGTTGTCTCGTACCCGTG |
| | | AGTCTCGCCGAAGACCCTCAAGGCGACGCCGCACAAAAGACTGACACTTCTCATCACGACCAAGACCATCC |
| | | TACATTTAATAAAATTACTCCAAATCTCGCCGAATTTGCGTTTTCTCTGTATAGGCAACTCGCTCACCAAT |
| | | CTAATTCAACGAACATATTCTTTTCACCTGTTTCCATAGCCACCGCTTTCGCCATGCTGAGTTTGGGAACA |
| | | AAAGCAGATACCCATGACGAGATACTCGAAGGACTCAACTTTAATCTGACAGAAATCCCTGAAGCACAAAT |
| | | TCACGAGGGTTTTCAAGAGCTGCTGAGAACTTTGAATCAACCCGATTCCCAATTGCAACTCACAACAGGAA |
| | | ACGGTTTGTTTCTTTCAGAAGGGCTCAAACTGGTCGACAAATTCCTCGAAGACGTGAAGAAACTTTATCAT |
| | | AGCGAGGCTTTTACCGTGAATTTTGGAGATACGGAAGAAGCTAAGAAGCAAATAAATGACTATGTCGAAAA |
| | | GGGGACACAGGGAAAGATAGTTGACCTGGTGAAAGAACTGGATAGGGATACTGTGTTCGCGCTCGTCAACT |
| | | ATATCTTCTTCAAGGGGAAGTGGGAACGGCCATTCGAGGTTAAAGATACAGAAGAGGAAGATTTTCATGTA |
| | | GATCAAGTCACAACAGTCAAAGTTCCAATGATGAAACGCCTCGGGATGTTCAATATACAACATTGCAAGAA |
| | | ACTTAGCTCATGGGTCCTTTTGATGAAGTATCTCGGGAACGCTACAGCGATATTCTTTCTCCCAGACGAAG |
| | | GTAAGCTGCAACATCTTGAGAACGAGCTGACACATGACATAATAACAAAATTTCTTGAGAACGAGGATCGC |
| | | CGGTCCGCATCCCTGCACCTGCCGAAGCTTAGCATAACCGGCACATACGACTTGAAATCTGTTCTTGGGCA |
| | | GCTTGGTATTACAAAAGTGTTTTCCAACGGCGCGGATCTGTCAGGCGTGACGGAAGAAGCTCCTCTTAAAC |
| | | TGAGTAAAGCAGTCCACAAAGCAGTACTCACTATTGATGAAAAGGGTACCGAGGCGGCCGGAGCTATGTTC |
| | | CTCGAAGCTATTCCTATGAGTATTCCCCCTGAAGTTAAATTTAATAAGCCTTTCGTGTTTCTCATGATAGA |
| | | GCAGAACACGAAAAGCCCTCTGTTTATGGGCAAGGTCGTCAACCCAACACAGAAGTAGCTGTGCCTTCTAG |
| | | TTGCCAGCCATCTGTTGTTTGCCCCTCCCCCGTGCCTTCCTTGACCCTGGAAGGTGCCACTCCCACTGTCC |
| | | TTTCCTAATAAAATGAGGAAATTGCATCGCATTGTCTGAGTAGGTGTCATTCTATTCTGGGGGGTGGGGGG |
| | | GGCAGGACAGCAAGGGGGAGGATTGGGAAGACAATAGCAGGCATGCTGGGGAtgggctcactatgctgccg |
| | | cccagtgggactttggaaatacaatgtgtcaactcttgacagggctctattttataggcttcttctctgga |
| | | atcttcttcatcatcctcctgacaatcgataggtacctggctgtcgtccatgctgtgtttgctttaaaagc |
| | | caggacggtcacctttggggtggtgacaagtgtgatcacttgggtggtggctgtgtttgcgtctctcccag |
| | | gaatcatctttaccagatctcaaaaagaaggtcttcattacacctgcagctctcattttccatacagtcag |
| | | tatcaattctggaagaatttccagacattaaagatagtcatcttggggctggtcctgccgctgcttgtcat |
| | | ggtcatctgctactcgggaatcctaaaaactctgcttcggtgtcgaaatgagaagaagaggcacagggctg |
| | | tgaggcttatcttcaccatcatgattgtttattttctcttctgggctccctacaa |
| 12 | 5′ homology | GCTTTCATGAATTCCCCCAACAGAGCCAAGCTCTCCATCTAGTGGACAGGGAAGCTAGCAGCAAACCTTCC |
| | arm- | CTTCACTACAAAACTTCATTGCTTGGCCAAAAAGAGAGTTAATTCAATGTAGACATCTATGTAGGCAATTA |
| | promoter- | AAAACCTATTGATGTATAAAACAGTTTGCATTCATGGAGGGCAACTAAATACATTCTAGGACTTTATAAAA |
| | therapeutic | GATCACTTTTTATTTATGCACAGGGTGGAACAAGATGGATTATCAAGTGTCAAGTCCAATCTATGACATCA |
| | protein- | ATTATTATACATCGGAGCCCTGCCAAAAAATCAATGTGAAGCAAATCGCAGCCCGCCTCCTGCCTCCGCTC |
| | noncleavable | TACTCACTGGTGTTCATCTTTGGTTTTGTGGGCAACATGCTGGTCATCCTCATCCTGATAAACTGCAAAAG |
| | linker-GPA- | GCTGAAGAGCATGACTGACATCTACCTGCTCAACCTGGCCATCTCTGACCTGTTTTTCCTTCTTACTGTCC |
| | pA- | CCTTCGAACCTCAGAACACTCAAATGATTTAAATTTCTCAAATACATTCATTTCACATATAGGAAGTCACT |
| | 3′ homology | TTCATTTGGACCACTGGGTCTTGACATTAGAAATGAGAAGGTCCATGGCTCCACAACAGCTACCTCAGCCT |
| | arm | GGCACGTGCCCTGGCCTCAGAGATTCACAGTCCAGTTCTTTGTCCAGTTGGGTGGCTCCTGTCTACCACCT |
| | | TACCATGCCCACTTAACTGATGCAAAGTTAATATCACAAGTAGCAACCTGTTCCTTGCAGTGAAAATTTTA |
| | | CTTACCACTTTCATAGCCCCAAGATATCCATGTATCTTTATTAACAGGCGCTTAACAACTTGCATCATTTA |
| | | AAATGCCTCCCCTGCCTATCAGCTGATGATGGCCGCAGGAAGGTGGGCCTGGAAGATAACAGCTAGCAGGC |
| | | TAAGGTCAGACACTGACACTTGCAGTTGTCTTTGGTAGTTTTTTTGCACTAACTTCAGGAACCAGCTCATG |
| | | ATCTCAGGATGTACGGGAAGATTATTTTCGTGTTGTTGCTCAGTGAGATCGTTTCTATCTCCGCTGAAGAC |
| | | CCTCAAGGCGACGCCGCTCAAAAGACGGACACGAGCCATCACGACCAAGACCATCCCACGTTTAATAAAAT |
| | | AACACCTAATCTCGCCGAATTTGCATTTTCACTGTATAGGCAACTCGCCCATCAAAGCAATTCTACAAACA |
| | | TTTTCTTTAGCCCGGTCAGTATAGCGACTGCTTTCGCCATGCTGTCTCTCGGTACAAAAGCCGATACCCAT |
| | | GACGAGATTTTGGAAGGACTCAACTTTAATCTGACCGAAATACCCGAAGCACAAATTCACGAGGGGTTTCA |
| | | AGAGCTGCTGCGAACTTTGAATCAACCCGATTCCCAACTGCAACTCACGACAGGTAACGGGTTGTTTCTGA |
| | | GTGAAGGGCTTAAACTGGTTGACAAATTCCTGGAAGACGTGAAGAAACTCTATCATAGTGAGGCATTTACA |
| | | GTTAATTTTGGAGATACCGAGGAAGCTAAGAAGCAAATTAATGACTATGTTGAGAAAGGCACGCAGGGAAA |
| | | GATCGTTGACCTCGTGAAAGAACTGGATCGAGATACGGTGTTCGCCCTCGTCAACTATATATTCTTCAAGG |
| | | GGAAGTGGGAACGCCCATTCGAGGTGAAAGATACAGAAGAGGAAGATTTTCATGTAGATCAAGTTACAACA |
| | | GTAAAAGTACCCATGATGAAAAGACTCGGGATGTTCAATATCCAACATTGCAAGAAGTTGTCATCTTGGGT |
| | | CCTTTTGATGAAGTATCTTGGGAACGCAACGGCTATATTCTTTCTCCCGGACGAAGGCAAGCTGCAACATC |
| | | TCGAGAACGAGCTGACTCATGACATTATTACGAAATTTCTTGAGAACGAGGATCGCCGGAGCGCGTCCCTG |
| | | CACCTGCCTAAGCTCAGCATAACAGGTACGTACGACCTCAAATCCGTGTTGGGACAGTTGGGGATTACGAA |
| | | AGTGTTTTCCAACGGAGCGGATCTGAGCGGTGTGACCGAAGAAGCTCCACTTAAACTGTCAAAAGCGGTCC |
| | | ACAAAGCCGTTTTGACTATAGATGAAAAGGGTACAGAGGCCGCCGGCGCGATGTTCCTGGAAGCTATCCCG |
| | | ATGTCCATACCACCAGAAGTGAAATTTAATAAGCCTTTCGTGTTTCTGATGATAGAGCAGAACACAAAATC |
| | | CCCACTGTTTATGGGCAAGGTCGTCAACCCAACACAGAAGGGTTCAGGCGGGTCCGGCGGAAGTGGGCTGA |
| | | TAATATTCGGCGTTATGGCCGGCGTGATCGGTACAATTCTGCTCATCAGTTATGGGATACGGAGATTGTGA |
| | | CTGTGCCTTCTAGTTGCCAGCCATCTGTTGTTTGCCCCTCCCCCGTGCCTTCCTTGACCCTGGAAGGTGCC |
| | | ACTCCCACTGTCCTTTCCTAATAAAATGAGGAAATTGCATCGCATTGTCTGAGTAGGTGTCATTCTATTCT |
| | | GGGGGGTGGGGTGGGGCAGGACAGCAAGGGGGAGGATTGGGAAGACAATAGCAGGCATGCTGGGGAtgggc |
| | | tcactatgctgccgcccagtgggactttggaaatacaatgtgtcaactcttgacagggctctattttatag |
| | | gcttcttctctggaatcttcttcatcatcctcctgacaatcgataggtacctggctgtcgtccatgctgtg |
| | | tttgctttaaaagccaggacggtcacctttggggtggtgacaagtgtgatcacttgggtggtggctgtgtt |
| | | tgcgtctctcccaggaatcatctttaccagatctcaaaaagaaggtcttcattacacctgcagctctcatt |
| | | ttccatacagtcagtatcaattctggaagaatttccagacattaaagatagtcatcttggggctggtcctg |
| | | ccgctgcttgtcatggtcatctgctactcgggaatcctaaaaactctgcttcggtgtcgaaatgagaagaa |
| | | gaggcacagggctgtgaggcttatcttcaccatcatgattgtttattttctcttctgggctccctacaa |
| 13 | 5′ homology | GCTTTCATGAATTCCCCCAACAGAGCCAAGCTCTCCATCTAGTGGACAGGGAAGCTAGCAGCAAACCTTCC |
| | arm- | CTTCACTACAAAACTTCATTGCTTGGCCAAAAAGAGAGTTAATTCAATGTAGACATCTATGTAGGCAATTA |
| | promoter- - | AAAACCTATTGATGTATAAAACAGTTTGCATTCATGGAGGGCAACTAAATACATTCTAGGACTTTATAAAA |
| | therapeutic | GATCACTTTTTATTTATGCACAGGGTGGAACAAGATGGATTATCAAGTGTCAAGTCCAATCTATGACATCA |
| | protein- | ATTATTATACATCGGAGCCCTGCCAAAAAATCAATGTGAAGCAAATCGCAGCCCGCCTCCTGCCTCCGCTC |
| | noncleavable | TACTCACTGGTGTTCATCTTTGGTTTTGTGGGCAACATGCTGGTCATCCTCATCCTGATAAACTGCAAAAG |
| | linker-GPA- | GCTGAAGAGCATGACTGACATCTACCTGCTCAACCTGGCCATCTCTGACCTGTTTTTCCTTCTTACTGTCC |
| | GPA(C-term)- | CCTTCGAACCTCAGAACACTCAAATGATTTAAATTTCTCAAACTGGGTCTTGACATTAGAAATGAGAAGGT |
| | pA- | CCATGGCTCCAATACATTCATTTCACATATAGGAAGTCACTTTCATTTGGACCCAACAGCTACCTCAGCCT |
| | 3′ homology | GGCACGTGCCCTGGCCTCAGAGATTCACAGTCCAGTTCTTTGTCCAGTTGGGTGGCTCCTGTCTACCACCT |
| | arm | TACCATGCCCACTTAACTGATGCAAAGTTAATATCACAAGTAGCAACCTGTTCCTTGCAGTGAAAATTTTA |
| | | CTTACCACTTTCATAGCCCCAAGATATCCATGTATCTTTATTAACAGGCGCTTAACAACTTGCATCATTTA |
| | | AAATGCCTCCCCTGCCTATCAGCTGATGATGGCCGCAGGAAGGTGGGCCTGGAAGATAACAGCTAGCAGGC |
| | | TAAGGTCAGACACTGACACTTGCAGTTGTCTTTGGTAGTTTTTTTGCACTAACTTCAGGAACCAGCTCATG |
| | | ATCTCAGGATGTACGGGAAGATTATTTTCGTGTTGTTGCTCAGTGAGATCGTTTCTATCTCCGCTGAAGAC |
| | | CCTCAAGGCGACGCCGCTCAAAAGACGGACACGAGCCATCACGACCAAGACCATCCCACGTTTAATAAAAT |
| | | AACACCTAATCTCGCCGAATTTGCATTTTCACTGTATAGGCAACTCGCCCATCAAAGCAATTCTACAAACA |
| | | TTTTCTTTAGCCCGGTCAGTATAGCGACTGCTTTCGCCATGCTGTCTCTCGGTACAAAAGCCGATACCCAT |
| | | GACGAGATTTTGGAAGGACTCAACTTTAATCTGACCGAAATACCCGAAGCACAAATTCACGAGGGGTTTCA |
| | | AGAGCTGCTGCGAACTTTGAATCAACCCGATTCCCAACTGCAACTCACGACAGGTAACGGGTTGTTTCTGA |
| | | GTGAAGGGCTTAAACTGGTTGACAAATTCCTGGAAGACGTGAAGAAACTCTATCATAGTGAGGCATTTACA |
| | | GTTAATTTTGGAGATACCGAGGAAGCTAAGAAGCAAATTAATGACTATGTTGAGAAAGGCACGCAGGGAAA |
| | | GATCGTTGACCTCGTGAAAGAACTGGATCGAGATACGGTGTTCGCCCTCGTCAACTATATATTCTTCAAGG |
| | | GGAAGTGGGAACGCCCATTCGAGGTGAAAGATACAGAAGAGGAAGATTTTCATGTAGATCAAGTTACAACA |
| | | GTAAAAGTACCCATGATGAAAAGACTCGGGATGTTCAATATCCAACATTGCAAGAAGTTGTCATCTTGGGT |
| | | CCTTTTGATGAAGTATCTTGGGAACGCAACGGCTATATTCTTTCTCCCGGACGAAGGCAAGCTGCAACATC |
| | | TCGAGAACGAGCTGACTCATGACATTATTACGAAATTTCTTGAGAACGAGGATCGCCGGAGCGCGTCCCTG |
| | | CACCTGCCTAAGCTCAGCATAACAGGTACGTACGACCTCAAATCCGTGTTGGGACAGTTGGGGATTACGAA |
| | | AGTGTTTTCCAACGGAGCGGATCTGAGCGGTGTGACCGAAGAAGCTCCACTTAAACTGTCAAAAGCGGTCC |
| | | ACAAAGCCGTTTTGACTATAGATGAAAAGGGTACAGAGGCCGCCGGCGCGATGTTCCTGGAAGCTATCCCG |
| | | ATGTCCATACCACCAGAAGTGAAATTTAATAAGCCTTTCGTGTTTCTGATGATAGAGCAGAACACAAAATC |
| | | CCCACTGTTTATGGGCAAGGTCGTCAACCCAACACAGAAGGGTTCAGGCGGGTCCGGCGGAAGTGGGCTGA |
| | | TAATATTCGGCGTTATGGCCGGCGTGATCGGTACAATTCTGCTCATCAGTTATGGGATACGGAGATTGATC |
| | | AAGAAGAGTCCTTCCGACGTCAAGCCACTGCCAAGCCCAGATACCGATGTTCCGCTTTCTTCAGTGGAGAT |
| | | TGAGAACCCCGAAACTTCTGACCAGTGACTGTGCCTTCTAGTTGCCAGCCATCTGTTGTTTGCCCCTCCCC |
| | | CGTGCCTTCCTTGACCCTGGAAGGTGCCACTCCCACTGTCCTTTCCTAATAAAATGAGGAAATTGCATCGC |
| | | ATTGTCTGAGTAGGTGTCATTCTATTCTGGGGGGTGGGGTGGGGCAGGACAGCAAGGGGGAGGATTGGGAA |
| | | GACAATAGCAGGCATGCTGGGGAtgggctcactatgctgccgcccagtgggactttggaaatacaatgtgt |
| | | caactcttgacagggctctattttataggcttcttctctggaatcttcttcatcatcctcctgacaatcga |
| | | taggtacctggctgtcgtccatgctgtgtttgctttaaaagccaggacggtcacctttggggtggtgacaa |
| | | gtgtgatcacttgggtggtggctgtgtttgcgtctctcccaggaatcatctttaccagatctcaaaaagaa |
| | | ggtcttcattacacctgcagctctcattttccatacagtcagtatcaattctggaagaatttccagacatt |
| | | aaagatagtcatcttggggctggtcctgccgctgcttgtcatggtcatctgctactcgggaatcctaaaaa |
| | | ctctgcttcggtgtcgaaatgagaagaagaggcacagggctgtgaggcttatcttcaccatcatgattgtt |
| | | tattttctcttctgggctccctacaa |
| 14 | 5′ homology | GCTTTCATGAATTCCCCCAACAGAGCCAAGCTCTCCATCTAGTGGACAGGGAAGCTAGCAGCAAACCTTCC |
| | arm- | CTTCACTACAAAACTTCATTGCTTGGCCAAAAAGAGAGTTAATTCAATGTAGACATCTATGTAGGCAATTA |
| | promoter- | AAAACCTATTGATGTATAAAACAGTTTGCATTCATGGAGGGCAACTAAATACATTCTAGGACTTTATAAAA |
| | therapeutic | GATCACTTTTTATTTATGCACAGGGTGGAACAAGATGGATTATCAAGTGTCAAGTCCAATCTATGACATCA |
| | protein- | ATTATTATACATCGGAGCCCTGCCAAAAAATCAATGTGAAGCAAATCGCAGCCCGCCTCCTGCCTCCGCTC |
| | cleavable | TACTCACTGGTGTTCATCTTTGGTTTTGTGGGCAACATGCTGGTCATCCTCATCCTGATAAACTGCAAAAG |
| | linker-GPA- | GCTGAAGAGCATGACTGACATCTACCTGCTCAACCTGGCCATCTCTGACCTGTTTTTCCTTCTTACTGTCC |
| | pA- | CCTTCGAACCTCAGAACACTCAAATGATTTAAATTTCTCAAATACATTCATTTCACATATAGGAAGTCACT |
| | 3′ homology | TTCATTTGGACCACTGGGTCTTGACATTAGAAATGAGAAGGTCCATGGCTCCACAACAGCTACCTCAGCCT |
| | arm | GGCACGTGCCCTGGCCTCAGAGATTCACAGTCCAGTTCTTTGTCCAGTTGGGTGGCTCCTGTCTACCACCT |
| | | TACCATGCCCACTTAACTGATGCAAAGTTAATATCACAAGTAGCAACCTGTTCCTTGCAGTGAAAATTTTA |
| | | CTTACCACTTTCATAGCCCCAAGATATCCATGTATCTTTATTAACAGGCGCTTAACAACTTGCATCATTTA |
| | | AAATGCCTCCCCTGCCTATCAGCTGATGATGGCCGCAGGAAGGTGGGCCTGGAAGATAACAGCTAGCAGGC |
| | | TAAGGTCAGACACTGACACTTGCAGTTGTCTTTGGTAGTTTTTTTGCACTAACTTCAGGAACCAGCTCATG |
| | | ATCTCAGGATGTACGGGAAGATTATTTTCGTGTTGTTGCTCAGTGAGATCGTTTCTATCTCCGCTGAAGAC |
| | | CCTCAAGGCGACGCCGCTCAAAAGACGGACACGAGCCATCACGACCAAGACCATCCCACGTTTAATAAAAT |
| | | AACACCTAATCTCGCCGAATTTGCATTTTCACTGTATAGGCAACTCGCCCATCAAAGCAATTCTACAAACA |
| | | TTTTCTTTAGCCCGGTCAGTATAGCGACTGCTTTCGCCATGCTGTCTCTCGGTACAAAAGCCGATACCCAT |
| | | GACGAGATTTTGGAAGGACTCAACTTTAATCTGACCGAAATACCCGAAGCACAAATTCACGAGGGGTTTCA |
| | | AGAGCTGCTGCGAACTTTGAATCAACCCGATTCCCAACTGCAACTCACGACAGGTAACGGGTTGTTTCTGA |
| | | GTGAAGGGCTTAAACTGGTTGACAAATTCCTGGAAGACGTGAAGAAACTCTATCATAGTGAGGCATTTACA |
| | | GTTAATTTTGGAGATACCGAGGAAGCTAAGAAGCAAATTAATGACTATGTTGAGAAAGGCACGCAGGGAAA |
| | | GATCGTTGACCTCGTGAAAGAACTGGATCGAGATACGGTGTTCGCCCTCGTCAACTATATATTCTTCAAGG |
| | | GGAAGTGGGAACGCCCATTCGAGGTGAAAGATACAGAAGAGGAAGATTTTCATGTAGATCAAGTTACAACA |
| | | GTAAAAGTACCCATGATGAAAAGACTCGGGATGTTCAATATCCAACATTGCAAGAAGTTGTCATCTTGGGT |
| | | CCTTTTGATGAAGTATCTTGGGAACGCAACGGCTATATTCTTTCTCCCGGACGAAGGCAAGCTGCAACATC |
| | | TCGAGAACGAGCTGACTCATGACATTATTACGAAATTTCTTGAGAACGAGGATCGCCGGAGCGCGTCCCTG |
| | | CACCTGCCTAAGCTCAGCATAACAGGTACGTACGACCTCAAATCCGTGTTGGGACAGTTGGGGATTACGAA |
| | | AGTGTTTTCCAACGGAGCGGATCTGAGCGGTGTGACCGAAGAAGCTCCACTTAAACTGTCAAAAGCGGTCC |
| | | ACAAAGCCGTTTTGACTATAGATGAAAAGGGTACAGAGGCCGCCGGCGCGATGTTCCTGGAAGCTATCCCG |
| | | ATGTCCATACCACCAGAAGTGAAATTTAATAAGCCTTTCGTGTTTCTGATGATAGAGCAGAACACAAAATC |
| | | CCCACTGTTTATGGGCAAGGTCGTCAACCCAACACAGAAGGGTTCAGGCGGGTCCGGCGGAAGTGGGCCAC |
| | | TTGGTATGTGGTCTAGGCTGATAATATTCGGCGTTATGGCCGGCGTGATCGGTACAATTCTGCTCATCAGT |
| | | TATGGGATACGGAGATTGTGACTGTGCCTTCTAGTTGCCAGCCATCTGTTGTTTGCCCCTCCCCCGTGCCT |
| | | TCCTTGACCCTGGAAGGTGCCACTCCCACTGTCCTTTCCTAATAAAATGAGGAAATTGCATCGCATTGTCT |
| | | GAGTAGGTGTCATTCTATTCTGGGGGGTGGGGTGGGGCAGGACAGCAAGGGGGAGGATTGGGAAGACAATA |
| | | GCAGGCATGCTGGGGAtgggctcactatgctgccgcccagtgggactttggaaatacaatgtgtcaactct |
| | | tgacagggctctattttataggcttcttctctggaatcttcttcatcatcctcctgacaatcgataggtac |
| | | ctggctgtcgtccatgctgtgtttgctttaaaagccaggacggtcacctttggggtggtgacaagtgtgat |
| | | cacttgggtggtggctgtgtttgcgtctctcccaggaatcatctttaccagatctcaaaaagaaggtcttc |
| | | attacacctgcagctctcattttccatacagtcagtatcaattctggaagaatttccagacattaaagata |
| | | gtcatcttggggctggtcctgccgctgcttgtcatggtcatctgctactcgggaatcctaaaaactctgct |
| | | tcggtgtcgaaatgagaagaagaggcacagggctgtgaggcttatcttcaccatcatgattgtttattttc |
| | | tcttctgggctccctacaa |
| 15 | 5′ homology | GCTTTCATGAATTCCCCCAACAGAGCCAAGCTCTCCATCTAGTGGACAGGGAAGCTAGCAGCAAACCTTCC |
| | arm- | CTTCACTACAAAACTTCATTGCTTGGCCAAAAAGAGAGTTAATTCAATGTAGACATCTATGTAGGCAATTA |
| | promoter- | AAAACCTATTGATGTATAAAACAGTTTGCATTCATGGAGGGCAACTAAATACATTCTAGGACTTTATAAAA |
| | therapeutic | GATCACTTTTTATTTATGCACAGGGTGGAACAAGATGGATTATCAAGTGTCAAGTCCAATCTATGACATCA |
| | protein- | ATTATTATACATCGGAGCCCTGCCAAAAAATCAATGTGAAGCAAATCGCAGCCCGCCTCCTGCCTCCGCTC |
| | cleavable | TACTCACTGGTGTTCATCTTTGGTTTTGTGGGCAACATGCTGGTCATCCTCATCCTGATAAACTGCAAAAG |
| | linker-GPA- | GCTGAAGAGCATGACTGACATCTACCTGCTCAACCTGGCCATCTCTGACCTGTTTTTCCTTCTTACTGTCC |
| | GPA(C-term)- | CCTTCGAACCTCAGAACACTCAAATGATTTAAATTTCTCAAATACATTCATTTCACATATAGGAAGTCACT |
| | pA- | TTCATTTGGACCACTGGGTCTTGACATTAGAAATGAGAAGGTCCATGGCTCCACAACAGCTACCTCAGCCT |
| | 3′ homology | GGCACGTGCCCTGGCCTCAGAGATTCACAGTCCAGTTCTTTGTCCAGTTGGGTGGCTCCTGTCTACCACCT |
| | arm | TACCATGCCCACTTAACTGATGCAAAGTTAATATCACAAGTAGCAACCTGTTCCTTGCAGTGAAAATTTTA |
| | | CTTACCACTTTCATAGCCCCAAGATATCCATGTATCTTTATTAACAGGCGCTTAACAACTTGCATCATTTA |
| | | AAATGCCTCCCCTGCCTATCAGCTGATGATGGCCGCAGGAAGGTGGGCCTGGAAGATAACAGCTAGCAGGC |
| | | TAAGGTCAGACACTGACACTTGCAGTTGTCTTTGGTAGTTTTTTTGCACTAACTTCAGGAACCAGCTCATG |
| | | ATCTCAGGATGTACGGGAAGATTATTTTCGTGTTGTTGCTCAGTGAGATCGTTTCTATCTCCGCTGAAGAC |
| | | CCTCAAGGCGACGCCGCTCAAAAGACGGACACGAGCCATCACGACCAAGACCATCCCACGTTTAATAAAAT |
| | | AACACCTAATCTCGCCGAATTTGCATTTTCACTGTATAGGCAACTCGCCCATCAAAGCAATTCTACAAACA |
| | | TTTTCTTTAGCCCGGTCAGTATAGCGACTGCTTTCGCCATGCTGTCTCTCGGTACAAAAGCCGATACCCAT |
| | | GACGAGATTTTGGAAGGACTCAACTTTAATCTGACCGAAATACCCGAAGCACAAATTCACGAGGGGTTTCA |
| | | AGAGCTGCTGCGAACTTTGAATCAACCCGATTCCCAACTGCAACTCACGACAGGTAACGGGTTGTTTCTGA |
| | | GTGAAGGGCTTAAACTGGTTGACAAATTCCTGGAAGACGTGAAGAAACTCTATCATAGTGAGGCATTTACA |
| | | GTTAATTTTGGAGATACCGAGGAAGCTAAGAAGCAAATTAATGACTATGTTGAGAAAGGCACGCAGGGAAA |
| | | GATCGTTGACCTCGTGAAAGAACTGGATCGAGATACGGTGTTCGCCCTCGTCAACTATATATTCTTCAAGG |
| | | GGAAGTGGGAACGCCCATTCGAGGTGAAAGATACAGAAGAGGAAGATTTTCATGTAGATCAAGTTACAACA |
| | | GTAAAAGTACCCATGATGAAAAGACTCGGGATGTTCAATATCCAACATTGCAAGAAGTTGTCATCTTGGGT |
| | | CCTTTTGATGAAGTATCTTGGGAACGCAACGGCTATATTCTTTCTCCCGGACGAAGGCAAGCTGCAACATC |
| | | TCGAGAACGAGCTGACTCATGACATTATTACGAAATTTCTTGAGAACGAGGATCGCCGGAGCGCGTCCCTG |
| | | CACCTGCCTAAGCTCAGCATAACAGGTACGTACGACCTCAAATCCGTGTTGGGACAGTTGGGGATTACGAA |
| | | AGTGTTTTCCAACGGAGCGGATCTGAGCGGTGTGACCGAAGAAGCTCCACTTAAACTGTCAAAAGCGGTCC |
| | | ACAAAGCCGTTTTGACTATAGATGAAAAGGGTACAGAGGCCGCCGGCGCGATGTTCCTGGAAGCTATCCCG |
| | | ATGTCCATACCACCAGAAGTGAAATTTAATAAGCCTTTCGTGTTTCTGATGATAGAGCAGAACACAAAATC |
| | | CCCACTGTTTATGGGCAAGGTCGTCAACCCAACACAGAAGGGTTCAGGCGGGTCCGGCGGAAGTGGGCCAC |
| | | TTGGTATGTGGTCTAGGCTGATAATATTCGGCGTTATGGCCGGCGTGATCGGTACAATTCTGCTCATCAGT |
| | | TATGGGATACGGAGATTGATCAAGAAGAGTCCTTCCGACGTCAAGCCACTGCCAAGCCCAGATACCGATGT |
| | | TCCGCTTTCTTCAGTGGAGATTGAGAACCCCGAAACTTCTGACCAGTGACTGTGCCTTCTAGTTGCCAGCC |
| | | ATCTGTTGTTTGCCCCTCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGAtgggctcactatgctgccgcccagtgg |
| | | gactttggaaatacaatgtgtcaactcttgacagggctctattttataggcttcttctctggaatcttctt |
| | | catcatcctcctgacaatcgataggtacctggctgtcgtccatgctgtgtttgctttaaaagccaggacgg |
| | | tcacctttggggtggtgacaagtgtgatcacttgggtggtggctgtgtttgcgtctctcccaggaatcatc |
| | | tttaccagatctcaaaaagaaggtcttcattacacctgcagctctcattttccatacagtcagtatcaatt |
| | | ctggaagaatttccagacattaaagatagtcatcttggggctggtcctgccgctgcttgtcatggtcatct |
| | | gctactcgggaatcctaaaaactctgcttcggtgtcgaaatgagaagaagaggcacagggctgtgaggctt |
| | | atcttcaccatcatgattgtttattttctcttctgggctccctacaa |
| 16 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | 3′ homology | gaggtgtagcgcaggcggcggctgcgggcctgggccctcggccccactgaccctcttctctgcacagCTCC |
| | arm | TAAGCCACTGCCTGCTGGTGACCCTGGCCGCCCACCTCCCCGCCGAGTTCACCCCTGCGGTGCACGCCTCC |
| | | CTGGACAAGTTCCTGGCTTCTGTGAGCACCGTGCTGACCTCCAAATACCGTGAGGGCAGGGGAAGTCTTCT |
| | | AACATGCGGGGACGTGGAGGAAAATCCCGGCCCCATGCCTTCATCAGTATCTTGGGGAATACTGCTCCTTG |
| | | CTGGGTTGTGTTGTCTCGTACCCGTGAGTCTCGCCGAAGACCCTCAAGGCGACGCCGCACAAAAGACTGAC |
| | | ACTTCTCATCACGACCAAGACCATCCTACATTTAATAAAATTACTCCAAATCTCGCCGAATTTGCGTTTTC |
| | | TCTGTATAGGCAACTCGCTCACCAATCTAATTCAACGAACATATTCTTTTCACCTGTTTCCATAGCCACCG |
| | | CTTTCGCCATGCTGAGTTTGGGAACAAAAGCAGATACCCATGACGAGATACTCGAAGGACTCAACTTTAAT |
| | | CTGACAGAAATCCCTGAAGCACAAATTCACGAGGGTTTTCAAGAGCTGCTGAGAACTTTGAATCAACCCGA |
| | | TTCCCAATTGCAACTCACAACAGGAAACGGTTTGTTTCTTTCAGAAGGGCTCAAACTGGTCGACAAATTCC |
| | | TCGAAGACGTGAAGAAACTTTATCATAGCGAGGCTTTTACCGTGAATTTTGGAGATACGGAAGAAGCTAAG |
| | | AAGCAAATAAATGACTATGTCGAAAAGGGGACACAGGGAAAGATAGTTGACCTGGTGAAAGAACTGGATAG |
| | | GGATACTGTGTTCGCGCTCGTCAACTATATCTTCTTCAAGGGGAAGTGGGAACGGCCATTCGAGGTTAAAG |
| | | ATACAGAAGAGGAAGATTTTCATGTAGATCAAGTCACAACAGTCAAAGTTCCAATGATGAAACGCCTCGGG |
| | | ATGTTCAATATACAACATTGCAAGAAACTTAGCTCATGGGTCCTTTTGATGAAGTATCTCGGGAACGCTAC |
| | | AGCGATATTCTTTCTCCCAGACGAAGGTAAGCTGCAACATCTTGAGAACGAGCTGACACATGACATAATAA |
| | | CAAAATTTCTTGAGAACGAGGATCGCCGGTCCGCATCCCTGCACCTGCCGAAGCTTAGCATAACCGGCACA |
| | | TACGACTTGAAATCTGTTCTTGGGCAGCTTGGTATTACAAAAGTGTTTTCCAACGGCGCGGATCTGTCAGG |
| | | CGTGACGGAAGAAGCTCCTCTTAAACTGAGTAAAGCAGTCCACAAAGCAGTACTCACTATTGATGAAAAGG |
| | | GTACCGAGGCGGCCGGAGCTATGTTCCTCGAAGCTATTCCTATGAGTATTCCCCCTGAAGTTAAATTTAAT |
| | | AAGCCTTTCGTGTTTCTCATGATAGAGCAGAACACGAAAAGCCCTCTGTTTATGGGCAAGGTCGTCAACCC |
| | | AACACAGAAGTAGtggccatgcttcttgccccttgggcctccccccagcccctcctccccttcctgcaccc |
| | | gtacccccgtggtctttgaataaagtctgagtgggcggcagcctgtgtgtgcctgagttttttccctcagc |
| | | aaacgtgccaggcatgggcgtggacagcagctgggacacacatggctagaacctctctgcagctggatagg |
| | | gtaggaaaaggcaggggcgggaggaggggatggaggagggaaagtggagccaccgcgaagtccagctggaa |
| | | aaacgctggaccctagagtgctttgaggatgcatttgctctttcccgagttttattcccagacttttcaga |
| | | ttcaatgcaggtttgctgaaataatgaatttatccatctttacgtttctgggcactctgtgccaagaactg |
| | | gctggctttctgcctgggacgtcactggtttcccagaggtcctcccacatatggggggggtaggtcagaga |
| | | agtcccactccagcatggctgcattgatcccccatcgttcccactagtctccgtaaaacctcccagataca |
| | | ggcacagtctagatgaaatcaggggtgcggggtgcaactgcaggccccaggcaattcaataggggctctac |
| | | tttcacccccaggtcaccccagaatgctcacacaccagacactgacgccctggggctgtcaagatcaggcg |
| | | tttgtctctgggcccagctcagggcccagctcagcacccactcagctcccctgaggctggggagcctgtcc |
| | | cattgcgactggagaggagagcggggccacagaggcctggctagaaggtcccttctccctggtgtgtgttt |
| | | tctctctgctgagcaggcttgcagtgcctggggtatca |
| 17 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCcggcgggccgggagcgatctgggtcgaggggcgagatggcgccttcctc |
| | protein-pA- | gcagggcagaggatACGCGCACAAGCTTCGGGTGGACCCGGTCAACTTCAAGgtgagcacgcgggttgcgg |
| | 3′ homology | gaggtgtagcgcaggcggcggctgcgggcctgggccctcggccccactgaccctcttctctgcacagCTCC |
| | arm | TAAGCCACTGCCTGCTGGTGACCCTGGCCGCCCACCTCCCCGCCGAGTTCACCCCTGCGGTGCACGCCTCC |
| | | CTGGACAAGTTCCTGGCTTCTGTGAGCACCGTGCTGACCTCCAAATACCGTGAGGGCAGGGGAAGTCTTCT |
| | | AACATGCGGGGACGTGGAGGAAAATCCCGGCCCCATGCCTTCATCAGTATCTTGGGGAATACTGCTCCTTG |
| | | CTGGGTTGTGTTGTCTCGTACCCGTGAGTCTCGCCGAAGACCCTCAAGGCGACGCCGCACAAAAGACTGAC |
| | | ACTTCTCATCACGACCAAGACCATCCTACATTTAATAAAATTACTCCAAATCTCGCCGAATTTGCGTTTTC |
| | | TCTGTATAGGCAACTCGCTCACCAATCTAATTCAACGAACATATTCTTTTCACCTGTTTCCATAGCCACCG |
| | | CTTTCGCCATGCTGAGTTTGGGAACAAAAGCAGATACCCATGACGAGATACTCGAAGGACTCAACTTTAAT |
| | | CTGACAGAAATCCCTGAAGCACAAATTCACGAGGGTTTTCAAGAGCTGCTGAGAACTTTGAATCAACCCGA |
| | | TTCCCAATTGCAACTCACAACAGGAAACGGTTTGTTTCTTTCAGAAGGGCTCAAACTGGTCGACAAATTCC |
| | | TCGAAGACGTGAAGAAACTTTATCATAGCGAGGCTTTTACCGTGAATTTTGGAGATACGGAAGAAGCTAAG |
| | | AAGCAAATAAATGACTATGTCGAAAAGGGGACACAGGGAAAGATAGTTGACCTGGTGAAAGAACTGGATAG |
| | | GGATACTGTGTTCGCGCTCGTCAACTATATCTTCTTCAAGGGGAAGTGGGAACGGCCATTCGAGGTTAAAG |
| | | ATACAGAAGAGGAAGATTTTCATGTAGATCAAGTCACAACAGTCAAAGTTCCAATGATGAAACGCCTCGGG |
| | | ATGTTCAATATACAACATTGCAAGAAACTTAGCTCATGGGTCCTTTTGATGAAGTATCTCGGGAACGCTAC |
| | | AGCGATATTCTTTCTCCCAGACGAAGGTAAGCTGCAACATCTTGAGAACGAGCTGACACATGACATAATAA |
| | | CAAAATTTCTTGAGAACGAGGATCGCCGGTCCGCATCCCTGCACCTGCCGAAGCTTAGCATAACCGGCACA |
| | | TACGACTTGAAATCTGTTCTTGGGCAGCTTGGTATTACAAAAGTGTTTTCCAACGGCGCGGATCTGTCAGG |
| | | CGTGACGGAAGAAGCTCCTCTTAAACTGAGTAAAGCAGTCCACAAAGCAGTACTCACTATTGATGAAAAGG |
| | | GTACCGAGGCGGCCGGAGCTATGTTCCTCGAAGCTATTCCTATGAGTATTCCCCCTGAAGTTAAATTTAAT |
| | | AAGCCTTTCGTGTTTCTCATGATAGAGCAGAACACGAAAAGCCCTCTGTTTATGGGCAAGGTCGTCAACCC |
| | | AACACAGAAGTAGCTGTGCCTTCTAGTTGCCAGCCATCTGTTGTTTGCCCCTCCCCCGTGCCTTCCTTGAC |
| | | CCTGGAAGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGACAATAGCAGGCAT |
| | | GCTGGGGAtggccatgcttcttgccccttgggcctccccccagcccctectccccttcctgcacccgtacc |
| | | cccgtggtctttgaataaagtctgagtgggggcagcctgtgtgtgcctgagttttttccctcagcaaacgt |
| | | gccaggcatgggcgtggacagcagctgggacacacatggctagaacctctctgcagctggatagggtagga |
| | | aaaggcaggggcgggaggaggggatggaggagggaaagtggagccaccgcgaagtccagctggaaaaacgc |
| | | tggaccctagagtgctttgaggatgcatttgctctttcccgagttttattcccagacttttcagattcaat |
| | | gcaggtttgctgaaataatgaatttatccatctttacgtttctgggcactctgtgccaagaactggctggc |
| | | tttctgcctgggacgtcactggtttcccagaggtcctcccacatatgggtggtgggtaggtcagagaagtc |
| | | ccactccagcatggctgcattgatcccccatcgttcccactagtctccgtaaaacctcccagatacaggca |
| | | cagtctagatgaaatcaggggtgcggggtgcaactgcaggccccaggcaattcaataggggctctactttc |
| | | acccccaggtcaccccagaatgctcacacaccagacactgacgccctggggctgtcaagatcaggcgtttg |
| | | tctctgggcccagctcagggcccagctcagcacccactcagctcccctgaggctggggagcctgtcccatt |
| | | gcgactggagaggagagcggggccacagaggcctggctagaaggtcccttctccctggtgtgtgttttctc |
| | | tctgctgagcaggcttgcagtgcctggggtatca |
| 18 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | noncleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | 3′ homology | CTGGACAAGTTCCTGGCTTCTGTGAGCACCGTGCTGACCTCCAAATACCGTGAGGGCAGGGGAAGTCTTCT |
| | arm | AACATGCGGGGACGTGGAGGAAAATCCCGGCCCCATGTACGGGAAGATTATTTTCGTGTTGTTGCTCAGTG |
| | | AGATCGTTTCTATCTCCGCTGAAGACCCTCAAGGCGACGCCGCTCAAAAGACGGACACGAGCCATCACGAC |
| | | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCTGATAATATTCGGCGTTATGGCCGGCGTGATCGGTACAATTCTGCTCA |
| | | TCAGTTATGGGATACGGAGATTGTGAtggccatgcttcttgccccttgggcctccccccagcccctectcc |
| | | ccttcctgcacccgtacccccgtggtctttgaataaagtctgagtgggcggcagcctgtgtgtgcctgagt |
| | | tttttccctcagcaaacgtgccaggcatgggcgtggacagcagctgggacacacatggctagaacctctct |
| | | gcagctggatagggtaggaaaaggcaggggcgggaggaggggatggaggagggaaagtggagccaccgcga |
| | | agtccagctggaaaaacgctggaccctagagtgctttgaggatgcatttgctctttcccgagttttattcc |
| | | cagacttttcagattcaatgcaggtttgctgaaataatgaatttatccatctttacgtttctgggcactct |
| | | gtgccaagaactggctggctttctgcctgggacgtcactggtttcccagaggtcctcccacatatgggggg |
| | | ggtaggtcagagaagtcccactccagcatggctgcattgatcccccatcgttcccactagtctccgtaaaa |
| | | cctcccagatacaggcacagtctagatgaaatcaggggtgcggggtgcaactgcaggccccaggcaattca |
| | | ataggggctctactttcacccccaggtcaccccagaatgctcacacaccagacactgacgccctggggctg |
| | | tcaagatcaggcgtttgtctctgggcccagctcagggcccagctcagcacccactcagctcccctgaggct |
| | | ggggagcctgtcccattgcgactggagaggagagcggggccacagaggcctggctagaaggtcccttctcc |
| | | ctggtgtgtgttttctctctgctgagcaggcttgcagtgcctggggtatca |
| 19 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCcggcgggccgggagcgatctgggtcgaggggcgagatggcgccttcctc |
| | protein- | gcagggcagaggatACGCGCACAAGCTTCGGGTGGACCCGGTCAACTTCAAGgtgagcacgcgggttgcgg |
| | noncleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | pA- | CTGGACAAGTTCCTGGCTTCTGTGAGCACCGTGCTGACCTCCAAATACCGTGAGGGCAGGGGAAGTCTTCT |
| | 3′ homology | AACATGCGGGGACGTGGAGGAAAATCCCGGCCCCATGTACGGGAAGATTATTTTCGTGTTGTTGCTCAGTG |
| | arm | AGATCGTTTCTATCTCCGCTGAAGACCCTCAAGGCGACGCCGCTCAAAAGACGGACACGAGCCATCACGAC |
| | | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCTGATAATATTCGGCGTTATGGCCGGCGTGATCGGTACAATTCTGCTCA |
| | | TCAGTTATGGGATACGGAGATTGTGACTGTGCCTTCTAGTTGCCAGCCATCTGTTGTTTGCCCCTCCCCCG |
| | | TGCCTTCCTTGACCCTGGAAGGTGCCACTCCCACTGTCCTTTCCTAATAAAATGAGGAAATTGCATCGCAT |
| | | TGTCTGAGTAGGTGTCATTCTATTCTGGGGGGTGGGGTGGGGCAGGACAGCAAGGGGGAGGATTGGGAAGA |
| | | CAATAGCAGGCATGCTGGGGAtggccatgcttcttgccccttgggcctccccccagcccctectccccttc |
| | | ctgcacccgtacccccgtggtctttgaataaagtctgagtgggcggcagcctgtgtgtgcctgagtttttt |
| | | ccctcagcaaacgtgccaggcatgggcgtggacagcagctgggacacacatggctagaacctctctgcagc |
| | | tggatagggtaggaaaaggcaggggcgggaggaggggatggaggagggaaagtggagccaccgcgaagtcc |
| | | agctggaaaaacgctggaccctagagtgctttgaggatgcatttgctctttcccgagttttattcccagac |
| | | ttttcagattcaatgcaggtttgctgaaataatgaatttatccatctttacgtttctgggcactctgtgcc |
| | | aagaactggctggctttctgcctgggacgtcactggtttcccagaggtcctcccacatatgggtggtgggt |
| | | aggtcagagaagtcccactccagcatggctgcattgatcccccatcgttcccactagtctccgtaaaacct |
| | | cccagatacaggcacagtctagatgaaatcaggggtgcggggtgcaactgcaggccccaggcaattcaata |
| | | ggggctctactttcacccccaggtcaccccagaatgctcacacaccagacactgacgccctggggctgtca |
| | | agatcaggcgtttgtctctgggcccagctcagggcccagctcagcacccactcagctcccctgaggctggg |
| | | gagcctgtcccattgcgactggagaggagagcggggccacagaggcctggctagaaggtcccttctccctg |
| | | gtgtgtgttttctctctgctgagcaggcttgcagtgcctggggtatca |
| 20 | 5′ homology | GTTCCTGTCCTTCCCCACCACCAAGACCTACTTCCCGCACTTCGACCTGAGCCACGGCTCTGCCCAGGTTA |
| | arm-2A- - | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | noncleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | GPA(C-term)- | CTGGACAAGTTCCTGGCTTCTGTGAGCACCGTGCTGACCTCCAAATACCGTGAGGGCAGGGGAAGTCTTCT |
| | 3′ homology | AACATGCGGGGACGTGGAGGAAAATCCCGGCCCCATGTACGGGAAGATTATTTTCGTGTTGTTGCTCAGTG |
| | arm | AGATCGTTTCTATCTCCGCTGAAGACCCTCAAGGCGACGCCGCTCAAAAGACGGACACGAGCCATCACGAC |
| | | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCTGATAATATTCGGCGTTATGGCCGGCGTGATCGGTACAATTCTGCTCA |
| | | TCAGTTATGGGATACGGAGATTGATCAAGAAGAGTCCTTCCGACGTCAAGCCACTGCCAAGCCCAGATACC |
| | | GATGTTCCGCTTTCTTCAGTGGAGATTGAGAACCCCGAAACTTCTGACCAGTGAtggccatgcttcttgcc |
| | | ccttgggcctccccccagcccctcctccccttcctgcaccegtacccccgtggtctttgaataaagtctga |
| | | gtgggcggcagcctgtgtgtgcctgagttttttccctcagcaaacgtgccaggcatgggcgtggacagcag |
| | | ctgggacacacatggctagaacctctctgcagctggatagggtaggaaaaggcaggggcgggaggagggga |
| | | tggaggagggaaagtggagccaccgcgaagtccagctggaaaaacgctggaccctagagtgctttgaggat |
| | | gcatttgctctttcccgagttttattcccagacttttcagattcaatgcaggtttgctgaaataatgaatt |
| | | tatccatctttacgtttctgggcactctgtgccaagaactggctggctttctgcctgggacgtcactggtt |
| | | tcccagaggtcctcccacatatggggggggtaggtcagagaagtcccactccagcatggctgcattgatcc |
| | | cccatcgttcccactagtctccgtaaaacctcccagatacaggcacagtctagatgaaatcaggggtgcgg |
| | | ggtgcaactgcaggccccaggcaattcaataggggctctactttcacccccaggtcaccccagaatgctca |
| | | cacaccagacactgacgccctggggctgtcaagatcaggcgtttgtctctgggcccagctcagggcccagc |
| | | tcagcacccactcagctcccctgaggctggggagcctgtcccattgcgactggagaggagagcggggccac |
| | | agaggcctggctagaaggtcccttctccctggtgtgtgttttctctctgctgagcaggcttgcagtgcctg |
| | | gggtatca |
| 21 | 5′ homology | GTTCCTGTCCTTCCCCACCACCAAGACCTACTTCCCGCACTTCGACCTGAGCCACGGCTCTGCCCAGGTTA |
| | arm-2A- - | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | noncleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | GPA(C-term)- | CTGGACAAGTTCCTGGCTTCTGTGAGCACCGTGCTGACCTCCAAATACCGTGAGGGCAGGGGAAGTCTTCT |
| | pA- | AACATGCGGGGACGTGGAGGAAAATCCCGGCCCCATGTACGGGAAGATTATTTTCGTGTTGTTGCTCAGTG |
| | 3′ homology | AGATCGTTTCTATCTCCGCTGAAGACCCTCAAGGCGACGCCGCTCAAAAGACGGACACGAGCCATCACGAC |
| | arm | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCTGATAATATTCGGCGTTATGGCCGGCGTGATCGGTACAATTCTGCTCA |
| | | TCAGTTATGGGATACGGAGATTGATCAAGAAGAGTCCTTCCGACGTCAAGCCACTGCCAAGCCCAGATACC |
| | | GATGTTCCGCTTTCTTCAGTGGAGATTGAGAACCCCGAAACTTCTGACCAGTGACTGTGCCTTCTAGTTGC |
| | | CAGCCATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTGGGGC |
| | | AGGACAGCAAGGGGGAGGATTGGGAAGACAATAGCAGGCATGCTGGGGAtggccatgcttcttgccccttg |
| | | ggcctccccccagcccctcctccccttcctgcacccgtacccccgtggtctttgaataaagtctgagtggg |
| | | cggcagcctgtgtgtgcctgagttttttccctcagcaaacgtgccaggcatgggcgtggacagcagctggg |
| | | acacacatggctagaacctctctgcagctggatagggtaggaaaaggcaggggcgggaggaggggatggag |
| | | gagggaaagtggagccaccgcgaagtccagctggaaaaacgctggaccctagagtgctttgaggatgcatt |
| | | tgctctttcccgagttttattcccagacttttcagattcaatgcaggtttgctgaaataatgaatttatcc |
| | | atctttacgtttctgggcactctgtgccaagaactggctggctttctgcctgggacgtcactggtttccca |
| | | gaggtcctcccacatatgggtggtgggtaggtcagagaagtcccactccagcatggctgcattgatccccc |
| | | atcgttcccactagtctccgtaaaacctcccagatacaggcacagtctagatgaaatcaggggtgcggggt |
| | | gcaactgcaggccccaggcaattcaataggggctctactttcacccccaggtcaccccagaatgctcacac |
| | | accagacactgacgccctggggctgtcaagatcaggcgtttgtctctgggcccagctcagggcccagctca |
| | | gcacccactcagctcccctgaggctggggagcctgtcccattgcgactggagaggagagcggggccacaga |
| | | ggcctggctagaaggtcccttctccctggtgtgtgttttctctctgctgagcaggcttgcagtgcctgggg |
| | | tatca |
| 22 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | cleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | 3′ homology | CTGGACAAGTTCCTGGCTTCTGTGAGCACCGTGCTGACCTCCAAATACCGTGAGGGCAGGGGAAGTCTTCT |
| | arm | AACATGCGGGGACGTGGAGGAAAATCCCGGCCCCATGTACGGGAAGATTATTTTCGTGTTGTTGCTCAGTG |
| | | AGATCGTTTCTATCTCCGCTGAAGACCCTCAAGGCGACGCCGCTCAAAAGACGGACACGAGCCATCACGAC |
| | | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCCACTTGGTATGTGGTCTAGGCTGATAATATTCGGCGTTATGGCCGGCG |
| | | TGATCGGTACAATTCTGCTCATCAGTTATGGGATACGGAGATTGTGAtggccatgcttcttgccccttggg |
| | | cctccccccagcccctectccccttcctgcaccegtacccccgtggtctttgaataaagtctgagtgggcg |
| | | gcagcctgtgtgtgcctgagttttttccctcagcaaacgtgccaggcatgggcgtggacagcagctgggac |
| | | acacatggctagaacctctctgcagctggatagggtaggaaaaggcaggggcgggaggaggggatggagga |
| | | gggaaagtggagccaccgcgaagtccagctggaaaaacgctggaccctagagtgctttgaggatgcatttg |
| | | ctctttcccgagttttattcccagacttttcagattcaatgcaggtttgctgaaataatgaatttatccat |
| | | ctttacgtttctgggcactctgtgccaagaactggctggctttctgcctgggacgtcactggtttcccaga |
| | | ggtcctcccacatatgggtggtgggtaggtcagagaagtcccactccagcatggctgcattgatcccccat |
| | | cgttcccactagtctccgtaaaacctcccagatacaggcacagtctagatgaaatcaggggtgcggggtgc |
| | | aactgcaggccccaggcaattcaataggggctctactttcacccccaggtcaccccagaatgctcacacac |
| | | cagacactgacgccctggggctgtcaagatcaggcgtttgtctctgggcccagctcagggcccagctcagc |
| | | acccactcagctcccctgaggctggggagcctgtcccattgcgactggagaggagagcggggccacagagg |
| | | cctggctagaaggtcccttctccctggtgtgtgttttctctctgctgagcaggcttgcagtgcctggggta |
| | | tca |
| 23 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | cleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | pA- | CTGGACAAGTTCCTGGCTTCTGTGAGCACCGTGCTGACCTCCAAATACCGTGAGGGCAGGGGAAGTCTTCT |
| | 3′ homology | AACATGCGGGGACGTGGAGGAAAATCCCGGCCCCATGTACGGGAAGATTATTTTCGTGTTGTTGCTCAGTG |
| | arm | AGATCGTTTCTATCTCCGCTGAAGACCCTCAAGGCGACGCCGCTCAAAAGACGGACACGAGCCATCACGAC |
| | | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCCACTTGGTATGTGGTCTAGGCTGATAATATTCGGCGTTATGGCCGGCG |
| | | TGATCGGTACAATTCTGCTCATCAGTTATGGGATACGGAGATTGTGACTGTGCCTTCTAGTTGCCAGCCAT |
| | | CTGTTGTTTGCCCCTCCCCCGTGCCTTCCTTGACCCTGGAAGGTGCCACTCCCACTGTCCTTTCCTAATAA |
| | | AATGAGGAAATTGCATCGCATTGTCTGAGTAGGTGTCATTCTATTCTGGGGGGTGGGGTGGGGCAGGACAG |
| | | CAAGGGGGAGGATTGGGAAGACAATAGCAGGCATGCTGGGGAtggccatgcttcttgccccttgggcctcc |
| | | ccccagcccctcctccccttcctgcacccgtacccccgtggtctttgaataaagtctgagtgggggcagcc |
| | | tgtgtgtgcctgagttttttccctcagcaaacgtgccaggcatgggcgtggacagcagctgggacacacat |
| | | ggctagaacctctctgcagctggatagggtaggaaaaggcaggggcgggaggaggggatggaggagggaaa |
| | | gtggagccaccgcgaagtccagctggaaaaacgctggaccctagagtgctttgaggatgcatttgctcttt |
| | | cccgagttttattcccagacttttcagattcaatgcaggtttgctgaaataatgaatttatccatctttac |
| | | gtttctgggcactctgtgccaagaactggctggctttctgcctgggacgtcactggtttcccagaggtcct |
| | | cccacatatgggtggtgggtaggtcagagaagtcccactccagcatggctgcattgatcccccategttcc |
| | | cactagtctccgtaaaacctcccagatacaggcacagtctagatgaaatcaggggtgcggggtgcaactgc |
| | | aggccccaggcaattcaataggggctctactttcacccccaggtcaccccagaatgctcacacaccagaca |
| | | ctgacgccctggggctgtcaagatcaggcgtttgtctctgggcccagctcagggcccagctcagcacccac |
| | | tcagctcccctgaggctggggagcctgtcccattgcgactggagaggagagcggggccacagaggcctggc |
| | | tagaaggtcccttctccctggtgtgtgttttctctctgctgagcaggcttgcagtgcctggggtatca |
| 24 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | cleavable | gaggtgtagcgcaggcggcggctgcgggcctgggccctcggccccactgCCGCCCACCTCCCCGCCGAGTT |
| | linker-GPA- | CACCCCTGCGGTGCACGCCaccctcttctctgcacagCTCCTAAGCCACTGCCTGCTGGTGACCCTGGTCC |
| | GPA(C-term)- | CTGGACAAGTTCCTGGCTTCTGTGAGCACCGTGCTGACCTCCAAATACCGTGAGGGCAGGGGAAGTCTTCT |
| | 3′ homology | AACATGCGGGGACGTGGAGGAAAATCCCGGCCCCATGTACGGGAAGATTATTTTCGTGTTGTTGCTCAGTG |
| | arm | AGATCGTTTCTATCTCCGCTGCCATCACGACCAAGACCATCCCACGTTTAATAAAATAACAGAAGACCCTC |
| | | AAGGCGACGCCGCTCAAAAGACGGACACGACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCCACTTGGTATGTGGTCTAGGCTGATAATATTCGGCGTTATGGCCGGCG |
| | | TGATCGGTACAATTCTGCTCATCAGTTATGGGATACGGAGATTGATCAAGAAGAGTCCTTCCGACGTCAAG |
| | | CCACTGCCAAGCCCAGATACCGATGTTCCGCTTTCTTCAGTGGAGATTGAGAACCCCGAAACTTCTGACCA |
| | | GTGAtggccatgcttcttgccccttgggcctccccccagcccctectccccttcctgcacccgtacccccg |
| | | tggtctttgaataaagtctgagtgggcggcagcctgtgtgtgcctgagttttttccctcagcaaacgtgcc |
| | | aggcatgggcgtggacagcagctgggacacacatggctagaacctctctgcagctggatagggtaggaaaa |
| | | ggcaggggcgggaggaggggatggaggagggaaagtggagccaccgcgaagtccagctggaaaaacgctgg |
| | | accctagagtgctttgaggatgcatttgctctttcccgagttttattcccagacttttcagattcaatgca |
| | | ggtttgctgaaataatgaatttatccatctttacgtttctgggcactctgtgccaagaactggctggcttt |
| | | ctgcctgggacgtcactggtttcccagaggtcctcccacatatgggtggtgggtaggtcagagaagtccca |
| | | ctccagcatggctgcattgatcccccatcgttcccactagtctccgtaaaacctcccagatacaggcacag |
| | | tctagatgaaatcaggggtgcggggtgcaactgcaggccccaggcaattcaataggggctctactttcacc |
| | | cccaggtcaccccagaatgctcacacaccagacactgacgccctggggctgtcaagatcaggcgtttgtct |
| | | ctgggcccagctcagggcccagctcagcacccactcagctcccctgaggctggggagcctgtcccattgcg |
| | | actggagaggagagcggggccacagaggcctggctagaaggtcccttctccctggtgtgtgttttctctct |
| | | gctgagcaggcttgcagtgcctggggtatca |
| 25 | 5′ homology | GTTCCTGTCCTTCCCCACCACCAAGACCTACTTCCCGCACTTCGACCTGAGCCACGGCTCTGCCCAGGTTA |
| | arm-2A- | AGGGCCACGGCAAGAAGGTGGCCGACGCGCTGACCAACGCCGTGGCGCACGTGGACGACATGCCCAACGCG |
| | therapeutic | CTGTCCGCCCTGAGCGACCTGCACGCGCACAAGCTTCGGGTGGACCCGGTCAACTTCAAGgtgagcggcgg |
| | protein- | gccgggagcgatctgggtcgaggggcgagatggcgccttcctcgcagggcagaggatcacgcgggttgcgg |
| | cleavable | gaggtgtagcgcaggcggcggctgcgggcctgggccctcggccccactgaccctcttctctgcacagCTCC |
| | linker-GPA- | TAAGCCACTGCCTGCTGGTGACCCTGGCCGCCCACCTCCCCGCCGAGTTCACCCCTGCGGTGCACGCCTCC |
| | GPA(C-term)- | CTGGACAAGTTCCTGGCTTCTGTGAGCACCGTGCTGACCTCCAAATACCGTGAGGGCAGGGGAAGTCTTCT |
| | pA- | AACATGCGGGGACGTGGAGGAAAATCCCGGCCCCATGTACGGGAAGATTATTTTCGTGTTGTTGCTCAGTG |
| | 3′ homology | AGATCGTTTCTATCTCCGCTGAAGACCCTCAAGGCGACGCCGCTCAAAAGACGGACACGAGCCATCACGAC |
| | arm | CAAGACCATCCCACGTTTAATAAAATAACACCTAATCTCGCCGAATTTGCATTTTCACTGTATAGGCAACT |
| | | CGCCCATCAAAGCAATTCTACAAACATTTTCTTTAGCCCGGTCAGTATAGCGACTGCTTTCGCCATGCTGT |
| | | CTCTCGGTACAAAAGCCGATACCCATGACGAGATTTTGGAAGGACTCAACTTTAATCTGACCGAAATACCC |
| | | GAAGCACAAATTCACGAGGGGTTTCAAGAGCTGCTGCGAACTTTGAATCAACCCGATTCCCAACTGCAACT |
| | | CACGACAGGTAACGGGTTGTTTCTGAGTGAAGGGCTTAAACTGGTTGACAAATTCCTGGAAGACGTGAAGA |
| | | AACTCTATCATAGTGAGGCATTTACAGTTAATTTTGGAGATACCGAGGAAGCTAAGAAGCAAATTAATGAC |
| | | TATGTTGAGAAAGGCACGCAGGGAAAGATCGTTGACCTCGTGAAAGAACTGGATCGAGATACGGTGTTCGC |
| | | CCTCGTCAACTATATATTCTTCAAGGGGAAGTGGGAACGCCCATTCGAGGTGAAAGATACAGAAGAGGAAG |
| | | ATTTTCATGTAGATCAAGTTACAACAGTAAAAGTACCCATGATGAAAAGACTCGGGATGTTCAATATCCAA |
| | | CATTGCAAGAAGTTGTCATCTTGGGTCCTTTTGATGAAGTATCTTGGGAACGCAACGGCTATATTCTTTCT |
| | | CCCGGACGAAGGCAAGCTGCAACATCTCGAGAACGAGCTGACTCATGACATTATTACGAAATTTCTTGAGA |
| | | ACGAGGATCGCCGGAGCGCGTCCCTGCACCTGCCTAAGCTCAGCATAACAGGTACGTACGACCTCAAATCC |
| | | GTGTTGGGACAGTTGGGGATTACGAAAGTGTTTTCCAACGGAGCGGATCTGAGCGGTGTGACCGAAGAAGC |
| | | TCCACTTAAACTGTCAAAAGCGGTCCACAAAGCCGTTTTGACTATAGATGAAAAGGGTACAGAGGCCGCCG |
| | | GCGCGATGTTCCTGGAAGCTATCCCGATGTCCATACCACCAGAAGTGAAATTTAATAAGCCTTTCGTGTTT |
| | | CTGATGATAGAGCAGAACACAAAATCCCCACTGTTTATGGGCAAGGTCGTCAACCCAACACAGAAGGGTTC |
| | | AGGCGGGTCCGGCGGAAGTGGGCCACTTGGTATGTGGTCTAGGCTGATAATATTCGGCGTTATGGCCGGCG |
| | | TGATCGGTACAATTCTGCTCATCAGTTATGGGATACGGAGATTGATCAAGAAGAGTCCTTCCGACGTCAAG |
| | | CCACTGCCAAGCCCAGATACCGATGTTCCGCTTTCTTCAGTGGAGATTGAGAACCCCGAAACTTCTGACCA |
| | | GTGACTGTGCCTTCTAGTTGCCAGCCATCTGTTGTTTGCCCCTCCCCCGTGCCTTCCTTGACCCTGGAAGG |
| | | TGCCACTCCCACTGTCCTTTCCTAATAAAATGAGGAAATTGCATCGCATTGTCTGAGTAGGTGTCATTCTA |
| | | TTCTGGGGGGTGGGGTGGGGCAGGACAGCAAGGGGGAGGATTGGGAAGACAATAGCAGGCATGCTGGGGAt |
| | | ggccatgcttcttgccccttgggcctccccccagcccctectccccttcctgcacccgtacccccgtggtc |
| | | tttgaataaagtctgagtgggcggcagcctgtgtgtgcctgagttttttccctcagcaaacgtgccaggca |
| | | tgggcgtggacagcagctgggacacacatggctagaacctctctgcagctggatagggtaggaaaaggcag |
| | | gggcgggaggaggggatggaggagggaaagtggagccaccgcgaagtccagctggaaaaacgctggaccct |
| | | agagtgctttgaggatgcatttgctctttcccgagttttattcccagacttttcagattcaatgcaggttt |
| | | gctgaaataatgaatttatccatctttacgtttctgggcactctgtgccaagaactggctggctttctgcc |
| | | tgggacgtcactggtttcccagaggtcctcccacatatgggtggtgggtaggtcagagaagtcccactcca |
| | | gcatggctgcattgatcccccatcgttcccactagtctccgtaaaacctcccagatacaggcacagtctag |
| | | atgaaatcaggggtgcggggtgcaactgcaggccccaggcaattcaataggggctctactttcacccccag |
| | | gtcaccccagaatgctcacacaccagacactgacgccctggggctgtcaagatcaggcgtttgtctctggg |
| | | cccagctcagggcccagctcagcacccactcagctcccctgaggctggggagcctgtcccattgcgactgg |
| | | agaggagagcggggccacagaggcctggctagaaggtcccttctccctggtgtgtgttttctctctgctga |
| | | gcaggcttgcagtgcctggggtatca |
| 26 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | 3′ homology | TAAGCCACTGCCTGCTGGTGACCCTGGCCGCCCACCTCCCCGCCGAGTTCACCCCTGCGGTGCACGCCTCC |
| | arm | CTGGACAAGTTCCTGGCTTCTGTGAGCACCGTGCTGACCTCCAAATACCGTCGGGCTAAGAGAGGCAGCGG |
| | | CGAGGGCAGGGGAAGTCTTCTAACATGCGGGGACGTGGAGGAAAATCCCGGCCCCATGCCTTCATCAGTAT |
| | | CTTGGGGAATACTGCTCCTTGCTGGGTTGTGTTGTCTCGTACCCGTGAGTCTCGCCGAAGACCCTCAAGGC |
| | | GACGCCGCACAAAAGACTGACACTTCTCATCACGACCAAGACCATCCTACATTTAATAAAATTACTCCAAA |
| | | TCTCGCCGAATTTGCGTTTTCTCTGTATAGGCAACTCGCTCACCAATCTAATTCAACGAACATATTCTTTT |
| | | CACCTGTTTCCATAGCCACCGCTTTCGCCATGCTGAGTTTGGGAACAAAAGCAGATACCCATGACGAGATA |
| | | CTCGAAGGACTCAACTTTAATCTGACAGAAATCCCTGAAGCACAAATTCACGAGGGTTTTCAAGAGCTGCT |
| | | GAGAACTTTGAATCAACCCGATTCCCAATTGCAACTCACAACAGGAAACGGTTTGTTTCTTTCAGAAGGGC |
| | | TCAAACTGGTCGACAAATTCCTCGAAGACGTGAAGAAACTTTATCATAGCGAGGCTTTTACCGTGAATTTT |
| | | GGAGATACGGAAGAAGCTAAGAAGCAAATAAATGACTATGTCGAAAAGGGGACACAGGGAAAGATAGTTGA |
| | | CCTGGTGAAAGAACTGGATAGGGATACTGTGTTCGCGCTCGTCAACTATATCTTCTTCAAGGGGAAGTGGG |
| | | AACGGCCATTCGAGGTTAAAGATACAGAAGAGGAAGATTTTCATGTAGATCAAGTCACAACAGTCAAAGTT |
| | | CCAATGATGAAACGCCTCGGGATGTTCAATATACAACATTGCAAGAAACTTAGCTCATGGGTCCTTTTGAT |
| | | GAAGTATCTCGGGAACGCTACAGCGATATTCTTTCTCCCAGACGAAGGTAAGCTGCAACATCTTGAGAACG |
| | | AGCTGACACATGACATAATAACAAAATTTCTTGAGAACGAGGATCGCCGGTCCGCATCCCTGCACCTGCCG |
| | | AAGCTTAGCATAACCGGCACATACGACTTGAAATCTGTTCTTGGGCAGCTTGGTATTACAAAAGTGTTTTC |
| | | CAACGGCGCGGATCTGTCAGGCGTGACGGAAGAAGCTCCTCTTAAACTGAGTAAAGCAGTCCACAAAGCAG |
| | | TACTCACTATTGATGAAAAGGGTACCGAGGCGGCCGGAGCTATGTTCCTCGAAGCTATTCCTATGAGTATT |
| | | CCCCCTGAAGTTAAATTTAATAAGCCTTTCGTGTTTCTCATGATAGAGCAGAACACGAAAAGCCCTCTGTT |
| | | TATGGGCAAGGTCGTCAACCCAACACAGAAGTAGtggccatgcttcttgccccttgggcctccccccagcc |
| | | cctcctccccttcctgcacccgtacccccgtggtctttgaataaagtctgagtgggcggcagcctgtgtgt |
| | | gcctgagttttttccctcagcaaacgtgccaggcatgggcgtggacagcagctgggacacacatggctaga |
| | | acctctctgcagctggatagggtaggaaaaggcaggggcgggaggaggggatggaggagggaaagtggagc |
| | | caccgcgaagtccagctggaaaaacgctggaccctagagtgctttgaggatgcatttgctctttcccgagt |
| | | tttattcccagacttttcagattcaatgcaggtttgctgaaataatgaatttatccatctttacgtttctg |
| | | ggcactctgtgccaagaactggctggctttctgcctgggacgtcactggtttcccagaggtcctcccacat |
| | | atggggggggtaggtcagagaagtcccactccagcatggctgcattgatcccccatcgttcccactagtct |
| | | ccgtaaaacctcccagatacaggcacagtctagatgaaatcaggggtgcggggtgcaactgcaggccccag |
| | | gcaattcaataggggctctactttcacccccaggtcaccccagaatgctcacacaccagacactgacgccc |
| | | tggggctgtcaagatcaggcgtttgtctctgggcccagctcagggcccagctcagcacccactcagctccc |
| | | ctgaggctggggagcctgtcccattgcgactggagaggagagcggggccacagaggcctggctagaaggtc |
| | | ccttctccctggtgtgtgttttctctctgctgagcaggcttgcagtgcctggggtatca |
| 27 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein-pA- | gaggtgtagcgcaggcggcggctgcgggcctgggccctcggccccactgaccctcttctctgcacagCTCC |
| | 3′ homology | TAAGCCACTGCCTGCTGGTGACCCTGGCCGCCCACCTCCCCGCCGAGTTCACCCCTGCGGTGCACGCCTCC |
| | arm | CTGGACAAGTTCCTGGCTTCTGTGAGCACCGTGCTGACCTCCAAATACCGTCGGGCTAAGAGAGGCAGCGG |
| | | CGAGGGCAGGGGAAGTCTTCTAACATGCGGGGACGTGGAGGAAAATCCCGGCCCCATGCCTTCATCAGTAT |
| | | CTTGGGGAATACTGCTCCTTGCTGGGTTGTGTTGTCTCGTACCCGTGAGTCTCGCCGAAGACCCTCAAGGC |
| | | GACGCCGCACAAAAGACTGACACTTCTCATCACGACCAAGACCATCCTACATTTAATAAAATTACTCCAAA |
| | | TCTCGCCGAATTTGCGTTTTCTCTGTATAGGCAACTCGCTCACCAATCTAATTCAACGAACATATTCTTTT |
| | | CACCTGTTTCCATAGCCACCGCTTTCGCCATGCTGAGTTTGGGAACAAAAGCAGATACCCATGACGAGATA |
| | | CTCGAAGGACTCAACTTTAATCTGACAGAAATCCCTGAAGCACAAATTCACGAGGGTTTTCAAGAGCTGCT |
| | | GAGAACTTTGAATCAACCCGATTCCCAATTGCAACTCACAACAGGAAACGGTTTGTTTCTTTCAGAAGGGC |
| | | TCAAACTGGTCGACAAATTCCTCGAAGACGTGAAGAAACTTTATCATAGCGAGGCTTTTACCGTGAATTTT |
| | | GGAGATACGGAAGAAGCTAAGAAGCAAATAAATGACTATGTCGAAAAGGGGACACAGGGAAAGATAGTTGA |
| | | CCTGGTGAAAGAACTGGATAGGGATACTGTGTTCGCGCTCGTCAACTATATCTTCTTCAAGGGGAAGTGGG |
| | | AACGGCCATTCGAGGTTAAAGATACAGAAGAGGAAGATTTTCATGTAGATCAAGTCACAACAGTCAAAGTT |
| | | CCAATGATGAAACGCCTCGGGATGTTCAATATACAACATTGCAAGAAACTTAGCTCATGGGTCCTTTTGAT |
| | | GAAGTATCTCGGGAACGCTACAGCGATATTCTTTCTCCCAGACGAAGGTAAGCTGCAACATCTTGAGAACG |
| | | AGCTGACACATGACATAATAACAAAATTTCTTGAGAACGAGGATCGCCGGTCCGCATCCCTGCACCTGCCG |
| | | AAGCTTAGCATAACCGGCACATACGACTTGAAATCTGTTCTTGGGCAGCTTGGTATTACAAAAGTGTTTTC |
| | | CAACGGCGCGGATCTGTCAGGCGTGACGGAAGAAGCTCCTCTTAAACTGAGTAAAGCAGTCCACAAAGCAG |
| | | TACTCACTATTGATGAAAAGGGTACCGAGGCGGCCGGAGCTATGTTCCTCGAAGCTATTCCTATGAGTATT |
| | | CCCCCTGAAGTTAAATTTAATAAGCCTTTCGTGTTTCTCATGATAGAGCAGAACACGAAAAGCCCTCTGTT |
| | | TATGGGCAAGGTCGTCAACCCAACACAGAAGTAGCTGTGCCTTCTAGTTGCCAGCCATCTGTTGTTTGCCC |
| | | CTCCCCCGTGCCTTCCTTGACCCTGGAAGGTGCCACTCCCACTGTCCTTTCCTAATAAAATGAGGAAATTG |
| | | CATCGCATTGTCTGAGTAGGTGTCATTCTATTCTGGGGGGTGGGGTGGGGCAGGACAGCAAGGGGGAGGAT |
| | | TGGGAAGACAATAGCAGGCATGCTGGGGAtggccatgcttcttgccccttgggcctccccccagcccctcc |
| | | tccccttcctgcacccgtacccccgtggtctttgaataaagtctgagtgggcggcagcctgtgtgtgcctg |
| | | agttttttccctcagcaaacgtgccaggcatgggcgtggacagcagctgggacacacatggctagaacctc |
| | | tctgcagctggatagggtaggaaaaggcaggggcgggaggaggggatggaggagggaaagtggagccaccg |
| | | cgaagtccagctggaaaaacgctggaccctagagtgctttgaggatgcatttgctctttcccgagttttat |
| | | tcccagacttttcagattcaatgcaggtttgctgaaataatgaatttatccatctttacgtttctgggcac |
| | | tctgtgccaagaactggctggctttctgcctgggacgtcactggtttcccagaggtcctcccacatatggg |
| | | tggtgggtaggtcagagaagtcccactccagcatggctgcattgatcccccatcgttcccactagtctccg |
| | | taaaacctcccagatacaggcacagtctagatgaaatcaggggtgcggggtgcaactgcaggccccaggca |
| | | attcaataggggctctactttcacccccaggtcaccccagaatgctcacacaccagacactgacgccctgg |
| | | ggctgtcaagatcaggcgtttgtctctgggcccagctcagggcccagctcagcacccactcagctcccctg |
| | | aggctggggagcctgtcccattgcgactggagaggagagcggggccacagaggcctggctagaaggtccct |
| | | tctccctggtgtgtgttttctctctgctgagcaggcttgcagtgcctggggtatca |
| 28 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | noncleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | 3′ homology | CGAGGGCAGGGGAAGTCTTCTAACATGCGGGGACGTGGAGGAAAATCCCGGCCCCATGTACGGGAAGATTA |
| | arm | TTTTCGTGTTGTTGCTCAGTGAGATCGTTTCTATCTCCGCTGAAGACCCTCAAGGCGACGCCGCTCAAAAG |
| | | ACGGACACGAGCCATCACGACCAAGACCATCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCTGATAATATTCGGCGTTATGGCCGGCG |
| | | TGATCGGTACAATTCTGCTCATCAGTTATGGGATACGGAGATTGTGAtggccatgcttcttgccccttggg |
| | | cctccccccagcccctcctccccttcctgcacccgtacccccgtggtctttgaataaagtctgagtgggcg |
| | | gcagcctgtgtgtgcctgagttttttccctcagcaaacgtgccaggcatgggcgtggacagcagctgggac |
| | | acacatggctagaacctctctgcagctggatagggtaggaaaaggcaggggcgggaggaggggatggagga |
| | | gggaaagtggagccaccgcgaagtccagctggaaaaacgctggaccctagagtgctttgaggatgcatttg |
| | | ctctttcccgagttttattcccagacttttcagattcaatgcaggtttgctgaaataatgaatttatccat |
| | | ctttacgtttctgggcactctgtgccaagaactggctggctttctgcctgggacgtcactggtttcccaga |
| | | ggtcctcccacatatgggtggtgggtaggtcagagaagtcccactccagcatggctgcattgatcccccat |
| | | cgttcccactagtctccgtaaaacctcccagatacaggcacagtctagatgaaatcaggggtgcggggtgc |
| | | aactgcaggccccaggcaattcaataggggctctactttcacccccaggtcaccccagaatgctcacacac |
| | | cagacactgacgccctggggctgtcaagatcaggcgtttgtctctgggcccagctcagggcccagctcagc |
| | | acccactcagctcccctgaggctggggagcctgtcccattgcgactggagaggagagcggggccacagagg |
| | | cctggctagaaggtcccttctccctggtgtgtgttttctctctgctgagcaggcttgcagtgcctggggta |
| | | tca |
| 29 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | noncleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | pA- | CGAGGGCAGGGGAAGTCTTCTAACATGCGGGGACGTGGAGGAAAATCCCGGCCCCATGTACGGGAAGATTA |
| | 3′ homology | TTTTCGTGTTGTTGCTCAGTGAGATCGTTTCTATCTCCGCTGAAGACCCTCAAGGCGACGCCGCTCAAAAG |
| | arm | ACGGACACGAGCCATCACGACCAAGACCATCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCTGATAATATTCGGCGTTATGGCCGGCG |
| | | TGATCGGTACAATTCTGCTCATCAGTTATGGGATACGGAGATTGTGACTGTGCCTTCTAGTTGCCAGCCAT |
| | | CTGTTGTTTGCCCCTCCCCCGTGCCTTCCTTGACCCTGGAAGGTGCCACTCCCACTGTCCTTTCCTAATAA |
| | | AATGAGGAAATTGCATCGCATTGTCTGAGTAGGTGTCATTCTATTCTGGGGGGTGGGGTGGGGCAGGACAG |
| | | CAAGGGGGAGGATTGGGAAGACAATAGCAGGCATGCTGGGGAtggccatgcttcttgccccttgggcctcc |
| | | ccccagcccctectccccttcctgcaccegtacccccgtggtctttgaataaagtctgagtgggcggcagc |
| | | ctgtgtgtgcctgagttttttccctcagcaaacgtgccaggcatgggcgtggacagcagctgggacacaca |
| | | tggctagaacctctctgcagctggatagggtaggaaaaggcaggggcgggaggaggggatggaggagggaa |
| | | agtggagccaccgcgaagtccagctggaaaaacgctggaccctagagtgctttgaggatgcatttgctctt |
| | | tcccgagttttattcccagacttttcagattcaatgcaggtttgctgaaataatgaatttatccatcttta |
| | | cgtttctgggcactctgtgccaagaactggctggctttctgcctgggacgtcactggtttcccagaggtcc |
| | | tcccacatatgggtggtgggtaggtcagagaagtcccactccagcatggctgcattgatcccccatcgttc |
| | | ccactagtctccgtaaaacctcccagatacaggcacagtctagatgaaatcaggggtgcggggtgcaactg |
| | | caggccccaggcaattcaataggggctctactttcacccccaggtcaccccagaatgctcacacaccagac |
| | | actgacgccctggggctgtcaagatcaggcgtttgtctctgggcccagctcagggcccagctcagcaccca |
| | | ctcagctcccctgaggctggggagcctgtcccattgcgactggagaggagagcggggccacagaggcctgg |
| | | ctagaaggtcccttctccctggtgtgtgttttctctctgctgagcaggcttgcagtgcctggggtatca |
| 30 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- - | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | noncleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | GPA(C-term)- | CGAGGGCAGGGGAAGTCTTCTAACATGCGGGGACGTGGAGGAAAATCCCGGCCCCATGTACGGGAAGATTA |
| | 3′ homology | TTTTCGTGTTGTTGCTCAGTGAGATCGTTTCTATCTCCGCTGAAGACCCTCAAGGCGACGCCGCTCAAAAG |
| | arm | ACGGACACGAGCCATCACGACCAAGACCATCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCTGATAATATTCGGCGTTATGGCCGGCG |
| | | TGATCGGTACAATTCTGCTCATCAGTTATGGGATACGGAGATTGATCAAGAAGAGTCCTTCCGACGTCAAG |
| | | CCACTGCCAAGCCCAGATACCGATGTTCCGCTTTCTTCAGTGGAGATTGAGAACCCCGAAACTTCTGACCA |
| | | GTGAtggccatgcttcttgccccttgggcctccccccagcccctcctccccttcctgcacccgtacccccg |
| | | tggtctttgaataaagtctgagtgggcggcagcctgtgtgtgcctgagttttttccctcagcaaacgtgcc |
| | | aggcatgggcgtggacagcagctgggacacacatggctagaacctctctgcagctggatagggtaggaaaa |
| | | ggcaggggcgggaggaggggatggaggagggaaagtggagccaccgcgaagtccagctggaaaaacgctgg |
| | | accctagagtgctttgaggatgcatttgctctttcccgagttttattcccagacttttcagattcaatgca |
| | | ggtttgctgaaataatgaatttatccatctttacgtttctgggcactctgtgccaagaactggctggcttt |
| | | ctgcctgggacgtcactggtttcccagaggtcctcccacatatggggggggtaggtcagagaagtcccact |
| | | ccagcatggctgcattgatcccccatcgttcccactagtctccgtaaaacctcccagatacaggcacagtc |
| | | tagatgaaatcaggggtgcggggtgcaactgcaggccccaggcaattcaataggggctctactttcacccc |
| | | caggtcaccccagaatgctcacacaccagacactgacgccctggggctgtcaagatcaggcgtttgtctct |
| | | gggcccagctcagggcccagctcagcacccactcagctcccctgaggctggggagcctgtcccattgcgac |
| | | tggagaggagagcggggccacagaggcctggctagaaggtcccttctccctggtgtgtgttttctctctgc |
| | | tgagcaggcttgcagtgcctggggtatca |
| 31 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- - | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | noncleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | GPA(C-term)- | CGAGGGCAGGGGAAGTCTTCTAACATGCGGGGACGTGGAGGAAAATCCCGGCCCCATGTACGGGAAGATTA |
| | pA- | TTTTCGTGTTGTTGCTCAGTGAGATCGTTTCTATCTCCGCTGAAGACCCTCAAGGCGACGCCGCTCAAAAG |
| | 3′ homology | ACGGACACGAGCCATCACGACCAAGACCATCCCACGTTTAATAAAATAACACCTAATCTCGCCGAATTTGC |
| | arm | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCTGATAATATTCGGCGTTATGGCCGGCG |
| | | TGATCGGTACAATTCTGCTCATCAGTTATGGGATACGGAGATTGATCAAGAAGAGTCCTTCCGACGTCAAG |
| | | CCACTGCCAAGCCCAGATACCGATGTTCCGCTTTCTTCAGTGGAGATTGAGAACCCCGAAACTTCTGACCA |
| | | GTGACTGTGCCTTCTAGTTGCCAGCCATCTGTTGTTTGCCCCTCCCCCGTGCCTTCCTTGACCCTGGAAGG |
| | | TGCCACTCCCACTGTCCTTTCCTAATAAAATGAGGAAATTGCATCGCATTGTCTGAGTAGGTGTCATTCTA |
| | | TTCTGGGGGGTGGGGTGGGGCAGGACAGCAAGGGGGAGGATTGGGAAGACAATAGCAGGCATGCTGGGGAt |
| | | ggccatgcttcttgccccttgggcctccccccagcccctectccccttcctgcacccgtacccccgtggtc |
| | | tttgaataaagtctgagtgggcggcagcctgtgtgtgcctgagttttttccctcagcaaacgtgccaggca |
| | | tgggcgtggacagcagctgggacacacatggctagaacctctctgcagctggatagggtaggaaaaggcag |
| | | gggcgggaggaggggatggaggagggaaagtggagccaccgcgaagtccagctggaaaaacgctggaccct |
| | | agagtgctttgaggatgcatttgctctttcccgagttttattcccagacttttcagattcaatgcaggttt |
| | | gctgaaataatgaatttatccatctttacgtttctgggcactctgtgccaagaactggctggctttctgcc |
| | | tgggacgtcactggtttcccagaggtcctcccacatatggggggggtaggtcagagaagtcccactccagc |
| | | atggctgcattgatcccccatcgttcccactagtctccgtaaaacctcccagatacaggcacagtctagat |
| | | gaaatcaggggtgcggggtgcaactgcaggccccaggcaattcaataggggctctactttcacccccaggt |
| | | caccccagaatgctcacacaccagacactgacgccctggggctgtcaagatcaggcgtttgtctctgggcc |
| | | cagctcagggcccagctcagcacccactcagctcccctgaggctggggagcctgtcccattgcgactggag |
| | | aggagagcggggccacagaggcctggctagaaggtcccttctccctggtgtgtgttttctctctgctgagc |
| | | aggcttgcagtgcctggggtatca |
| 32 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | cleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | 3′ homology | CGAGGGCAGGGGAAGTCTTCTAACATGCGGGGACGTGGAGGAAAATCCCGGCCCCATGTACGGGAAGATTA |
| | arm | TTTTCGTGTTGTTGCTCAGCCGCTCAAAAGACGGACACGAGCCATCACGACCAAGACCATGAGATCGTTTC |
| | | TATCTCCGCTGAAGACCCTCAAGGCGACGTCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCCACTTGGTATGTGGTCTAGGCTGATAA |
| | | TATTCGGCGTTATGGCCGGCGTGATCGGTACAATTCTGCTCATCAGTTATGGGATACGGAGATTGTGAtgg |
| | | ccatgcttcttgccccttgggcctccccccagcccctcctccccttcctgcacccgtacccccgtggtctt |
| | | tgaataaagtctgagtgggcggcagcctgtgtgtgcctgagttttttccctcagcaaacgtgccaggcatg |
| | | ggcgtggacagcagctgggacacacatggctagaacctctctgcagctggatagggtaggaaaaggcaggg |
| | | gcgggaggaggggatggaggagggaaagtggagccaccgcgaagtccagctggaaaaacgctggaccctag |
| | | agtgctttgaggatgcatttgctctttcccgagttttattcccagacttttcagattcaatgcaggtttgc |
| | | tgaaataatgaatttatccatctttacgtttctgggcactctgtgccaagaactggctggctttctgcctg |
| | | ggacgtcactggtttcccagaggtcctcccacatatggggggggtaggtcagagaagtcccactccagcat |
| | | ggctgcattgatcccccatcgttcccactagtctccgtaaaacctcccagatacaggcacagtctagatga |
| | | aatcaggggtgcggggtgcaactgcaggccccaggcaattcaataggggctctactttcacccccaggtca |
| | | ccccagaatgctcacacaccagacactgacgccctggggctgtcaagatcaggcgtttgtctctgggccca |
| | | gctcagggcccagctcagcacccactcagctcccctgaggctggggagcctgtcccattgcgactggagag |
| | | gagagcggggccacagaggcctggctagaaggtcccttctccctggtgtgtgttttctctctgctgagcag |
| | | gcttgcagtgcctggggtatca |
| 33 | 5′ homology | GTTCCTGTCCTTCCCCACCACCAAGACCTACTTCCCGCACTTAGAAGGTGGCCGACGCGCTGACCAACGCC |
| | arm-Furin- | GTGGCGCACGTCGACCTGAGCCACGGCTCTGCCCAGGTTAAGGGCCACGGCAGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | cleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | pA- | CGAGGGCAGGGGAAGTCTTCTAACATGCGGGGACGTGGAGGAAAATCCCGGCCCCATGTACGGGAAGATTA |
| | 3′ homology | TTTTCGTGTTGTTGCTCAGTGAGATCGTTTCTATCTCCGCTGAAGACCCTCAAGGCGACGCCGCTCAAAAG |
| | arm | ACGGACACGAGCCATCACGACCAAGACCATCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCCACTTGGTATGTGGTCTAGGCTGATAA |
| | | TATTCGGCGTTATGGCCGGCGTGATCGGTACAATTCTGCTCATCAGTTATGGGATACGGAGATTGTGACTG |
| | | TGCCTTCTAGTTGCCAGCCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGGTGTCATTCTATTCTGGG |
| | | GGGTGGGGTGGGGCAGGACAGCAAGGGGGAGGATTGGGAAGACAATAGCAGGCATGCTGGGGAtggccatg |
| | | cttcttgccccttgggcctccccccagcccctcctccccttcctgcacccgtacccccgtggtctttgaat |
| | | aaagtctgagtgggcggcagcctgtgtgtgcctgagttttttccctcagcaaacgtgccaggcatgggcgt |
| | | ggacagcagctgggacacacatggctagaacctctctgcagctggatagggtaggaaaaggcaggggcggg |
| | | aggaggggatggaggagggaaagtggagccaccgcgaagtccagctggaaaaacgctggaccctagagtgc |
| | | tttgaggatgcatttgctctttcccgagttttattcccagacttttcagattcaatgcaggtttgctgaaa |
| | | taatgaatttatccatctttacgtttctgggcactctgtgccaagaactggctggctttctgcctgggacg |
| | | tcactggtttcccagaggtcctcccacatatgggtggtgggtaggtcagagaagtcccactccagcatggc |
| | | tgcattgatcccccatcgttcccactagtctccgtaaaacctcccagatacaggcacagtctagatgaaat |
| | | caggggtgcggggtgcaactgcaggccccaggcaattcaataggggctctactttcacccccaggtcaccc |
| | | cagaatgctcacacaccagacactgacgccctggggctgtcaagatcaggcgtttgtctctgggcccagct |
| | | cagggcccagctcagcacccactcagctcccctgaggctggggagcctgtcccattgcgactggagaggag |
| | | agcggggccacagaggcctggctagaaggtcccttctccctggtgtgtgttttctctctgctgagcaggct |
| | | tgcagtgcctggggtatca |
| 34 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | cleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | GPA(C-term)- | CGAGGGCAGGGGAAGTCTTCTAACATGCGGGGACGTGGAGGAAAATCCCGGCCCCATGTACGGGAAGATTA |
| | 3′ homology | TTTTCGTGTTGTTGCTCAGTGAGATCGTTTCTATCTCCGCTGAAGACCCTCAAGGCGACGCCGCTCAAAAG |
| | arm | ACGGACACGAGCCATCACGACCAAGACCATCCCACGTTTAATAAAATAACACCTAATCTCGCCGAATTTGC |
| | | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCCACTTGGTATGTGGTCTAGGCTGATAA |
| | | TATTCGGCGTTATGGCCGGCGTGATCGGTACAATTCTGCTCATCAGTTATGGGATACGGAGATTGATCAAG |
| | | AAGAGTCCTTCCGACGTCAAGCCACTGCCAAGCCCAGATACCGATGTTCCGCTTTCTTCAGTGGAGATTGA |
| | | GAACCCCGAAACTTCTGACCAGTGAtggccatgcttcttgccccttgggcctccccccagcccctcctccc |
| | | cttcctgcacccgtacccccgtggtctttgaataaagtctgagtgggcggcagcctgtgtgtgcctgagtt |
| | | ttttccctcagcaaacgtgccaggcatgggcgtggacagcagctgggacacacatggctagaacctctctg |
| | | cagctggatagggtaggaaaaggcaggggcgggaggaggggatggaggagggaaagtggagccaccgcgaa |
| | | gtccagctggaaaaacgctggaccctagagtgctttgaggatgcatttgctctttcccgagttttattccc |
| | | agacttttcagattcaatgcaggtttgctgaaataatgaatttatccatctttacgtttctgggcactctg |
| | | tgccaagaactggctggctttctgcctgggacgtcactggtttcccagaggtcctcccacatatgggtggt |
| | | gggtaggtcagagaagtcccactccagcatggctgcattgatcccccatcgttcccactagtctccgtaaa |
| | | acctcccagatacaggcacagtctagatgaaatcaggggtgcggggtgcaactgcaggccccaggcaattc |
| | | aataggggctctactttcacccccaggtcaccccagaatgctcacacaccagacactgacgccctggggct |
| | | gtcaagatcaggcgtttgtctctgggcccagctcagggcccagctcagcacccactcagctcccctgaggc |
| | | tggggagcctgtcccattgcgactggagaggagagcggggccacagaggcctggctagaaggtcccttctc |
| | | cctggtgtgtgttttctctctgctgagcaggcttgcagtgcctggggtatca |
| 35 | 5′ homology | GTTCCTGTCCTTCCCCACCACCAAGACCTACTTCCCGCACTTCGACCTGAGCCACGGCTCTGCCCAGGTTA |
| | arm-Furin- | AGGGCCACGGCAAGAAGGTGGCCGACGCGCTGACCAACGCCGTGGCGCACGTGGACGACATGCCCAACGCG |
| | 2A- | CTGTCCGCCCTGAGCGACCTGCACGCGCACAAGCTTCGGGTGGACCCGGTCAACTTCAAGgtgagcggcgg |
| | therapeutic | gccgggagcgatctgggtcgaggggcgagatggcgccttcctcgcagggcagaggatcacgcgggttgcgg |
| | protein- | gaggtgtagcgcaggcggcggctgcgggcctgggccctcggccccactgaccctcttctctgcacagCTCC |
| | cleavable | TAAGCCACTGCCTGCTGGTGACCCTGGCCGCCCACCTCCCCGCCGAGTTCACCCCTGCGGTGCACGCCTCC |
| | linker-GPA- | CTGGACAAGTTCCTGGCTTCTGTGAGCACCGTGCTGACCTCCAAATACCGTCGGGCTAAGAGAGGCAGCGG |
| | GPA(C-term)- | CGAGGGCAGGGGAAGTCTTCTAACATGCGGGGACGTGGAGGAAAATCCCGGCCCCATGTACGGGAAGATTA |
| | pA- | TTTTCGTGTTGTTGCTCAGTGAGATCGTTTCTATCTCCGCTGAAGACCCTCAAGGCGACGCCGCTCAAAAG |
| | 3′ homology | ACGGACACGAGCCATCACGACCAAGACCATCCCACGTTTAATAAAATAACACCTAATCTCGCCGAATTTGC |
| | arm | ATTTTCACTGTATAGGCAACTCGCCCATCAAAGCAATTCTACAAACATTTTCTTTAGCCCGGTCAGTATAG |
| | | CGACTGCTTTCGCCATGCTGTCTCTCGGTACAAAAGCCGATACCCATGACGAGATTTTGGAAGGACTCAAC |
| | | TTTAATCTGACCGAAATACCCGAAGCACAAATTCACGAGGGGTTTCAAGAGCTGCTGCGAACTTTGAATCA |
| | | ACCCGATTCCCAACTGCAACTCACGACAGGTAACGGGTTGTTTCTGAGTGAAGGGCTTAAACTGGTTGACA |
| | | AATTCCTGGAAGACGTGAAGAAACTCTATCATAGTGAGGCATTTACAGTTAATTTTGGAGATACCGAGGAA |
| | | GCTAAGAAGCAAATTAATGACTATGTTGAGAAAGGCACGCAGGGAAAGATCGTTGACCTCGTGAAAGAACT |
| | | GGATCGAGATACGGTGTTCGCCCTCGTCAACTATATATTCTTCAAGGGGAAGTGGGAACGCCCATTCGAGG |
| | | TGAAAGATACAGAAGAGGAAGATTTTCATGTAGATCAAGTTACAACAGTAAAAGTACCCATGATGAAAAGA |
| | | CTCGGGATGTTCAATATCCAACATTGCAAGAAGTTGTCATCTTGGGTCCTTTTGATGAAGTATCTTGGGAA |
| | | CGCAACGGCTATATTCTTTCTCCCGGACGAAGGCAAGCTGCAACATCTCGAGAACGAGCTGACTCATGACA |
| | | TTATTACGAAATTTCTTGAGAACGAGGATCGCCGGAGCGCGTCCCTGCACCTGCCTAAGCTCAGCATAACA |
| | | GGTACGTACGACCTCAAATCCGTGTTGGGACAGTTGGGGATTACGAAAGTGTTTTCCAACGGAGCGGATCT |
| | | GAGCGGTGTGACCGAAGAAGCTCCACTTAAACTGTCAAAAGCGGTCCACAAAGCCGTTTTGACTATAGATG |
| | | AAAAGGGTACAGAGGCCGCCGGCGCGATGTTCCTGGAAGCTATCCCGATGTCCATACCACCAGAAGTGAAA |
| | | TTTAATAAGCCTTTCGTGTTTCTGATGATAGAGCAGAACACAAAATCCCCACTGTTTATGGGCAAGGTCGT |
| | | CAACCCAACACAGAAGGGTTCAGGCGGGTCCGGCGGAAGTGGGCCACTTGGTATGTGGTCTAGGCTGATAA |
| | | TATTCGGCGTTATGGCCGGCGTGATCGGTACAATTCTGCTCATCAGTTATGGGATACGGAGATTGATCAAG |
| | | AAGAGTCCTTCCGACGTCAAGCCACTGCCAAGCCCAGATACCGATGTTCCGCTTTCTTCAGTGGAGATTGA |
| | | GAACCCCGAAACTTCTGACCAGTGACTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAATAAAATGAGGAAATTGCATCGCATT |
| | | GTCTGAGTAGGTGTCATTCTATTCTGGGGGGTGGGGTGGGGCAGGACAGCAAGGGGGAGGATTGGGAAGAC |
| | | AATAGCAGGCATGCTGGGGAtggccatgcttcttgccccttgggcctccccccagcccctcctccccttcc |
| | | tgcacccgtacccccgtggtctttgaataaagtctgagtgggggcagcctgtgtgtgcctgagttttttcc |
| | | ctcagcaaacgtgccaggcatgggcgtggacagcagctgggacacacatggctagaacctctctgcagctg |
| | | gatagggtaggaaaaggcaggggcgggaggaggggatggaggagggaaagtggagccaccgcgaagtccag |
| | | ctggaaaaacgctggaccctagagtgctttgaggatgcatttgctctttcccgagttttattcccagactt |
| | | ttcagattcaatgcaggtttgctgaaataatgaatttatccatctttacgtttctgggcactctgtgccaa |
| | | gaactggctggctttctgcctgggacgtcactggtttcccagaggtcctcccacatatggggggggtaggt |
| | | cagagaagtcccactccagcatggctgcattgatcccccatcgttcccactagtctccgtaaaacctccca |
| | | gatacaggcacagtctagatgaaatcaggggtgcggggtgcaactgcaggccccaggcaattcaatagggg |
| | | ctctactttcacccccaggtcaccccagaatgctcacacaccagacactgacgccctggggctgtcaagat |
| | | caggcgtttgtctctgggcccagctcagggcccagctcagcacccactcagctcccctgaggctggggagc |
| | | ctgtcccattgcgactggagaggagagcggggccacagaggcctggctagaaggtcccttctccctggtgt |
| | | gtgttttctctctgctgagcaggcttgcagtgcctggggtatca |

TABLE 2
Amino Acid Sequence of Therapeutic Fusion Protein

| | | |
|---|---|---|
| 36 | therapeutic protein | MPSSVSWGILLLAGLCCLVPVSLAEDPQGDAAQKTDTSHHDQ |
| | | DHPTFNKITPNLAEFAFSLYRQLAHQSNSTNIFFSPVSIATAFAM |
| | | LSLGTKADTHDEILEGLNFNLTEIPEAQIHEGFQELLRTLNQPDS |
| | | QLQLTTGNGLFLSEGLKLVDKFLEDVKKLYHSEAFTVNFGDTE |
| | | EAKKQINDYVEKGTQGKIVDLVKELDRDTVFALVNYIFFKGK |
| | | WERPFEVKDTEEEDFHVDQVTTVKVPMMKRLGMFNIQHCKK |
| | | LSSWVLLMKYLGNATAIFFLPDEGKLQHLENELTHDIITKFLEN |
| | | EDRRSASLHLPKLSITGTYDLKSVLGQLGITKVFSNGADLSGVT |
| | | EEAPLKLSKAVHKAVLTIDEKGTEAAGAMFLEAIPMSIPPEVKF |
| | | NKPFVFLMIEQNTKSPLFMGKVVNPTQK |
| 37 | therapeutic | MYGKIIFVLLLSEIVSISAEDPQGDAAQKTDTSHHDQDHPTFNK |
| | protein-non- | ITPNLAEFAFSLYRQLAHQSNSTNIFFSPVSIATAFAMLSLGTKA |
| | cleavable linker- | DTHDEILEGLNFNLTEIPEAQIHEGFQELLRTLNQPDSQLQLTT |
| | GPA | GNGLFLSEGLKLVDKFLEDVKKLYHSEAFTVNFGDTEEAKKQI |
| | | NDYVEKGTQGKIVDLVKELDRDTVFALVNYIFFKGKWERPFE |
| | | VKDTEEEDFHVDQVTTVKVPMMKRLGMFNIQHCKKLSSWVL |
| | | LMKYLGNATAIFFLPDEGKLQHLENELTHDIITKFLENEDRRSA |
| | | SLHLPKLSITGTYDLKSVLGQLGITKVFSNGADLSGVTEEAPLK |
| | | LSKAVHKAVLTIDEKGTEAAGAMFLEAIPMSIPPEVKFNKPFVF |
| | | LMIEQNTKSPLFMGKVVNPTQKGSGGSGGSGLIIFGVMAGVIG |
| | | TILLISYGIRRL |
| 38 | therapeutic | MYGKIIFVLLLSEIVSISAEDPQGDAAQKTDTSHHDQDHPTFNK |
| | protein- | ITPNLAEFAFSLYRQLAHQSNSTNIFFSPVSIATAFAMLSLGTKA |
| | noncleavable | DTHDEILEGLNFNLTEIPEAQIHEGFQELLRTLNQPDSQLQLTT |
| | linker-GPA- | GNGLFLSEGLKLVDKFLEDVKKLYHSEAFTVNFGDTEEAKKQI |
| | GPA(C-term) | NDYVEKGTQGKIVDLVKELDRDTVFALVNYIFFKGKWERPFE |
| | | VKDTEEEDFHVDQVTTVKVPMMKRLGMFNIQHCKKLSSWVL |
| | | LMKYLGNATAIFFLPDEGKLQHLENELTHDIITKFLENEDRRSA |
| | | SLHLPKLSITGTYDLKSVLGQLGITKVFSNGADLSGVTEEAPLK |
| | | LSKAVHKAVLTIDEKGTEAAGAMFLEAIPMSIPPEVKFNKPFVF |
| | | LMIEQNTKSPLFMGKVVNPTQKGSGGSGGSGLIIFGVMAGVIG |
| | | TILLISYGIRRLIKKSPSDVKPLPSPDTDVPLSSVEIENPETSDQ |
| 39 | therapeutic | MYGKIIFVLLLSEIVSISAEDPQGDAAQKTDTSHHDQDHPTFNK |
| | protein-cleavable | ITPNLAEFAFSLYRQLAHQSNSTNIFFSPVSIATAFAMLSLGTKA |
| | linker-GPA | DTHDEILEGLNFNLTEIPEAQIHEGFQELLRTLNQPDSQLQLTT |
| | | GNGLFLSEGLKLVDKFLEDVKKLYHSEAFTVNFGDTEEAKKQI |
| | | NDYVEKGTQGKIVDLVKELDRDTVFALVNYIFFKGKWERPFE |
| | | VKDTEEEDFHVDQVTTVKVPMMKRLGMFNIQHCKKLSSWVL |
| | | LMKYLGNATAIFFLPDEGKLQHLENELTHDIITKFLENEDRRSA |
| | | SLHLPKLSITGTYDLKSVLGQLGITKVFSNGADLSGVTEEAPLK |
| | | LSKAVHKAVLTIDEKGTEAAGAMFLEAIPMSIPPEVKFNKPFVF |
| | | LMIEQNTKSPLFMGKVVNPTQKGSGGSGGSGPLGMWSRLIIFG |
| | | VMAGVIGTILLISYGIRRL |
| 40 | therapeutic | MYGKIIFVLLLSEIVSISAEDPQGDAAQKTDTSHHDQDHPTFNK |
| | protein-cleavable | ITPNLAEFAFSLYRQLAHQSNSTNIFFSPVSIATAFAMLSLGTKA |
| | linker-GPA- | DTHDEILEGLNFNLTEIPEAQIHEGFQELLRTLNQPDSQLQLTT |
| | GPA(C-term) | GNGLFLSEGLKLVDKFLEDVKKLYHSEAFTVNFGDTEEAKKQI |
| | | NDYVEKGTQGKIVDLVKELDRDTVFALVNYIFFKGKWERPFE |
| | | VKDTEEEDFHVDQVTTVKVPMMKRLGMFNIQHCKKLSSWVL |
| | | LMKYLGNATAIFFLPDEGKLQHLENELTHDIITKFLENEDRRSA |
| | | SLHLPKLSITGTYDLKSVLGQLGITKVFSNGADLSGVTEEAPLK |
| | | LSKAVHKAVLTIDEKGTEAAGAMFLEAIPMSIPPEVKFNKPFVF |
| | | LMIEQNTKSPLFMGKVVNPTQKGSGGSGGSGPLGMWSRLIIFG |
| | | VMAGVIGTILLISYGIRRLIKKSPSDVKPLPSPDTDVPLSSVEIEN |
| | | PETSDQ |
| 41 | 2A-therapeutic | EGRGSLLTCGDVEENPGPMPSSVSWGILLLAGLCCLVPVSLAE |
| | protein | DPQGDAAQKTDTSHHDQDHPTFNKITPNLAEFAFSLYRQLAH |
| | | QSNSTNIFFSPVSIATAFAMLSLGTKADTHDEILEGLNFNLTEIP |
| | | EAQIHEGFQELLRTLNQPDSQLQLTTGNGLFLSEGLKLVDKFL |
| | | EDVKKLYHSEAFTVNFGDTEEAKKQINDYVEKGTQGKIVDLV |
| | | KELDRDTVFALVNYIFFKGKWERPFEVKDTEEEDFHVDQVTT |
| | | VKVPMMKRLGMFNIQHCKKLSSWVLLMKYLGNATAIFFLPDE |
| | | GKLQHLENELTHDIITKFLENEDRRSASLHLPKLSITGTYDLKS |
| | | VLGQLGITKVFSNGADLSGVTEEAPLKLSKAVHKAVLTIDEKG |
| | | TEAAGAMFLEAIPMSIPPEVKFNKPFVFLMIEQNTKSPLFMGKV |
| | | VNPTQK |
| 42 | 2A-therapeutic | EGRGSLLTCGDVEENPGPMYGKIIFVLLLSEIVSISAEDPQGDA |
| | protein- | AQKTDTSHHDQDHPTFNKITPNLAEFAFSLYRQLAHQSNSTNIF |
| | noncleavable | FSPVSIATAFAMLSLGTKADTHDEILEGLNFNLTEIPEAQIHEGF |
| | linker-GPA | QELLRTLNQPDSQLQLTTGNGLFLSEGLKLVDKFLEDVKKLYH |
| | | SEAFTVNFGDTEEAKKQINDYVEKGTQGKIVDLVKELDRDTV |
| | | FALVNYIFFKGKWERPFEVKDTEEEDFHVDQVTTVKVPMMKR |
| | | LGMFNIQHCKKLSSWVLLMKYLGNATAIFFLPDEGKLQHLEN |
| | | ELTHDIITKFLENEDRRSASLHLPKLSITGTYDLKSVLGQLGITK |
| | | VFSNGADLSGVTEEAPLKLSKAVHKAVLTIDEKGTEAAGAMF |
| | | LEAIPMSIPPEVKFNKPFVFLMIEQNTKSPLFMGKVVNPTQKGS |
| | | GGSGGSGLIIFGVMAGVIGTILLISYGIRRL |
| 43 | 2A- -therapeutic | EGRGSLLTCGDVEENPGPMYGKIIFVLLLSEIVSISAEDPQGDA |
| | protein- | AQKTDTSHHDQDHPTFNKITPNLAEFAFSLYRQLAHQSNSTNIF |
| | noncleavable | FSPVSIATAFAMLSLGTKADTHDEILEGLNFNLTEIPEAQIHEGF |
| | linker-GPA- | QELLRTLNQPDSQLQLTTGNGLFLSEGLKLVDKFLEDVKKLYH |
| | GPA(C-term) | SEAFTVNFGDTEEAKKQINDYVEKGTQGKIVDLVKELDRDTV |
| | | FALVNYIFFKGKWERPFEVKDTEEEDFHVDQVTTVKVPMMKR |
| | | LGMFNIQHCKKLSSWVLLMKYLGNATAIFFLPDEGKLQHLEN |
| | | ELTHDIITKFLENEDRRSASLHLPKLSITGTYDLKSVLGQLGITK |
| | | VFSNGADLSGVTEEAPLKLSKAVHKAVLTIDEKGTEAAGAMF |
| | | LEAIPMSIPPEVKFNKPFVFLMIEQNTKSPLFMGKVVNPTQKGS |
| | | GGSGGSGLIIFGVMAGVIGTILLISYGIRRLIKKSPSDVKPLPSPD |
| | | TDVPLSSVEIENPETSDQ |
| 44 | 2A-therapeutic | EGRGSLLTCGDVEENPGPMYGKIIFVLLLSEIVSISAEDPQGDA |
| | protein-cleavable | AQKTDTSHHDQDHPTFNKITPNLAEFAFSLYRQLAHQSNSTNIF |
| | linker-GPA | FSPVSIATAFAMLSLGTKADTHDEILEGLNFNLTEIPEAQIHEGF |
| | | QELLRTLNQPDSQLQLTTGNGLFLSEGLKLVDKFLEDVKKLYH |
| | | SEAFTVNFGDTEEAKKQINDYVEKGTQGKIVDLVKELDRDTV |
| | | FALVNYIFFKGKWERPFEVKDTEEEDFHVDQVTTVKVPMMKR |
| | | LGMFNIQHCKKLSSWVLLMKYLGNATAIFFLPDEGKLQHLEN |
| | | ELTHDIITKFLENEDRRSASLHLPKLSITGTYDLKSVLGQLGITK |
| | | VFSNGADLSGVTEEAPLKLSKAVHKAVLTIDEKGTEAAGAMF |
| | | LEAIPMSIPPEVKFNKPFVFLMIEQNTKSPLFMGKVVNPTQKGS |
| | | GGSGGSGPLGMWSRLIIFGVMAGVIGTILLISYGIRRL |
| 45 | 2A-therapeutic | EGRGSLLTCGDVEENPGPMYGKIIFVLLLSEIVSISAEDPQGDA |
| | protein-cleavable | AQKTDTSHHDQDHPTFNKITPNLAEFAFSLYRQLAHQSNSTNIF |
| | linker-GPA- | FSPVSIATAFAMLSLGTKADTHDEILEGLNFNLTEIPEAQIHEGF |
| | GPA(C-term) | QELLRTLNQPDSQLQLTTGNGLFLSEGLKLVDKFLEDVKKLYH |
| | | SEAFTVNFGDTEEAKKQINDYVEKGTQGKIVDLVKELDRDTV |
| | | FALVNYIFFKGKWERPFEVKDTEEEDFHVDQVTTVKVPMMKR |
| | | LGMFNIQHCKKLSSWVLLMKYLGNATAIFFLPDEGKLQHLEN |
| | | ELTHDIITKFLENEDRRSASLHLPKLSITGTYDLKSVLGQLGITK |
| | | VFSNGADLSGVTEEAPLKLSKAVHKAVLTIDEKGTEAAGAMF |
| | | LEAIPMSIPPEVKFNKPFVFLMIEQNTKSPLFMGKVVNPTQKGS |
| | | GGSGGSGPLGMWSRLIIFGVMAGVIGTILLISYGIRRLIKKSPSD |
| | | VKPLPSPDTDVPLSSVEIENPETSDQ |
| 46 | Furin-2A- | RAKRGSGEGRGSLLTCGDVEENPGPMPSSVSWGILLLAGLCCL |
| | therapeutic protein | VPVSLAEDPQGDAAQKTDTSHHDQDHPTFNKITPNLAEFAFSL |
| | | YRQLAHQSNSTNIFFSPVSIATAFAMLSLGTKADTHDEILEGLN |
| | | FNLTEIPEAQIHEGFQELLRTLNQPDSQLQLTTGNGLFLSEGLK |
| | | LVDKFLEDVKKLYHSEAFTVNFGDTEEAKKQINDYVEKGTQG |
| | | KIVDLVKELDRDTVFALVNYIFFKGKWERPFEVKDTEEEDFHV |
| | | DQVTTVKVPMMKRLGMFNIQHCKKLSSWVLLMKYLGNATAI |
| | | FFLPDEGKLQHLENELTHDIITKFLENEDRRSASLHLPKLSITGT |
| | | YDLKSVLGQLGITKVFSNGADLSGVTEEAPLKLSKAVHKAVL |
| | | TIDEKGTEAAGAMFLEAIPMSIPPEVKFNKPFVFLMIEQNTKSP |
| | | LFMGKVVNPTQK |
| 47 | Furin- 2A- | RAKRGSGEGRGSLLTCGDVEENPGPMYGKIIFVLLLSEIVSISA |
| | therapeutic | EDPQGDAAQKTDTSHHDQDHPTFNKITPNLAEFAFSLYRQLAH |
| | protein- | QSNSTNIFFSPVSIATAFAMLSLGTKADTHDEILEGLNFNLTEIP |
| | noncleavable | EAQIHEGFQELLRTLNQPDSQLQLTTGNGLFLSEGLKLVDKFL |
| | linker-GPA | EDVKKLYHSEAFTVNFGDTEEAKKQINDYVEKGTQGKIVDLV |
| | | KELDRDTVFALVNYIFFKGKWERPFEVKDTEEEDFHVDQVTT |
| | | VKVPMMKRLGMFNIQHCKKLSSWVLLMKYLGNATAIFFLPDE |
| | | GKLQHLENELTHDIITKFLENEDRRSASLHLPKLSITGTYDLKS |
| | | VLGQLGITKVFSNGADLSGVTEEAPLKLSKAVHKAVLTIDEKG |
| | | TEAAGAMFLEAIPMSIPPEVKFNKPFVFLMIEQNTKSPLFMGKV |
| | | VNPTQKGSGGSGGSGLIIFGVMAGVIGTILLISYGIRRL |
| 48 | Furin-2A- - | RAKRGSGEGRGSLLTCGDVEENPGPMYGKIIFVLLLSEIVSISA |
| | therapeutic | EDPQGDAAQKTDTSHHDQDHPTFNKITPNLAEFAFSLYRQLAH |
| | protein- | QSNSTNIFFSPVSIATAFAMLSLGTKADTHDEILEGLNFNLTEIP |
| | noncleavable | EAQIHEGFQELLRTLNQPDSQLQLTTGNGLFLSEGLKLVDKFL |
| | linker-GPA- | EDVKKLYHSEAFTVNFGDTEEAKKQINDYVEKGTQGKIVDLV |
| | GPA(C-term) | KELDRDTVFALVNYIFFKGKWERPFEVKDTEEEDFHVDQVTT |
| | | VKVPMMKRLGMFNIQHCKKLSSWVLLMKYLGNATAIFFLPDE |
| | | GKLQHLENELTHDIITKFLENEDRRSASLHLPKLSITGTYDLKS |
| | | VLGQLGITKVFSNGADLSGVTEEAPLKLSKAVHKAVLTIDEKG |
| | | TEAAGAMFLEAIPMSIPPEVKFNKPFVFLMIEQNTKSPLFMGKV |
| | | VNPTQKGSGGSGGSGLIIFGVMAGVIGTILLISYGIRRLIKKSPS |
| | | DVKPLPSPDTDVPLSSVEIENPETSDQ |
| 49 | Furin-2A- | RAKRGSGEGRGSLLTCGDVEENPGPMYGKIIFVLLLSEIVSISA |
| | therapeutic | EDPQGDAAQKTDTSHHDQDHPTFNKITPNLAEFAFSLYRQLAH |
| | protein-cleavable | QSNSTNIFFSPVSIATAFAMLSLGTKADTHDEILEGLNFNLTEIP |
| | linker-GPA | EAQIHEGFQELLRTLNQPDSQLQLTTGNGLFLSEGLKLVDKFL |
| | | EDVKKLYHSEAFTVNFGDTEEAKKQINDYVEKGTQGKIVDLV |
| | | KELDRDTVFALVNYIFFKGKWERPFEVKDTEEEDFHVDQVTT |
| | | VKVPMMKRLGMFNIQHCKKLSSWVLLMKYLGNATAIFFLPDE |
| | | GKLQHLENELTHDIITKFLENEDRRSASLHLPKLSITGTYDLKS |
| | | VLGQLGITKVFSNGADLSGVTEEAPLKLSKAVHKAVLTIDEKG |
| | | TEAAGAMFLEAIPMSIPPEVKFNKPFVFLMIEQNTKSPLFMGKV |
| | | VNPTQKGSGGSGGSGPLGMWSRLIIFGVMAGVIGTILLISYGIR |
| | | RL |
| 69 | Furin- 2A- | RAKRGSGEGRGSLLTCGDVEENPGPMYGKIIFVLLLSEIVSISA |
| | therapeutic | EDPQGDAAQKTDTSHHDQDHPTFNKITPNLAEFAFSLYRQLAH |
| | protein-cleavable | QSNSTNIFFSPVSIATAFAMLSLGTKADTHDEILEGLNFNLTEIP |
| | linker-GPA- | EAQIHEGFQELLRTLNQPDSQLQLTTGNGLFLSEGLKLVDKFL |
| | GPA(C-term) | EDVKKLYHSEAFTVNFGDTEEAKKQINDYVEKGTQGKIVDLV |
| | | KELDRDTVFALVNYIFFKGKWERPFEVKDTEEEDFHVDQVTT |
| | | VKVPMMKRLGMFNIQHCKKLSSWVLLMKYLGNATAIFFLPDE |
| | | GKLQHLENELTHDIITKFLENEDRRSASLHLPKLSITGTYDLKS |
| | | VLGQLGITKVFSNGADLSGVTEEAPLKLSKAVHKAVLTIDEKG |
| | | TEAAGAMFLEAIPMSIPPEVKFNKPFVFLMIEQNTKSPLFMGKV |
| | | VNPTQKGSGGSGGSGPLGMWSRLIIFGVMAGVIGTILLISYGIR |
| | | RLIKKSPSDVKPLPSPDTDVPLSSVEIENPETSDQ |

TABLE 3
Additional Sequences

| | | | | Protein sequence |
|---|---|---|---|---|
| | SEQ ID NO | DNA sequence | SEQ ID NO | (if applicable) |
| Target sequence | 50 | ggcaagaagcatggc | | |
| for HBA 1 | | caccg | | |
| sgRNA | | | | |
| Target sequence | 51 | GCAGCATAGT | | |
| for CCR5 sgRNA | | GAGCCCAGAA | | |
| Homology arm | 52 | gctccagccggttcca | | |
| HBA1 5′ | | gctattgctttgtttacct | | |
| | | gtttaaccagtatttacc | | |
| | | tagcaagtcttccatca | | |
| | | gatagcatttggagag | | |
| | | ctgggggtgtcacagt | | |
| | | gaaccacgacctctag | | |
| | | gccagtgggagagtca | | |
| | | gtcacacaaactgtga | | |
| | | gtccatgacttggggct | | |
| | | tagccagcacccacca | | |
| | | ccccacgcgccaccc | | |
| | | cacaaccccgggtaga | | |
| | | ggagtctgaatctgga | | |
| | | gccgcccccagccca | | |
| | | gccccgtgctttttgcgt | | |
| | | cctggtgtttattccttcc | | |
| | | cggtgcctgtcactcaa | | |
| | | gcacactagtgactatc | | |
| | | gccagagggaaaggg | | |
| | | agctgcaggaagcga | | |
| | | ggctggagagcagga | | |
| | | ggggctctgcgcagaa | | |
| | | attcttttgagttcctatg | | |
| | | ggccagggcgtccgg | | |
| | | gtgcgcgcattcctctc | | |
| | | cgccccaggattgggc | | |
| | | gaagcctcccggctcg | | |
| | | cactcgctcgcccgtgt | | |
| | | gttccccgatcccgctg | | |
| | | gagtcgatgcgcgtcc | | |
| | | agcgcgtgccaggcc | | |
| | | ggggcgggggtgcgg | | |
| | | gctgactttctccctcgc | | |
| | | tagggacgctccggcg | | |
| | | cccgaaaggaaaggg | | |
| | | tggcgctgcgctccgg | | |
| | | ggtgcacgagccgac | | |
| | | agcgcccgaccccaa | | |
| | | cgggccggccccgcc | | |
| | | agcgccgctaccgccc | | |
| | | tgcccccgggcgagc | | |
| | | gggatggggggagt | | |
| | | ggagtggcgggtgga | | |
| | | gggtggagacgtcctg | | |
| | | gcccccgccccgcgt | | |
| | | gcacccccaggggag | | |
| | | gccgagcccgccgcc | | |
| | | cggccccgcgcaggc | | |
| | | cccgcccgggactccc | | |
| | | ctgcggtccaggccgc | | |
| | | gccccgggctccgcg | | |
| | | ccagccaatgagcgcc | | |
| | | gcccggccgggcgtg | | |
| | | cccccgcgccccaag | | |
| | | cataaaccctggcgcg | | |
| | | ctcgcggcccggcact | | |
| | | cttctggtccccacaga | | |
| | | ctcagagagaacccac | | |
| Homology arm | 53 | tggccatgcttcttgcc | | |
| HBA1 3′ | | ccttgggcctcccccc | | |
| | | agcccctcctccccttc | | |
| | | ctgcacccgtaccccc | | |
| | | gtggtctttgaataaagt | | |
| | | ctgagtgggcggcag | | |
| | | cctgtgtgtgcctgagtt | | |
| | | ttttccctcagcaaacgt | | |
| | | gccaggcatgggcgt | | |
| | | ggacagcagctggga | | |
| | | cacacatggctagaac | | |
| | | ctctctgcagctggata | | |
| | | gggtaggaaaaggca | | |
| | | ggggcgggaggagg | | |
| | | ggatggaggagggaa | | |
| | | agtggagccaccgcg | | |
| | | aagtccagctggaaaa | | |
| | | acgctggaccctagag | | |
| | | tgctttgaggatgcattt | | |
| | | gctctttcccgagttttat | | |
| | | tcccagacttttcagatt | | |
| | | caatgcaggtttgctga | | |
| | | aataatgaatttatccat | | |
| | | ctttacgtttctgggcac | | |
| | | tctgtgccaagaactgg | | |
| | | ctggctttctgcctggg | | |
| | | acgtcactggtttccca | | |
| | | gaggtcctcccacatat | | |
| | | gggtggtgggtaggtc | | |
| | | agagaagtcccactcc | | |
| | | agcatggctgcattgat | | |
| | | cccccatcgttcccact | | |
| | | agtctccgtaaaacctc | | |
| | | ccagatacaggcacag | | |
| | | tctagatgaaatcaggg | | |
| | | gtgcggggtgcaactg | | |
| | | caggccccaggcaatt | | |
| | | caataggggctctactt | | |
| | | tcacccccaggtcacc | | |
| | | ccagaatgctcacaca | | |
| | | ccagacactgacgccc | | |
| | | tggggctgtcaagatc | | |
| | | aggcgtttgtctctggg | | |
| | | cccagctcagggccca | | |
| | | gctcagcacccactca | | |
| | | gctcccctgaggctgg | | |
| | | ggagcctgtcccattgc | | |
| | | gactggagaggagag | | |
| | | cggggccacagaggc | | |
| | | ctggctagaaggtccct | | |
| | | tctccctggtgtgtgtttt | | |
| | | ctctctgctgagcaggc | | |
| | | ttgcagtgcctggggta | | |
| | | tca | | |
| Homology arm | 54 | GCTTTCATGA | | |
| CCR5 5′ | | ATTCCCCCAA | | |
| | | CAGAGCCAAG | | |
| | | CTCTCCATCT | | |
| | | AGTGGACAGG | | |
| | | GAAGCTAGCA | | |
| | | GCAAACCTTC | | |
| | | CCTTCACTAC | | |
| | | AAAACTTCAT | | |
| | | TGCTTGGCCA | | |
| | | AAAAGAGAGT | | |
| | | TAATTCAATG | | |
| | | TAGACATCTA | | |
| | | TGTAGGCAAT | | |
| | | TAAAAACCTA | | |
| | | TTGATGTATA | | |
| | | AAACAGTTTG | | |
| | | CATTCATGGA | | |
| | | GGGCAACTAA | | |
| | | ATACATTCTA | | |
| | | GGACTTTATA | | |
| | | AAAGATCACT | | |
| | | TTTTATTTATG | | |
| | | CACAGGGTGG | | |
| | | AACAAGATGG | | |
| | | ATTATCAAGT | | |
| | | GTCAAGTCCA | | |
| | | ATCTATGACA | | |
| | | TCAATTATTA | | |
| | | TACATCGGAG | | |
| | | CCCTGCCAAA | | |
| | | AAATCAATGT | | |
| | | GAAGCAAATC | | |
| | | GCAGCCCGCC | | |
| | | TCCTGCCTCC | | |
| | | GCTCTACTCA | | |
| | | CTGGTGTTCA | | |
| | | TCTTTGGTTTT | | |
| | | GTGGGCAACA | | |
| | | TGCTGGTCAT | | |
| | | CCTCATCCTG | | |
| | | ATAAACTGCA | | |
| | | AAAGGCTGAA | | |
| | | GAGCATGACT | | |
| | | GACATCTACC | | |
| | | TGCTCAACCT | | |
| | | GGCCATCTCT | | |
| | | GACCTGTTTTT | | |
| | | CCTTCTTACTG | | |
| | | TCCCCTTC | | |
| Homology arm | 55 | tgggctcactatgctgc | | |
| CCR5 3′ | | cgcccagtgggacttt | | |
| | | ggaaatacaatgtgtca | | |
| | | actcttgacagggctct | | |
| | | attttataggcttcttctct | | |
| | | ggaatcttcttcatcatc | | |
| | | ctcctgacaatcgatag | | |
| | | gtacctggctgtcgtcc | | |
| | | atgctgtgtttgctttaaa | | |
| | | agccaggacggtcac | | |
| | | ctttggggtggtgacaa | | |
| | | gtgtgatcacttgggtg | | |
| | | gtggctgtgtttgcgtct | | |
| | | ctcccaggaatcatcttt | | |
| | | accagatctcaaaaag | | |
| | | aaggtcttcattacacct | | |
| | | gcagctctcattttccat | | |
| | | acagtcagtatcaattct | | |
| | | ggaagaatttccagac | | |
| | | attaaagatagtcatctt | | |
| | | ggggctggtcctgccg | | |
| | | ctgcttgtcatggtcatc | | |
| | | tgctactcgggaatcct | | |
| | | aaaaactctgcttcggt | | |
| | | gtcgaaatgagaagaa | | |
| | | gaggcacagggctgt | | |
| | | gaggcttatcttcaccat | | |
| | | catgattgtttattttctct | | |
| | | tctgggctccctacaa | | |
| GPA | 56 | CTGATAATAT | 63 | LIIFGVMAGVI |
| transmembrane | | TCGGCGTTAT | | GTILLISYGIRR |
| domain | | GGCCGGCGTG | | L |
| | | ATCGGTACAA | | |
| | | TTCTGCTCATC | | |
| | | AGTTATGGGA | | |
| | | TACGGAGATT | | |
| | | G | | |
| GPA C-term | 57 | ATCAAGAAGA | 64 | IKKSPSDVKPL |
| | | GTCCTTCCGA | | PSPDTDVPLSS |
| | | CGTCAAGCCA | | VEIENPETSDQ |
| | | CTGCCAAGCC | | * |
| | | CAGATACCGA | | |
| | | TGTTCCGCTTT | | |
| | | CTTCAGTGGA | | |
| | | GATTGAGAAC | | |
| | | CCCGAAACTT | | |
| | | CTGACCAGTG | | |
| | | A | | |
| GPA signal | 58 | ATGTACGGGA | 65 | MYGKIIFVLLL |
| peptide | | AAATCATTTT | | SEIVSISA |
| | | CGTCCTGCTG | | |
| | | CTTTCTGAGA | | |
| | | TCGTTTCTATC | | |
| | | AGCGCT | | |
| GS linker | 59 | GGATCAGGCG | 66 | GSGGSGGSG |
| | | GATCCGGCGG | | |
| | | TAGCGGT | | |
| MMP cleavable | 60 | CCACTTGGTA | 67 | PLGMWSR |
| linker | | TGTGGTCTAG | | |
| | | G | | |
| Erythroid | 61 | GAACCTCAGA | | |
| specific promoter | | ACACTCAAAT | | |
| (GPA promoter) | | GATTTAAATT | | |
| | | TCTCAAATAC | | |
| | | ATTCATTTCA | | |
| | | CATATAGGAA | | |
| | | GTCACTTTCA | | |
| | | TTTGGACCAC | | |
| | | TGGGTCTTGA | | |
| | | CATTAGAAAT | | |
| | | GAGAAGGTCC | | |
| | | ATGGCTCCAC | | |
| | | AACAGCTACC | | |
| | | TCAGCCTGGC | | |
| | | ACGTGCCCTG | | |
| | | GCCTCAGAGA | | |
| | | TTCACAGTCC | | |
| | | AGTTCTTTGTC | | |
| | | CAGTTGGGTG | | |
| | | GCTCCTGTCT | | |
| | | ACCACCTTAC | | |
| | | CATGCCCACT | | |
| | | TAACTGATGC | | |
| | | AAAGTTAATA | | |
| | | TCACAAGTAG | | |
| | | CAACCTGTTC | | |
| | | CTTGCAGTGA | | |
| | | AAATTTTACT | | |
| | | TACCACTTTC | | |
| | | ATAGCCCCAA | | |
| | | GATATCCATG | | |
| | | TATCTTTATTA | | |
| | | ACAGGCGCTT | | |
| | | AACAACTTGC | | |
| | | ATCATTTAAA | | |
| | | ATGCCTCCCC | | |
| | | TGCCTATCAG | | |
| | | CTGATGATGG | | |
| | | CCGCAGGAAG | | |
| | | GTGGGCCTGG | | |
| | | AAGATAACAG | | |
| | | CTAGCAGGCT | | |
| | | AAGGTCAGAC | | |
| | | ACTGACACTT | | |
| | | GCAGTTGTCT | | |
| | | TTGGTAGTTTT | | |
| | | TTTGCACTAA | | |
| | | CTTCAGGAAC | | |
| | | CAGCTCATGA | | |
| | | TCTCAGG | | |
| AAT CDS | 62 | ATGCCTTCAT | 68 | MPSSVSWGILL |
| (including signal | | CAGTATCTTG | | LAGLCCLVPVS |
| peptide) | | GGGAATACTG | | LAEDPQGDAA |
| | | CTCCTTGCTG | | QKTDTSHHDQ |
| | | GGTTGTGTTG | | DHPTFNKITPN |
| | | TCTCGTACCC | | LAEFAFSLYRQ |
| | | GTGAGTCTCG | | LAHQSNSTNIF |
| | | CCGAAGACCC | | FSPVSIATAFA |
| | | TCAAGGCGAC | | MLSLGTKADT |
| | | GCCGCACAAA | | HDEILEGLNFN |
| | | AGACTGACAC | | LTEIPEAQIHEG |
| | | TTCTCATCAC | | FQELLRTLNQP |
| | | GACCAAGACC | | DSQLQLTTGN |
| | | ATCCTACATT | | GLFLSEGLKLV |
| | | TAATAAAATT | | DKFLEDVKKL |
| | | ACTCCAAATC | | YHSEAFTVNF |
| | | TCGCCGAATT | | GDTEEAKKQI |
| | | TGCGTTTTCTC | | NDYVEKGTQG |
| | | TGTATAGGCA | | KIVDLVKELDR |
| | | ACTCGCTCAC | | DTVFALVNYIF |
| | | CAATCTAATT | | FKGKWERPFE |
| | | CAACGAACAT | | VKDTEEEDFH |
| | | ATTCTTTTCAC | | VDQVTTVKVP |
| | | CTGTTTCCAT | | MMKRLGMFNI |
| | | AGCCACCGCT | | QHCKKLSSWV |
| | | TTCGCCATGC | | LLMKYLGNAT |
| | | TGAGTTTGGG | | AIFFLPDEGKL |
| | | AACAAAAGCA | | QHLENELTHDI |
| | | GATACCCATG | | ITKFLENEDRR |
| | | ACGAGATACT | | SASLHLPKLSIT |
| | | CGAAGGACTC | | GTYDLKSVLG |
| | | AACTTTAATC | | QLGITKVFSNG |
| | | TGACAGAAAT | | ADLSGVTEEAP |
| | | CCCTGAAGCA | | LKLSKAVHKA |
| | | CAAATTCACG | | VLTIDEKGTEA |
| | | AGGGTTTTCA | | AGAMFLEAIP |
| | | AGAGCTGCTG | | MISPPEVKFNK |
| | | AGAACTTTGA | | PFVFLMIEQNT |
| | | ATCAACCCGA | | KSPLFMGKVV |
| | | TTCCCAATTG | | NPTQK* |
| | | CAACTCACAA | | |
| | | CAGGAAACGG | | |
| | | TTTGTTTCTTT | | |
| | | CAGAAGGGCT | | |
| | | CAAACTGGTC | | |
| | | GACAAATTCC | | |
| | | TCGAAGACGT | | |
| | | GAAGAAACTT | | |
| | | TATCATAGCG | | |
| | | AGGCTTTTAC | | |
| | | CGTGAATTTT | | |
| | | GGAGATACGG | | |
| | | AAGAAGCTAA | | |
| | | GAAGCAAATA | | |
| | | AATGACTATG | | |
| | | TCGAAAAGGG | | |
| | | GACACAGGGA | | |
| | | AAGATAGTTG | | |
| | | ACCTGGTGAA | | |
| | | AGAACTGGAT | | |
| | | AGGGATACTG | | |
| | | TGTTCGCGCT | | |
| | | CGTCAACTAT | | |
| | | ATCTTCTTCA | | |
| | | AGGGGAAGTG | | |
| | | GGAACGGCCA | | |
| | | TTCGAGGTTA | | |
| | | AAGATACAGA | | |
| | | AGAGGAAGAT | | |
| | | TTTCATGTAG | | |
| | | ATCAAGTCAC | | |
| | | AACAGTCAAA | | |
| | | GTTCCAATGA | | |
| | | TGAAACGCCT | | |
| | | CGGGATGTTC | | |
| | | AATATACAAC | | |
| | | ATTGCAAGAA | | |
| | | ACTTAGCTCA | | |
| | | TGGGTCCTTTT | | |
| | | GATGAAGTAT | | |
| | | CTCGGGAACG | | |
| | | CTACAGCGAT | | |
| | | ATTCTTTCTCC | | |
| | | CAGACGAAGG | | |
| | | TAAGCTGCAA | | |
| | | CATCTTGAGA | | |
| | | ACGAGCTGAC | | |
| | | ACATGACATA | | |
| | | ATAACAAAAT | | |
| | | TTCTTGAGAA | | |
| | | CGAGGATCGC | | |
| | | CGGTCCGCAT | | |
| | | CCCTGCACCT | | |
| | | GCCGAAGCTT | | |
| | | AGCATAACCG | | |
| | | GCACATACGA | | |
| | | CTTGAAATCT | | |
| | | GTTCTTGGGC | | |
| | | AGCTTGGTAT | | |
| | | TACAAAAGTG | | |
| | | TTTTCCAACG | | |
| | | GCGCGGATCT | | |
| | | GTCAGGCGTG | | |
| | | ACGGAAGAAG | | |
| | | CTCCTCTTAA | | |
| | | ACTGAGTAAA | | |
| | | GCAGTCCACA | | |
| | | AAGCAGTACT | | |
| | | CACTATTGAT | | |
| | | GAAAAGGGTA | | |
| | | CCGAGGCGGC | | |
| | | CGGAGCTATG | | |
| | | TTCCTCGAAG | | |
| | | CTATTCCTAT | | |
| | | GAGTATTCCC | | |
| | | CCTGAAGTTA | | |
| | | AATTTAATAA | | |
| | | GCCTTTCGTG | | |
| | | TTTCTCATGAT | | |
| | | AGAGCAGAAC | | |
| | | ACGAAAAGCC | | |
| | | CTCTGTTTATG | | |
| | | GGCAAGGTCG | | |
| | | TCAACCCAAC | | |
| | | ACAGAAGTAA | | |

1. A method of expressing an exogenous protein of interest in a cell, the method comprising introducing into the cell:
i) a programmable nucleic acid-guided nuclease and an engineered guide polynucleotide, wherein the engineered guide polynucleotide hybridizes to a target sequence in an endogenous gene; and
ii) a donor polynucleotide sequence comprising:
a) an exogenous polynucleotide sequence encoding at least one therapeutic protein and a transmembrane domain, wherein the at least one therapeutic protein and the transmembrane domain are operably linked by a linker; and
b) 5′ homology and 3′ homology arms flanking the exogenous polynucleotide sequence, wherein the homology arms are homologous to portions of the endogenous gene;
whereupon generation of a double-strand break within the target sequence by the programmable nucleic acid-guided nuclease, the donor polynucleotide sequence is integrated into the endogenous gene locus by homology directed repair (HDR).

2. The method of claim 1, wherein the linker is a cleavable or a non-cleavable linker, wherein the non-cleavable linker is encoded by the nucleic acid sequence of SEQ ID NO: 59, which encodes the polypeptide sequence of SEQ ID NO: 66.

3. The method of claim 1, wherein the endogenous gene is the HBA1 gene or CCR5 gene.

4. (canceled)

5. The method of claim 1, wherein the programmable nuclease is a CRISPR-associated Cas protein.

6.-9. (canceled)

10. The method of claim 1, wherein the endogenous gene is a safe harbor site, wherein the safe harbor site is selected from the group consisting of: HBA1, HBA2, CCR5 locus, AAVS1, and the human ortholog of the murine Rosa26 locus.

11. (canceled)

12. The method of claim 1, wherein the engineered guide polynucleotide sequence is capable of hybridizing to a sequence having at least 95% sequence identity to SEQ ID NO: 50 or SEQ ID NO: 51.

13.-15. (canceled)

16. The method of claim 2, wherein the cleavable linker comprises at least one recognition motif for a protease, wherein the protease is selected from the group consisting of: metalloproteases, Serine proteases, Cysteine proteases, threonine proteases, Aspartic proteases, Glutamic proteases, and Asparagine proteases.

17. (canceled)

18. The method of claim 1, wherein the linker is a matrix metalloproteinase (MMP) linker.

19. The method of claim 1, wherein the therapeutic protein comprises alpha-antitrypsin (AAT) or an active variant or portion thereof, wherein the AAT is encoded by a polynucleotide sequence having at least 75% sequence identity to SEQ ID NO: 62.

20.-22. (canceled)

23. The method of claim 1, wherein the transmembrane domain comprises a glycophorin A (GPA) transmembrane domain, wherein nucleic acid sequence encoding the GPA transmembrane domain has at least 75% sequence identity to SEQ ID NO: 56 or SEQ ID NO: 63.

24.-25. (canceled)

26. The method of claim 1, wherein the exogenous polynucleotide sequence further comprises a nucleic acid sequence encoding a C-terminal tail, wherein the C-terminal tail is encoded by a polynucleotide sequence having at least 75% sequence identity SEQ ID NO: 57 or SEQ ID NO: 64.

27.-28. (canceled)

29. The method of claim 1, wherein:
the 5′ homology arm comprises a polynucleotide sequence having at least 75% sequence identity to SEQ ID NO: 52 or SEQ ID NO: 54;
the 3′ homology arm comprises a polynucleotide sequence having at least 75% sequence identity to SEQ ID NO: 53 or SEQ ID NO: 55; or
any combination thereof.

30.-32. (canceled)

33. The method of a claim 1, wherein the donor polynucleotide comprises, in a 5′ to 3′ orientation:
a) a 5′ homology arm, promoter, therapeutic protein, cleavable linker, GPA transmembrane domain, GPA C-terminal tail, and a 3′ homology arm;
b) a 5′ homology arm, promoter, therapeutic protein, non-cleavable linker, GPA transmembrane domain, GPA C-terminal tail, and a 3′ homology arm;
c) a 5′ homology arm, promoter, therapeutic protein, cleavable linker, GPA transmembrane domain, and a 3′ homology arm;
d) a 5′ homology arm, promoter, therapeutic protein, non-cleavable linker, and a GPA-3′ homology arm;
e) a 5′ homology arm, therapeutic protein, cleavable linker, GPA transmembrane domain, GPA C-terminal tail, and a 3′ homology arm;
f) a 5′ homology arm, therapeutic protein, non-cleavable linker, GPA transmembrane domain, GPA C-terminal tail, and a 3′ homology arm;
g) a 5′ homology arm, therapeutic protein, cleavable linker, GPA transmembrane domain, and a 3′ homology arm; or
h) a 5′ homology arm, therapeutic protein, non-cleavable linker, GPA transmembrane domain, and a 3′ homology arm.

34.-37. (canceled)

38. A genetically modified HSPC, prepared according to the method of claim 1, wherein the HSPC expresses a polypeptide comprising a transmembrane domain and a therapeutic protein, wherein the transmembrane domain and therapeutic protein are operably linked by a linker.

39. (canceled)

40. The genetically modified HSPC of claim 38, wherein the genetically modified HSPC can be further differentiated into an erythrocyte.

41. (canceled)

42. An exogenous protein cell expression kit, comprising the method of claim 1.

43. A donor polynucleotide sequence comprising:
a. an exogenous polynucleotide sequence encoding at least one therapeutic protein and a transmembrane domain, wherein the at least one therapeutic protein and the transmembrane domain are operably linked by a linker; and
b. 5′ and 3′ homology arms flanking the exogenous polynucleotide sequence, wherein the homology arms are homologous to a portion of an endogenous gene.

44.-58. (canceled)

59. The donor polynucleotide of claim 43, wherein the 5′ and 3′ homology arms are homologous to portions of the HBA1 gene or CCR5 gene.

60.-64. (canceled)

65. The donor polynucleotide of claim 43, wherein the donor polynucleotide sequence comprises a polynucleotide sequence which encodes a polypeptide sequence having at least 75% sequence identity to any one of SEQ ID NOs: 1-49, or 69.

66. A method of treating alpha-antitrypsin deficiency in a subject in need thereof, the method comprising:
i) introducing into an HSPC a nucleic acid-guide programmable nuclease and an engineered guide polynucleotide capable of hybridizing to a target sequence of an endogenous gene selected from the group consisting of SEQ ID NO: 50 or SEQ ID NO: 51; and
ii) introducing a recombinant AAV6 vector comprising a donor polynucleotide sequence into the HSPC, wherein the donor polynucleotide comprises an exogenous polynucleotide sequence comprising a sequence selected from the group consisting of: NO 1 to SEQ ID NO: 35,
whereupon generation of a double-strand break within the target sequence by the programmable nucleic acid-guided nuclease, the donor polynucleotide sequence is integrated into the endogenous gene locus by homology directed repair (HDR), thereby generating a genetically modified HSPC; and
iii) introducing the genetically modified HSPC into the subject.

67. (canceled)
